SEQUENCE LISTING (96 sequences)

SEQ ID NO: 1 - RNA, 20 nt, synthetic construct
  source 1..20: mol_type = other RNA
agaccggttctggagacaaa

SEQ ID NO: 2 - RNA, 20 nt, synthetic construct
  source 1..20: mol_type = other RNA
gctctgggactcacattgct

SEQ ID NO: 3 - RNA, 20 nt, synthetic construct
  source 1..20: mol_type = other RNA
tcccagaatcccttgtccct

SEQ ID NO: 4 - RNA, 19 nt, synthetic construct
  source 1..19: mol_type = other RNA
aggcttcctcctcctcacc

SEQ ID NO: 5 - RNA, 20 nt, synthetic construct
  source 1..20: mol_type = other RNA
cgcggtcgccaaaaagaaag

SEQ ID NO: 6 - RNA, 846 nt, Homo sapiens
  source 1..846: mol_type = mRNA
agaccggttctggagacaaaaggggccgcggcggccggagcgggacgggcccggcgcgggagggagcgaagcagcgcgggcagcgagcgagatgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttccccccaaaaaaaaaaaaaaaaaa

SEQ ID NO: 7 - DNA, 56 bp, Homo sapiens
  source 1..56: mol_type = genomic DNA
gcagggtgccctgcaactggaagaaggagtttggaggtgaggcggggcgcagtcag

SEQ ID NO: 8 - DNA, 35 bp, Homo sapiens
  source 1..35: mol_type = genomic DNA
gcagggtgccctgcaactggaagaaggagtttgga

SEQ ID NO: 9 - DNA, 30 bp, Homo sapiens
  source 1..30: mol_type = genomic DNA
gcagggtgccctgcaactggaagaaggagt

SEQ ID NO: 10 - DNA, 22 bp, Homo sapiens
  source 1..22: mol_type = genomic DNA
caactggaagaaggagtttgga

SEQ ID NO: 11 - DNA, 33 bp, Homo sapiens
  source 1..33: mol_type = genomic DNA
caactggaagaaggagtttggaggtgaggcggg

SEQ ID NO: 12 - DNA, 41 bp, Homo sapiens
  source 1..41: mol_type = genomic DNA
actggaagaaggagtttggaggtgaggcggggcgcagtcag

SEQ ID NO: 13 - DNA, 31 bp, Homo sapiens
  source 1..31: mol_type = genomic DNA
actggaagaaggagtttggaggtgaggcggg

SEQ ID NO: 14 - DNA, 30 bp, Homo sapiens
  source 1..30: mol_type = genomic DNA
gagtttggaggtgaggcggggcgcagtcag

SEQ ID NO: 15 - DNA, 114 bp, Homo sapiens
  source 1..114: mol_type = genomic DNA
ggcaccctgaagaaggcgcgctacaatgctcagtgccaggagaccatccgcgtcaccaagccctgcacccccaagaccaaagcaaaggccaaaggtcagcgaaaggagaagggg

SEQ ID NO: 16 - DNA, 67 bp, Homo sapiens
  source 1..67: mol_type = genomic DNA
ggcaccctgaagaaggcgcgctacaatgctcagtgccaggagaccatccgcgtcaccaagccctgca

SEQ ID NO: 17 - DNA, 35 bp, Homo sapiens
  source 1..35: mol_type = genomic DNA
ggcaccctgaagaaggcgcgctacaatgctcagtg

SEQ ID NO: 18 - DNA, 35 bp, Homo sapiens
  source 1..35: mol_type = genomic DNA
gtgccaggagaccatccgcgtcaccaagccctgca

SEQ ID NO: 19 - DNA, 35 bp, Homo sapiens
  source 1..35: mol_type = genomic DNA
aagcaaaggccaaaggtcagcgaaaggagaagggg

SEQ ID NO: 20 - DNA, 25 bp, Homo sapiens
  source 1..25: mol_type = genomic DNA
cgcgcgttgtagataaggtgaagaa

SEQ ID NO: 21 - DNA, 25 bp, Homo sapiens
  source 1..25: mol_type = genomic DNA
gtagataaggtgaagaagggcggcc

SEQ ID NO: 22 - DNA, 25 bp, Homo sapiens
  source 1..25: mol_type = genomic DNA
tttccgcgagggcacctgcggggcc

SEQ ID NO: 23 - DNA, 25 bp, Homo sapiens
  source 1..25: mol_type = genomic DNA
ctgcaactggaagaaggagtttgga

SEQ ID NO: 24 - DNA, 20 bp, Homo sapiens
  source 1..20: mol_type = genomic DNA
caactggaagaaggagtttg

SEQ ID NO: 25 - DNA, 25 bp, Homo sapiens
  source 1..25: mol_type = genomic DNA
agaaggagtttggaggtgaggcggg

SEQ ID NO: 26 - DNA, 25 bp, Homo sapiens
  source 1..25: mol_type = genomic DNA
ctctctcgccagccgactgcaagta

SEQ ID NO: 27 - DNA, 25 bp, Homo sapiens
  source 1..25: mol_type = genomic DNA
ccagccgactgcaagtacaagtttg

SEQ ID NO: 28 - DNA, 25 bp, Homo sapiens
  source 1..25: mol_type = genomic DNA
cctgaagaaggcgcgctacaatgct

SEQ ID NO: 29 - DNA, 25 bp, Homo sapiens
  source 1..25: mol_type = genomic DNA
aggagaccatccgcgtcaccaagcc

SEQ ID NO: 30 - DNA, 25 bp, Homo sapiens
  source 1..25: mol_type = genomic DNA
aaggccaaaggtcagcgaaaggaga

SEQ ID NO: 31 - DNA, 25 bp, Homo sapiens
  source 1..25: mol_type = genomic DNA
gcagggtgccctgcaactggaagaa

SEQ ID NO: 32 - DNA, 25 bp, Homo sapiens
  source 1..25: mol_type = genomic DNA
gtgccctgcaactggaagaaggagt

SEQ ID NO: 33 - DNA, 25 bp, Homo sapiens
  source 1..25: mol_type = genomic DNA
actggaagaaggagtttggaggtga

SEQ ID NO: 34 - DNA, 25 bp, Homo sapiens
  source 1..25: mol_type = genomic DNA
gagtttggaggtgaggcggggcgca

SEQ ID NO: 35 - DNA, 25 bp, Homo sapiens
  source 1..25: mol_type = genomic DNA
gagtttggaggtgaggcggggcgca

SEQ ID NO: 36 - DNA, 25 bp, Homo sapiens
  source 1..25: mol_type = genomic DNA
ggcaccctgaagaaggcgcgctaca

SEQ ID NO: 37 - DNA, 25 bp, Homo sapiens
  source 1..25: mol_type = genomic DNA
agaaggcgcgctacaatgctcagtg

SEQ ID NO: 38 - DNA, 25 bp, Homo sapiens
  source 1..25: mol_type = genomic DNA
gtgccaggagaccatccgcgtcacc

SEQ ID NO: 39 - DNA, 25 bp, Homo sapiens
  source 1..25: mol_type = genomic DNA
accatccgcgtcaccaagccctgca

SEQ ID NO: 40 - DNA, 25 bp, Homo sapiens
  source 1..25: mol_type = genomic DNA
aagcaaaggccaaaggtcagcgaaa

SEQ ID NO: 41 - DNA, 25 bp, Homo sapiens
  source 1..25: mol_type = genomic DNA
caaaggtcagcgaaaggagaagggg

SEQ ID NO: 42 - DNA, 25 bp, Homo sapiens
  source 1..25: mol_type = genomic DNA
agtgccaggagaccatccgcgtcac

SEQ ID NO: 43 - RNA, 25 nt, synthetic construct
  source 1..25: mol_type = other RNA
ttcttcaccttatctacaacgcgcg

SEQ ID NO: 44 - RNA, 25 nt, synthetic construct
  source 1..25: mol_type = other RNA
ggccgcccttcttcaccttatctac

SEQ ID NO: 45 - RNA, 25 nt, synthetic construct
  source 1..25: mol_type = other RNA
ggccccgcaggtgccctcgcggaaa

SEQ ID NO: 46 - RNA, 25 nt, synthetic construct
  source 1..25: mol_type = other RNA
tccaaactccttcttccagttgcag

SEQ ID NO: 47 - RNA, 20 nt, synthetic construct
  source 1..20: mol_type = other RNA
aaactccttcttccagttgc

SEQ ID NO: 48 - RNA, 25 nt, synthetic construct
  source 1..25: mol_type = other RNA
cccgcctcacctccaaactccttct

SEQ ID NO: 49 - RNA, 25 nt, synthetic construct
  source 1..25: mol_type = other RNA
tacttgcagtcggctggcgagagag

SEQ ID NO: 50 - RNA, 25 nt, synthetic construct
  source 1..25: mol_type = other RNA
caaacttgtacttgcagtcggctgg

SEQ ID NO: 51 - RNA, 25 nt, synthetic construct
  source 1..25: mol_type = other RNA
agcattgtagcgcgccttcttcagg

SEQ ID NO: 52 - RNA, 25 nt, synthetic construct
  source 1..25: mol_type = other RNA
ggcttggtgacgcggatggtctcct

SEQ ID NO: 53 - RNA, 25 nt, synthetic construct
  source 1..25: mol_type = other RNA
tctcctttcgctgacctttggcctt

SEQ ID NO: 54 - RNA, 25 nt, synthetic construct
  source 1..25: mol_type = other RNA
ttcttccagttgcagggcaccctgc

SEQ ID NO: 55 - RNA, 25 nt, synthetic construct
  source 1..25: mol_type = other RNA
actccttcttccagttgcagggcac

SEQ ID NO: 56 - RNA, 25 nt, synthetic construct
  source 1..25: mol_type = other RNA
tcacctccaaactccttcttccagt

SEQ ID NO: 57 - RNA, 25 nt, synthetic construct
  source 1..25: mol_type = other RNA
tgcgccccgcctcacctccaaactc

SEQ ID NO: 58 - RNA, 25 nt, synthetic construct
  source 1..25: mol_type = other RNA
ctgactgcgccccgcctcacctcca

SEQ ID NO: 59 - RNA, 25 nt, synthetic construct
  source 1..25: mol_type = other RNA
tgtagcgcgccttcttcagggtgcc

SEQ ID NO: 60 - RNA, 25 nt, synthetic construct
  source 1..25: mol_type = other RNA
cactgagcattgtagcgcgccttct

SEQ ID NO: 61 - RNA, 25 nt, synthetic construct
  source 1..25: mol_type = other RNA
ggtgacgcggatggtctcctggcac

SEQ ID NO: 62 - RNA, 25 nt, synthetic construct
  source 1..25: mol_type = other RNA
tgcagggcttggtgacgcggatggt

SEQ ID NO: 63 - RNA, 25 nt, synthetic construct
  source 1..25: mol_type = other RNA
tttcgctgacctttggcctttgctt

SEQ ID NO: 64 - RNA, 25 nt, synthetic construct
  source 1..25: mol_type = other RNA
ccccttctcctttcgctgacctttg

SEQ ID NO: 65 - RNA, 25 nt, synthetic construct
  source 1..25: mol_type = other RNA
gtgacgcggatggtctcctggcact

SEQ ID NO: 66 - RNA, 90 nt, Homo sapiens
  source 1..90: mol_type = mRNA
agaccggttctggagacaaaaggggccgcggcggccggagcgggacgggcccggcgcgggagggagcgaagcagcgcgggcagcgagcga

SEQ ID NO: 67 - RNA, 77 nt, Homo sapiens
  source 1..77: mol_type = mRNA
gatgcagcaccgaggcttcctcctcctcaccctcctcgccctgctggcgctcacctccgcggtcgccaaaaagaaag

SEQ ID NO: 68 - protein, 25 aa, Homo sapiens
MQHRGFLLLTLLALLALTSAVAKKK

SEQ ID NO: 69 - RNA, 168 nt, Homo sapiens
  source 1..168: mol_type = mRNA
ataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggag

SEQ ID NO: 70 - protein, 55 aa, Homo sapiens
KVKKGGPGSECAEWAWGPCTPSSKDCGVGFREGTCGAQTQRIRCRVPCNWKKEFG

SEQ ID NO: 71 - DNA, 162 bp, Homo sapiens
  source 1..162: mol_type = genomic DNA
ccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaag

SEQ ID NO: 72 - RNA, 162 nt, Homo sapiens
  source 1..162: mol_type = mRNA
ccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaag

SEQ ID NO: 73 - protein, 53 aa, Homo sapiens
DCKYKFENWGACDGGTGTKVRQGTLKKARYNAQCQETIRVTKPCTPKTKAKAK

SEQ ID NO: 74 - RNA, 378 nt, Homo sapiens
  source 1..378: mol_type = mRNA
accaagccctgcacccccaagaccaaagcaaaggccaaagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttccccccaaaaaaa

SEQ ID NO: 75 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 76 - DNA, 684 bp, Homo sapiens
  source 1..684: mol_type = genomic DNA
agaccggttctggagacaaaaggggccgcggcggccggagcgggacgggcccggcgcgggagggagcgaagcagcgcgggcagcgagcgagatgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttccccccaaaaaaaaaaaaaaaaaa

SEQ ID NO: 77 - RNA, 684 nt, Homo sapiens
  source 1..684: mol_type = mRNA
agaccggttctggagacaaaaggggccgcggcggccggagcgggacgggcccggcgcgggagggagcgaagcagcgcgggcagcgagcgagatgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttccccccaaaaaaaaaaaaaaaaaa

SEQ ID NO: 78 - protein, 89 aa, Homo sapiens
MQHRGFLLLTLLALLALTSAVAKKKDKVKKGGPGSECAEWAWGPCTPSSKDCGVGFREGTCGAQTQRIRCRVPCNWKKEFGAKKGKGKD

SEQ ID NO: 79 - DNA, 17 bp, Homo sapiens
  source 1..17: mol_type = genomic DNA
ccgactgcaagtacaag

SEQ ID NO: 80 - DNA, 16 bp, Homo sapiens
  source 1..16: mol_type = genomic DNA
aaagcaaaggccaaag

SEQ ID NO: 81 - RNA, 17 nt, Homo sapiens
  source 1..17: mol_type = mRNA
ccgactgcaagtacaag

SEQ ID NO: 82 - RNA, 16 nt, Homo sapiens
  source 1..16: mol_type = mRNA
aaagcaaaggccaaag

SEQ ID NO: 83 - protein, 5 aa, Homo sapiens
DCKYK

SEQ ID NO: 84 - protein, 5 aa, Homo sapiens
KAKAK

SEQ ID NO: 85 - protein, 8 aa, Homo sapiens
AKKGKGKD

SEQ ID NO: 86 - RNA, 390 nt, Homo sapiens
  source 1..390: mol_type = mRNA
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagtag

SEQ ID NO: 87 - protein, 129 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPK

SEQ ID NO: 88 - RNA, 357 nt, Homo sapiens
  source 1..357: mol_type = mRNA
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagtag

SEQ ID NO: 89 - protein, 118 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQE

SEQ ID NO: 90 - RNA, 330 nt, Homo sapiens
  source 1..330: mol_type = mRNA
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaagtag

SEQ ID NO: 91 - protein, 109 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKK

SEQ ID NO: 92 - RNA, 309 nt, Homo sapiens
  source 1..309: mol_type = mRNA
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtctag

SEQ ID NO: 93 - protein, 102 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKV

SEQ ID NO: 94 - RNA, 258 nt, Homo sapiens
  source 1..258: mol_type = mRNA
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtag

SEQ ID NO: 95 - protein, 85 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCK

SEQ ID NO: 96 - RNA, 432 nt, Homo sapiens
  source 1..432: mol_type = mRNA
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactag

(19) United States
(12) Patent Application Publication
(10) Pub. No.: US 2026/0049113 A1
(43) Pub. Date: Feb. 19, 2026

(54) COMPOSITIONS AND METHODS FOR DELIVERY OF TRUNCATED MIDKINE PROTEINS

(71) Applicant: LYRAMID PTY LTD, West Leederville (AU)

(72) Inventor: Graham ROBERTSON, West Leederville (AU)

(21) Appl. No.: 19/102,805

(22) PCT Filed: Aug. 8, 2023
(86) PCT No.: PCT/AU2023/050739

§ 371 Date: Feb. 10, 2025

(30) Foreign Application Priority Data
Aug. 9, 2022 (AU) 2022902231
Jun. 9, 2023 (AU) 2023901841

(51) Int. Cl. C07K 14/475; A61K 31/7105; A61K 35/00; A61K 38/00; C12N 15/88

(52) U.S. Cl. CPC C07K 14/475; A61K 31/7105; A61K 35/00; C12N 15/88; A61K 38/00; C12N 2800/22

(57) ABSTRACT

The present disclosure relates generally to novel compositions and methods for delivery of truncated midkine proteins in which the amino acid sequence encoded by exon 4 of human midkine is absent or partially absent and/or polynucleotides encoding said truncated human midkine proteins.

### RELATED APPLICATION DATA

The present application claims priority from Australian Patent Application No. 2022902231 entitled ‘Compositions and Methods for Delivery of Truncated Midkine Proteins’ filed 9 Aug. 2022 and Australian Patent Application No. 2023901841 entitled ‘Compositions and Methods for Delivery of Truncated Midkine Proteins’ filed 9 Jun. 2023. The entire contents of which is hereby incorporated by reference.

### SEQUENCE LISTING

The present application is filed with a Sequence Listing in electronic form. The entire contents of the Sequence Listing are hereby incorporated by reference.

### TECHNICAL FIELD

The present disclosure relates generally to novel compositions and methods for delivery of truncated midkine proteins. In particular, the present disclosure relates to compositions comprising truncated human midkine proteins in which the amino acid sequence encoded by exon 4 of human midkine is absent or partially absent and/or polynucleotides encoding said truncated human midkine proteins. The present disclosure also relates to the use of those compositions to treat individuals with a midkine-related disease or disorder or who are predisposed thereto.

### BACKGROUND

Midkine (MDK) is a heparin-binding growth factor found as a product of a gene transiently expressed in the stage of retinoic acid-induced differentiation of embryonal carcinoma (EC) cells and is a polypeptide of 13 kDa in molecular weight rich in basic amino acids and cysteine (Kadomatsu. et al. (1988) Biochem. Biophys. Res. Commun., 151:1312-1318; Tomokura et al. (1999) J. Biol. Chem, 265:10765-10770; Muramatsu T (2014) Brit J Pharmacol 171:814-826).

Midkine is known to have various biological activities. For example, it is known that midkine expression is increased in human cancer cells. This increase in expression has been confirmed in various cancers such as esophageal cancer, thyroid cancer, urinary bladder cancer, colorectal cancer, stomach cancer, pancreatic cancer, thoracic cancer, liver cancer, lung cancer, breast cancer, neuroblastoma, glioblastoma, mesothelioma, renal cancer, head and neck cancer, melanoma, uterine/cervical cancer, ovarian cancer, osteosarcoma, chronic lymphocytic leukaemia and Wilms tumour (Muramatsu (2002) J. Biochem. 132:359-371; Jones (2014) Brit J Pharm 171:2925-2939). Moreover, midkine enhances the proliferation, survival and migration of cancer cells; promotes angiogenesis; contributes to cancer progression and metastasis; as well as modulates the tumour immune microenvironment. Midkine is also a major determinant of response to cancer treatment, including chemotherapy and immunotherapy.

Midkine is also known to play a central role in regulating immune and inflammatory responses (Heradon G et al (2019) Frontier Pharmacol 10:377; Aynacioglu A S et al (2018) Modern Rheumatology 29:567-571; Sorrelle N et al (2017) J Leukoc Biol 102:277-286). For example, it is known that neointimal formation after vascular injury and nephritis onset during ischemic injury are suppressed in knockout mice deficient in midkine genes. Moreover, it is also known that rheumatism models and postoperative adhesions are significantly suppressed in such knockout mice (WO2000/10608; WO2004/078210). Thus, midkine is known to participate in inflammatory diseases and autoimmune disorders such as arthritis (both Rheumatoid and Osteoarthritis), postoperative adhesion, inflammatory bowel disease, autoimmune myocarditis, chronic kidney disease, psoriasis, lupus, asthma, and multiple sclerosis involving T regulatory cell dysfunction (Takeuchi H (2014) Brit J Pharmacol 171:931-935). Furthermore, midkine is known to promote the migration, activation and functional orientation of inflammatory cells such as macrophages or neutrophils. Since recruitment and deleterious behaviour of neutrophils and macrophages are necessary for the establishment of inflammatory responses in diseased tissues, deficiency or blockade of midkine action prevents diseases based on inflammation in animal models (WO1999/03493). However, there are no midkine-based therapies that have progressed beyond preclinical experimental testing. This limitation is exemplified by monoclonal antibodies targeting midkine that have only shown benefit when administered in prophylactic mode and failed in treatment mode once the disease or tumour is established.

Accordingly, there remains a need for new compositions and methods with improved ability to modulate, inhibit or reduce the abundance and activity of functional midkine. There is also a need for compositions having the ability to modulate, inhibit or reduce the abundance and activity of functional midkine that are cost effective and/or simple to manufacture.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure is based, inter alia, on a recognition by the inventors that there is a need for novel therapeutic strategies to target midkine (MDK) activity or function and treat conditions associated with midkine action. To this end, the inventors have developed novel compositions which are configured to deliver truncated forms of midkine protein, in particular truncated forms of midkine protein in which the amino acid sequence encoded by exon 4 of human midkine is absent, partially absent or substantially absent. Whilst not wishing to be bound to any one theory, it is believed that the truncated forms of midkine protein of the disclosure act as a dominant negative antagonist. The biologically active form of midkine is thought to be a homodimer comprising two full length monomers. In this regard, it is believed that the truncated forms of midkine protein of the disclosure are non-functional but retain the ability to form heterodimers with full-length functional midkine protein, thereby inhibiting the activity of the full length human midkine in a cell or tissue. Alternatively, the truncated midkine protein may interfere or compete with the ability of full length midkine to interact with a ligand thereof on the surface of, or in, a cell.

In an effort to produce truncated forms of midkine protein of the disclosure, the inventors have developed midkine mRNAs that translate into forms of midkine protein that lack critical functional domains, thereby reducing the biological activity of the full length midkine protein. The presence of the truncated forms of midkine protein was also confirmed in Western blots using a polyclonal antibody that recognizes epitopes in regions retained in the truncated midkine protein (e.g. N-terminal region). Uniquely, these midkine mRNAs and truncated midkine protein variants are distinguished from naturally occurring midkine splice variants as they leave the N-terminal domain intact, and only involve removal of sequences from the C-terminal domain of midkine. Furthermore, the midkine mRNAs and truncated midkine protein variants disclosed herein do not involve addition of any amino acid or polynucleotide sequences that are not related to midkine.

The inventors surprisingly found that specific truncated variants were able to inhibit midkine function better than other variants, and in particular inhibit midkine-mediated cell migration and/or reduce cancer cell survival.

Thus, the present disclosure provides a composition comprising:
(i) a truncated human midkine protein in which the amino acid sequence encoded by exon 4 of human midkine is absent, partially absent or substantially absent; and/or
(ii) a polynucleotide encoding the truncated human midkine protein at (i).

In one example, the composition comprises a truncated human midkine protein in which the amino acid sequence encoded by exon 4 of human midkine is absent, partially absent or substantially absent.

In one example, the amino acid sequence encoded by exon 4 of human midkine protein is substantially absent from the truncated human midkine protein. The amino acid sequence encoded by exon 4 of human midkine is set forth in SEQ ID NO: 73. In accordance with certain examples in which the amino acid sequence encoded by exon 4 of human midkine protein is substantially absent, at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% of the sequence set forth in SEQ ID NO: 73 is absent from the truncated human midkine protein. In another example, the amino acid sequence encoded by exon 4 of human midkine protein (i.e., the amino acid sequence set forth in SEQ ID NO: 73) is absent from the truncated human midkine protein.

In accordance with examples in which the amino acid sequence encoded by exon 4 is substantially absent, one or more of the amino acids within exon 4 and adjacent to the exon 3/exon 4 boundary may be present within the truncated midkine protein. For example, 1, 2, 3, 4 or 5 contiguous amino acids of the amino acid sequence set forth in SEQ ID NO: 83 may be present within the truncated human midkine protein. In some examples, the amino acid sequence set forth in SEQ ID NO: 83 is present within the truncated human midkine protein. Alternatively, or in addition, where the amino acid sequence encoded by exon 4 is substantially absent, one or more amino acids within exon 4 which are adjacent to the exon 4/exon 5 boundary may be present. For example, 1, 2, 3, 4 or 5 contiguous amino acids of the amino acid sequence set forth in SEQ ID NO: 84 may be present within the truncated human midkine protein. In yet another example, one or more amino acids within exon 4 and adjacent to the exon 3/exon 4 boundary may also be present within the truncated midkine protein, and one or more amino acids within exon 4 which are adjacent to the exon 4/exon 5 boundary may be present within the truncated human midkine protein. For example, 1, 2, 3, 4 or 5 contiguous amino acids of the amino acid sequence set forth in SEQ ID NO: 83 may be present within the truncated human midkine protein, and 1, 2, 3, 4 or 5 contiguous amino acids of the amino acid sequence set forth in SEQ ID NO: 84 may be present or substantially present within the truncated human midkine protein. In one example, the amino acid sequences set forth in SEQ ID NO: 83 and 84 may be present within the truncated human midkine protein.

Alternatively, or in addition, one or more of the amino acids within exon 5 and adjacent to the exon 4/exon 5 boundary may be absent from the truncated midkine protein. For example, 1, 2, 3, 4, 5, 6, 7 or 8 contiguous amino acids of the amino acid sequence set forth in SEQ ID NO: 85 may be absent or substantially absent from the truncated human midkine protein. In some examples, the amino acid sequence set forth in SEQ ID NO: 85 is absent from the truncated human midkine protein.

In one example, the truncated human midkine protein comprises an amino acid sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 78. In another example, the truncated human midkine protein comprises the sequence set forth in SEQ ID NO: 78. In one example, the truncated human midkine protein consists of the sequence set forth in SEQ ID NO: 78.

In some examples, the truncated human midkine protein in which the amino acid sequence corresponding to exon 4 is absent or substantially absent is about 60 to about 80 amino acids in length. For example, the sequence of the truncated human midkine protein may be 60, or 61, or 62, or 63, or 64, or 65, or 66, or 67, or 68, or 69, or 70, or 71, or 72, or 73, or 74, or 75, or 76, or 77, or 78, or 79, or 80 amino acids in length. In one example, the sequence of the truncated human midkine protein may be 67 amino acids in length.

In one example, about 14, about 25, about 34 or about 58 contiguous amino acids of the C-terminus of human midkine protein are absent from the truncated human midkine protein (e.g., SEQ ID NO: 87). In one example, about 14 contiguous amino acids of the C-terminus of human midkine protein are absent from the truncated human midkine protein. For example, 14 contiguous amino acids of the C-terminus of human midkine protein are absent from the truncated human midkine protein. In such examples, the truncated human midkine protein comprises or consists of the sequence set forth in SEQ ID NO: 87. In another example, about 25 contiguous amino acids of the C-terminus of human midkine protein are absent from the truncated human midkine protein. For example, 25 contiguous amino acids of the C-terminus of human midkine protein are absent from the truncated human midkine protein. In such examples, the truncated human midkine protein comprises or consists of the sequence set forth in SEQ ID NO: 89. In a further example, about 34 contiguous amino acids of the C-terminus of human midkine protein are absent from the truncated human midkine protein. For example, 34 contiguous amino acids of the C-terminus of human midkine protein are absent from the truncated human midkine protein. In such examples, the truncated human midkine protein comprises or consists of the sequence set forth in SEQ ID NO: 91. In another example, about 58 contiguous amino acids of the C-terminus of human midkine protein are absent from the truncated human midkine protein. For example, 58 contiguous amino acids of the C-terminus of human midkine protein are absent from the truncated human midkine protein. In such examples, the truncated human midkine protein comprises or consists of the sequence set forth in SEQ ID NO: 95.

In one example, about 1 to 3 contiguous amino acids of the C-terminus of human midkine protein are absent from the truncated human midkine protein. In another example, about 4 to 6 contiguous amino acids of the C-terminus of human midkine protein are absent from the truncated human midkine protein. In another example, about 7 to 9 contiguous amino acids of the C-terminus of human midkine protein are absent from the truncated human midkine protein. In another example, about 10 to 12 contiguous amino acids of the C-terminus of human midkine protein are absent from the truncated human midkine protein. In another example, about 13 to 15 contiguous amino acids of the C-terminus of human midkine protein are absent from the truncated human midkine protein. In another example, about 16 to 18 contiguous amino acids of the C-terminus of human midkine protein are absent from the truncated human midkine protein. In another example, about 19 to 21 contiguous amino acids of the C-terminus of human midkine protein are absent from the truncated human midkine protein. In another example, about 22 to 24 contiguous amino acids of the C-terminus of human midkine protein are absent from the truncated human midkine protein. In another example, about 25 to 27 contiguous amino acids of the C-terminus of human midkine protein are absent from the truncated human midkine protein. In another example, about 30 to 32 contiguous amino acids of the C-terminus of human midkine protein are absent from the truncated human midkine protein. In another example, about 33 to 35 contiguous amino acids of the C-terminus of human midkine protein are absent from the truncated human midkine protein. In another example, about 36 to 38 contiguous amino acids of the C-terminus of human midkine protein are absent from the truncated human midkine protein. In another example, about 48 to 51 contiguous amino acids of the C-terminus of human midkine protein are absent from the truncated human midkine protein. In another example, about 52 to 54 contiguous amino acids of the C-terminus of human midkine protein are absent from the truncated human midkine protein. In another example, about 55 to 57 contiguous amino acids of the C-terminus of human midkine protein are absent from the truncated human midkine protein.

In one example, the truncated human midkine protein comprises an amino acid sequence having at least 80% sequence identity to the sequence set forth in any one of SEQ ID NOs: 87, 89, 91 and 95.

For example, the truncated human midkine protein comprises an amino acid sequence having at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 87. In one example, the truncated human midkine protein comprises an amino acid sequence having the sequence set forth in SEQ ID NO: 87. In another example, the truncated human midkine protein consists of an amino acid sequence having the sequence set forth in SEQ ID NO: 87.

For example, the truncated human midkine protein comprises an amino acid sequence having at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 89. In one example, the truncated human midkine protein comprises an amino acid sequence having the sequence set forth in SEQ ID NO: 89. In another example, the truncated human midkine protein consists of an amino acid sequence having the sequence set forth in SEQ ID NO: 89.

For example, the truncated human midkine protein comprises an amino acid sequence having at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 91. In one example, the truncated human midkine protein comprises an amino acid sequence having the sequence set forth in SEQ ID NO: 91. In another example, the truncated human midkine protein consists of an amino acid sequence having the sequence set forth in SEQ ID NO: 91.

For example, the truncated human midkine protein comprises an amino acid sequence having at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 95. In one example, the truncated human midkine protein comprises an amino acid sequence having the sequence set forth in SEQ ID NO: 95. In another example, the truncated human midkine protein consists of an amino acid sequence having the sequence set forth in SEQ ID NO: 95.

In one example, the composition comprises a polynucleotide encoding the truncated human midkine protein of the disclosure as described herein. In accordance with this example, the sequence corresponding to exon 4 (e.g., the set forth in SEQ ID NO: 71 or 72) is absent or partially absent from the polynucleotide.

In one example, the polynucleotide is an mRNA. In accordance with this example, the sequence set forth in SEQ ID NO: 72 is absent or partially absent from the polynucleotide. For example, at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% of the sequence set forth in SEQ ID NO: 72 may be absent from the polynucleotide encoding the truncated midkine protein. In one example, the polynucleotide encoding the truncated human midkine protein of the disclosure comprises a RNA sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 77. In one example, the polynucleotide encoding the truncated midkine protein comprises the RNA sequence set forth in SEQ ID NO: 77.

In accordance with examples in which one or more amino acids within exon 4 and adjacent to the exon 3/exon 4 boundary are present within the truncated midkine protein of the disclosure, the nucleotides encoding those further amino acids (e.g., nucleotides encoding 1, 2, 3, 4 or 5 contiguous amino acids) may also be present within the polynucleotide sequence encoding the truncated human midkine protein of the disclosure. For example, the polynucleotide sequence set forth in SEQ ID NO: 81 may be present or substantially present within an mRNA sequence encoding the truncated midkine protein of the disclosure. In one example, the sequence set forth in SEQ ID NO: 81 is present within an mRNA sequence encoding the truncated midkine protein of the disclosure.

Alternatively, or in addition, where one or more amino acids within exon 4 which are adjacent to the exon 4/exon 5 boundary are present within the truncated midkine protein of the disclosure, the nucleotides encoding those amino acids (e.g., nucleotides encoding 1, 2, 3, 4 or 5 contiguous amino acids) may also be present within the polynucleotide sequence encoding the truncated human midkine protein of the disclosure. For example, the polynucleotide sequence set forth in SEQ ID NO: 82 may be present or substantially present within an mRNA sequence encoding the truncated midkine protein of the disclosure. In one example, the sequence set forth in SEQ ID NO: 82 is present within an mRNA sequence encoding the truncated midkine protein of the disclosure.

Alternatively, or in addition, where one or more of the amino acids within exon 5 and adjacent to the exon 4/exon 5 boundary are absent from the truncated midkine protein of the disclosure, the nucleotides encoding those further amino acids (e.g., nucleotides encoding 1, 2, 3, 4, 5, 6, 7 or 8 contiguous amino acids) may also be absent from the polynucleotide sequence encoding the truncated human midkine protein of the disclosure.

In one example, the polynucleotide is an mRNA and comprises a sequence of SEQ ID NO: 96 with about 42, 75, 102 or 174 contiguous nucleotide deletions of the C-terminus of human midkine mRNA absent from the truncated human midkine mRNA. For example, the mRNA comprises the sequence set forth in SEQ ID NO: 96 with about 42 contiguous nucleotide deletions of the C-terminus of human midkine mRNA absent from the truncated human midkine mRNA. In another example, the mRNA comprises the sequence set forth in SEQ ID NO: 96 with about 75 contiguous nucleotide deletions of the C-terminus of human midkine mRNA absent from the truncated human midkine mRNA. In a further example, the mRNA comprises the sequence set forth in SEQ ID NO: 96 with about 102 contiguous nucleotide deletions of the C-terminus of human midkine mRNA absent from the truncated human midkine mRNA. In another example, the mRNA comprises the sequence set forth in SEQ ID NO: 96 with about 174 contiguous nucleotide deletions of the C-terminus of human midkine mRNA absent from the truncated human midkine mRNA.

In one example, the polynucleotide is an mRNA encoding a truncated human midkine protein in which about 14 contiguous amino acids of the C-terminus of human midkine protein are absent, wherein the mRNA comprises or consists of a polynucleotide sequence having at least 80% identity to the sequence set forth in SEQ ID NO: 86.

For example, the polynucleotide comprises an mRNA sequence having at least 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 86. In one example, the polynucleotide comprises an mRNA sequence having the sequence set forth in SEQ ID NO: 86. In such examples, the polynucleotide sequence encodes an amino acid sequence set forth in SEQ ID NO: 87.

In one example, the polynucleotide is an mRNA encoding a truncated human midkine protein in which about 25 contiguous amino acids of the C-terminus of human midkine protein are absent, wherein the mRNA comprises or consists of a polynucleotide sequence having at least 80% identity to the sequence set forth in SEQ ID NO: 88.

For example, the polynucleotide comprises an mRNA sequence having at least 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 88. In one example, the polynucleotide comprises an mRNA sequence having the sequence set forth in SEQ ID NO: 88. In such examples, the polynucleotide sequence encodes an amino acid sequence set forth in SEQ ID NO: 89.

In one example, the polynucleotide is an mRNA encoding a truncated human midkine protein in which about 34 contiguous amino acids of the C-terminus of human midkine protein are absent, wherein the mRNA comprises or consists of a polynucleotide sequence having at least 80% identity to the sequence set forth in SEQ ID NO: 90.

For example, the polynucleotide comprises an mRNA sequence having at least 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 90. In one example, the polynucleotide comprises an mRNA sequence having the sequence set forth in SEQ ID NO: 90. In such examples, the polynucleotide sequence encodes an amino acid sequence set forth in SEQ ID NO: 91.

In one example, the polynucleotide is an mRNA encoding a truncated human midkine protein in which about 58 contiguous amino acids of the C-terminus of human midkine protein are absent, wherein the mRNA comprises or consists of a polynucleotide sequence having at least 80% identity to the sequence set forth in SEQ ID NO: 94.

For example, the polynucleotide comprises an mRNA sequence having at least 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 94. In one example, the polynucleotide comprises an mRNA sequence having the sequence set forth in SEQ ID NO: 94. In such examples, the polynucleotide sequence encodes an amino acid sequence set forth in SEQ ID NO: 95.

In another example, the polynucleotide is a DNA sequence. In accordance with this example, the sequence set forth in SEQ ID NO: 71 is absent or partially absent from the polynucleotide. For example, at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% of the sequence set forth in SEQ ID NO: 71 may be absent from the polynucleotide encoding the truncated midkine protein. In one example, the polynucleotide comprises a DNA sequence having at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence set forth in SEQ ID NO: 76. In one example, the polynucleotide encoding the truncated midkine protein comprises the DNA sequence set forth in SEQ ID NO: 76.

In accordance with examples in which one or more amino acids within exon 4 and adjacent to the exon 3/exon 4 boundary are present within the truncated midkine protein of the disclosure, the nucleotides encoding those amino acids (e.g., nucleotides encoding 1, 2, 3, 4 or 5 contiguous amino acids) may also be present within the polynucleotide sequence encoding the truncated human midkine protein of the disclosure. For example, the polynucleotide sequence set forth in SEQ ID NO: 79 may be present or substantially present within DNA sequence encoding the truncated midkine protein of the disclosure. In one example, the sequence set forth in SEQ ID NO: 79 is present within a DNA sequence encoding the truncated midkine protein of the disclosure.

Alternatively, or in addition, where one or more amino acids within exon 4 which are adjacent to the exon 4/exon 5 boundary are present within the truncated midkine protein of the disclosure, the nucleotides encoding those amino acids (e.g., nucleotides encoding 1, 2, 3, 4 or 5 contiguous amino acids) may also be present within the polynucleotide sequence encoding the truncated human midkine protein of the disclosure. For example, the polynucleotide sequence set forth in SEQ ID NO: 80 may be present or substantially present within a DNA sequence encoding the truncated midkine protein of the disclosure. In one example, the sequence set forth in SEQ ID NO: 80 is present within a DNA sequence encoding the truncated midkine protein of the disclosure.

Alternatively, or in addition, where one or more of the amino acids within exon 5 and adjacent to the exon 4/exon 5 boundary are absent from the truncated midkine protein of the disclosure, the nucleotides encoding those amino acids (e.g., nucleotides encoding 1, 2, 3, 4, 5, 6, 7 or 8 contiguous amino acids) may also be absent from the polynucleotide sequence encoding the truncated human midkine protein of the disclosure.

In accordance with any example in which the polynucleotide is a DNA sequence, the DNA sequence may be operably-linked to a promoter and/or comprised within an expression vector. For example, the polynucleotide is a DNA sequence encoding the truncated midkine protein and the polynucleotide is operably-linked to a promoter. In one example, the polynucleotide is a DNA sequence encoding the truncated midkine protein and the polynucleotide is comprised within an expression vector.

In accordance with any example in which the polynucleotide is a DNA sequence, the DNA sequence may be codon optimized.

The compositions of the disclosure may further comprise one or more pharmaceutically acceptable carriers or diluents. In some examples, the composition further comprises a lipid nanoparticle (LNP). In some examples, the polynucleotide is a mRNA which is formulated with a LNP.

In accordance with an example in which the composition comprises an mRNA encoding the truncated midkine protein of the disclosure, the mRNA may be encapsulated in a LNP. Alternatively, the mRNA encoding the truncated midkine protein of the disclosure may be bound to the LNP. For example, the mRNA may be absorbed on the LNP.

In accordance with an example in which the composition comprises a truncated midkine protein of the disclosure, the truncated protein may be encapsulated in a LNP. Alternatively, the truncated midkine protein may be bound to the LNP. For example, the truncated midkine protein may be absorbed on the LNP.

In one example, the LNP further comprises a PEG-lipid, a structural lipid and/or a neutral lipid. For example, the LNP further comprises a PEG-lipid. For example, the LNP further comprises a structural lipid. For example, the LNP further comprises a neutral lipid.

In one example, the LNP comprises an ionisable lipid. For example, the ionisable lipid is a cationic lipid. For example, the ionisable lipid is a zwitterionic lipid.

In one example, the LNP does not comprise an ionisable lipid.

The present disclosure also provides a method for producing the truncated human midkine protein or exosome comprising the truncated human midkine protein described herein, said method comprising culturing a cell in the presence of one or more SSOs as described herein for a time and under conditions sufficient for the cell to produce the truncated human midkine protein. In some examples, the method further comprises recovering the truncated human midkine protein or exosome comprising the truncated human midkine protein from the culture. In some examples, the method further comprises purifying the truncated human midkine protein or exosome comprising the truncated human midkine protein from the culture. In one example, the method comprises culturing the cell in the presence of one or more SSOs selected from the group consisting of SEQ ID NO: 60, 61 and 65.

The present disclosure also provides a method for producing the truncated human midkine protein or exosome comprising the truncated human midkine protein as disclosed herein comprising culturing a cell comprising the polynucleotide encoding the truncated human midkine protein as disclosed herein for a time and under conditions sufficient for the cell to produce the truncated human midkine protein.

The present disclosure also provides a method for producing the truncated human midkine protein as disclosed herein comprising culturing a cell comprising the polynucleotide encoding the truncated human midkine protein as disclosed herein for a time and under conditions sufficient for the cell to produce the truncated human midkine protein.

The present disclosure also provides a method for producing the truncated human midkine protein as disclosed herein comprising culturing a cell comprising a composition as disclosed herein for a time and under conditions sufficient for the cell to produce the truncated human midkine protein.

The present disclosure also provides a method for producing a truncated human midkine protein, comprising transfecting a polynucleotide disclosed herein into a cell under conditions sufficient for the cell to produce the truncated human midkine protein, optionally comprising recovering the truncated human midkine protein from the culture.

In some examples, the method further comprises recovering the truncated human midkine protein from the culture. In some examples, the method further comprises purifying the truncated human midkine protein or exosome comprising the truncated human midkine protein from the culture.

In some examples, inhibition of the interaction between the human midkine and the ligand thereof reduces cell migration.

In some examples, inhibition of the interaction between the human midkine and the ligand thereof reduces cancer cell survival. For example, the cancer is a midkine-related cancer. In one example, the cancer is breast cancer. In another example, the cancer is liver cancer.

The present disclosure also provides a method for inhibiting an interaction between human midkine and a ligand thereof on the surface of or in a cell, said method comprising exposing the cell to the composition described herein.

The present disclosure also provides a method for inhibiting human midkine activity in a cell, said method comprising exposing the cell to the composition described herein.

The present disclosure also provides a method for treating or preventing a midkine-related disease or disorder in a subject in need thereof, said method comprising administering to the subject the composition described herein.

The present disclosure also provides for use of the composition described herein, in the preparation of a medicament for treatment or prevention of a midkine-related disease or disorder selected from an autoimmune disease, cancer, or an inflammatory disease in a subject in need thereof. In some examples, the subject is being treated with or will be treated with a chemotherapeutic agent or immunotherapy.

The present disclosure also provides for use of the composition described herein, to treat or prevent a midkine-related disease or disorder selected from an autoimmune disease, cancer, or an inflammatory disease in a subject in need thereof.

In some examples, the cancer is selected from the group consisting of esophageal cancer, thyroid cancer, urinary bladder cancer, colorectal cancer, cutaneous and uveal melanoma, squamous cell carcinoma, osteosarcoma B-cell malignancies, leukemia, head and neck cancer, gall bladder cancer, stomach cancer, pancreatic cancer, thoracic cancer, liver cancer, lung cancer, breast cancer, neuroblastoma, glioblastoma, uterine cancer, ovarian cancer, prostatic cancer, and Wilms tumor. For example, the cancer is liver cancer. For example, the cancer is breast cancer.

In one example, the subject to which the composition of the disclosure is administered has already received treatment with another therapeutic agent for treating a midkine-related disease or disorder. For example, the subject and/or the midkine-related disease or disorder to be treated may be refractory or resistant to treatment with the other agent known for treating a midkine-related disease or disorder. In one example, the other agent known for treating a midkine-related disease or disorder is a chemotherapeutic agent or immunotherapy.

In another example, the composition of the disclosure is administered in combination with another therapeutic agent known for treating a midkine-related disease or disorder i.e., as an adjunctive therapy.

Treatment of a midkine-related disease or disorder in accordance with any example described herein, may comprise one or more of inhibiting, reducing or preventing midkine activity in the subject and/or reducing severity of symptoms associated with a midkine-related disease or disorder. In one example, the medicament will reduce midkine gene transcription products in the subject to which the medicament is administered.

Examples of midkine-related diseases or disorders that can be inhibited, treated or prevented include, but are not limited to, autoimmune diseases, cancer, or inflammatory diseases. In one example, the midkine-related disease or disorder is cancer. In another example, the midkine-related disease or disorder is an inflammatory disease.

### BRIEF DESCRIPTION OF THE DRAWINGS

FIG. 1 a) shows the SSO nomenclature. SSO name includes information on the gene, species, exon number, and sequence coordinates relative to splice donor and acceptor sites; and b) shows the PCR primer nomenclature. PCR primer ID includes species, gene, target, primer direction and additional information.

FIG. 2 is the midkine (MDK) transcripts reported on Ensembl. Each box represents an exon, and the solid black line represents introns. Chevron sides indicate exons bounded by partial codons. Primers are shown in purple.

FIG. 3 is the Midkine genomic sequence (NC_000011.10) annotated with exon regions numbered based on MANE select transcript T203/transcript variant 3. Primers are shown in purple.

FIG. 4 is the optimisation of six PCR primer sets by alteration of PCR methods, annealing temperature, cycle number, and cell type.

FIG. 5 provides the locations of exon splice enhancer (ESE) and exon splice silencer (ESS) motifs within midkine mRNA predicted by Spliceaid and SSO annealing sites targeted to remove a) exon 3 and b) exon 4. Relative predicted splice factor binding site motif scores are indicated on the y-axis with positive values above the mRNA sequence indicative of splice enhancer motifs while negative values indicate splice silencer motifs. Exonic sequences are shown in upper case letters, and intronic sequences are depicted in lower case. The first generation 2′OMe-PS SSOs are shown in black, the second generation microwalked SSOs in blue and the single 20-mer SSO in purple.

FIG. 6 provides evaluation of SSOs in Huh7 cells. RT-PCR analysis of midkine transcripts after Lipofectamine 3000 transfection with SSOs targeting midkine mRNA a) exon 3 and b) exon 4. Transfection concentrations (50-200 nM) are indicated above the gel image. Relative abundance (%) of amplicons are shown in the graph beside each gel image. GTC, Gene Tools control; Neg, no template PCR control; UT, untreated; bp, base pairs; FL, full-length amplicon; nM, nanomolar; i, intron.

FIG. 7 provides evaluation of two-SSO cocktails in Huh7 cells. RT-PCR analysis of midkine transcripts after Lipofectamine 3000 transfection with two-SSO cocktails targeting midkine mRNA a) exon 3 and b) exon 4. Transfection concentrations are indicated above the gel image. Relative abundance (%) of amplicons are shown in the graph below each gel image. GTC, Gene Tools control; Neg, no template PCR control; UT, untreated; bp, base pairs; FL, full-length amplicon; nM, nanomolar; i, intron.

FIG. 8 provides evaluation of lower SSO concentrations in Huh7 cells. RT-PCR analysis of midkine transcripts after Lipofectamine 3000 transfection with SSOs targeting midkine a) exon 3 and b) exon 4. Transfection concentrations are indicated above the gel image. Relative abundance (%) of amplicons lacking exon 3 (Δ3) or exon 4 (Δ4) are shown below each gel image. GTC, Gene Tools control; Neg, no template PCR control; UT, untreated; bp, base pairs; FL, full-length amplicon; nM, nanomolar.

FIG. 9 provides evaluation of SSOs microwalked around original sequences in Huh7 cells. RT-PCR analysis of midkine transcripts after Lipofectamine 3000 transfection with SSOs targeting midkine a) exon 3 and b) exon 4. The original sequence is shown in black, the microwalked sequences in blue and the 20-mer sequence in purple. Transfection concentrations are indicated above the gel image. Relative abundance (%) of amplicons are shown in the graph below each gel image. GTC, Gene Tools control; Neg, no template PCR control; UT, untreated; bp, base pairs; FL, full-length amplicon; nM, nanomolar.

FIG. 10 provides evaluation of exon 3+4 SSO cocktails in Huh7 cells. RT-PCR analysis of midkine transcripts after Lipofectamine 3000 transfection with cocktails of one SSO targeting each midkine exon 3 and exon 4. Transfection concentrations are indicated above the gel image. Relative abundance (%) of amplicons are shown in the graph below each gel image. GTC, Gene Tools control; Neg, no template PCR control; UT, untreated; bp, base pairs; FL, full-length amplicon; nM, nanomolar; i, intron.

FIG. 11 provides evaluation of SSOs in SHSY5Y cells. RT-PCR analysis of midkine transcripts after Lipofectamine 3000 transfection with individual SSOs targeting midkine a) exon 3 and b) exon 4 or c) two-SSO cocktails. Transfection concentrations are indicated above the gel image. Relative abundance (%) of amplicons are shown in the graph beside each gel image. GTC, Gene Tools control; L3K, Lipofectamine 3000 transfection reagent; Neg, no template PCR control; UT, untreated; bp, base pairs; FL, full-length amplicon; nM, nanomolar; i, intron.

FIG. 12 provides evaluation of promising SSOs in HMC-1 cells. a) RT-PCR analysis of midkine transcripts after Lipofectamine 3000 transfection with the most promising SSO targeting midkine exon 3 and 4. Transfection concentrations are indicated above the gel image. b) The SMN positive control indicates transfection efficiency. Relative abundance (%) of amplicons are shown below each gel image. GTC, Gene Tools control; L3K, Lipofectamine 3000 transfection reagent control; Neg, no template PCR control; UT, untreated; bp, base pairs; FL, full-length amplicon; nM, nanomolar. c) Amplification of HMC-1 RNA treated with ezDNase or left untreated with no reverse transcription step to confirm effective removal of contaminating gDNA.

FIG. 13 provides further evaluation of promising SSOs in HMC-1 cells. a) RT-PCR analysis of midkine transcripts after Lipofectamine 3000 transfection with the most promising SSOs targeting midkine exon 3 and 4. Transfection concentrations are indicated above the gel image. b) The SMN positive control indicates transfection efficiency. Relative abundance (%) of amplicons are shown below each gel image. GTC, Gene Tools control; L3K, Lipofectamine 3000 transfection reagent; Neg, no template PCR control; UT, untreated; bp, base pairs; FL, full-length amplicon; nM, nanomolar.

FIG. 14 provides evaluation of the promising exon 4 microwalked SSOs in HMC-1 cells. a) RT-PCR analysis of midkine transcripts after Lipofectamine 3000 transfection with the most promising SSOs microwalked around the original exon 4 sequences. Transfection concentrations are indicated above the gel image. b) The SMN positive control indicates transfection efficiency. Relative abundance (%) of amplicons are shown below each gel image. GTC, GeneTools control; Neg, no template PCR control; UT, untreated; bp, base pairs; FL, full-length amplicon; nM, nanomolar.

FIG. 15 provides an evaluation of the effects of skipping at the pre-mRNA level of Huh7 cells transfected with the most promising 2′OMe-PS SSOs targeting midkine exon 3 and 4 using Lipofectamine 3000. Transfection concentrations are indicated above the images. Relative abundance (%) of amplicons are shown in the graph below the RT-PCR gel image. GTC, Gene Tools control; L3K, Lipofectamine 3000 transfection reagent; Neg, no template PCR control; UT, untreated; bp, base pairs; FL, full-length amplicon; nM, nanomolar.

FIG. 16 provides an evaluation of the effects of skipping with antisense oligonucleotides synthesised using phosphorodiamidate morpholino (PMO) nucleotide chemistry targeting the most promising regions of midkine Exon 3 and 4 RT-PCR analysis of SHSY5Y cells transfected with one Exon 3 and three Exon 4 PMO SSOs using Neon transfection reagent. Transfection concentrations are indicated above the images. Relative abundance (%) of amplicons are shown in the graph below the RT-PCR gel image. GTC, Gene Tools control; Zap, Neon transfection reagent; UT, untreated; bp, base pairs; FL, full-length amplicon; nM, nanomolar.

FIG. 17 provides further evaluation of SSOs at the protein level. Western blot analysis of Huh7 cell a) SHSY5Y cells b) transfected with the most promising PMO AOs targeting midkine exon 3 and 4 using Lipofectamine 3000. Transfection concentrations are indicated above the images. GTC, Gene Tools control; L3K, lipofectamine 3000 transfection reagent.

FIG. 18 is a depiction of the full length midkine protein sequence overlaid on the mRNA midkine sequence including the 5′- and 3′-UTRs. The 5 Exons are depicted with different colours and the precise sequences at the Exon boundaries shown.

FIG. 19 is a depiction of a Western blot analysis of MDA-MB-231 breast cancer cells transfected with SEQ ID NOs: 86, 88, 90, 92, 94 and 96. Con UT=Control Untransfected; Con LMM=Control Lipofectamine MessengerMax.

FIG. 20 is a depiction of Huh7 cell viability 72 hours post transfection with SEQ ID NOs: 86, 88, 90, 92, 94 and 96. LMM=Control Lipofectamine MessengerMax; FL=Full Length Midkine.

FIG. 21 is a depiction of MDA-MD-231 cell viability 72 hours post transfection with SEQ ID NOs: 86, 88, 90, 92, 94 and 96. LMM=Control Lipofectamine MessengerMax; FL=Full Length Midkine; GFP=Green Fluorescent Protein.

### KEY TO THE SEQUENCE LISTING

SEQ ID NO: 1 hMDK Ex1F
SEQ ID NO: 2 hMDK Ex5R (o)
SEQ ID NO: 3 hMDK Ex5R (i)
SEQ ID NO: 4 hMDK Ex2F (o)
SEQ ID NO: 5 hMDK Ex2F (i)
SEQ ID NO: 6 Homo sapiens midkine full length sequence
SEQ ID NO: 7 Homo sapiens midkine region 1
SEQ ID NO: 8 Homo sapiens midkine region 2
SEQ ID NO: 9 Homo sapiens midkine region 3
SEQ ID NO: 10 Homo sapiens midkine region 4
SEQ ID NO: 11 Homo sapiens midkine region 5
SEQ ID NO: 12 Homo sapiens midkine region 6
SEQ ID NO: 13 Homo sapiens midkine region 7
SEQ ID NO: 14 Homo sapiens midkine region 8
SEQ ID NO: 15 Homo sapiens midkine region 9
SEQ ID NO: 16 Homo sapiens midkine region 10
SEQ ID NO: 17 Homo sapiens midkine region 11
SEQ ID NO: 18 Homo sapiens midkine region 12
SEQ ID NO: 19 Homo sapiens midkine region 13
SEQ ID NO: 20 Homo sapiens midkine region 14
SEQ ID NO: 21 Homo sapiens midkine region 15
SEQ ID NO: 22 Homo sapiens midkine region 16
SEQ ID NO: 23 Homo sapiens midkine region 17
SEQ ID NO: 24 Homo sapiens midkine region 18
SEQ ID NO: 25 Homo sapiens midkine region 19
SEQ ID NO: 26 Homo sapiens midkine region 20
SEQ ID NO: 27 Homo sapiens midkine region 21
SEQ ID NO: 28 Homo sapiens midkine region 22
SEQ ID NO: 29 Homo sapiens midkine region 23
SEQ ID NO: 30 Homo sapiens midkine region 24
SEQ ID NO: 31 Homo sapiens midkine region 25
SEQ ID NO: 32 Homo sapiens midkine region 26
SEQ ID NO: 33 Homo sapiens midkine region 27
SEQ ID NO: 34 Homo sapiens midkine region 28
SEQ ID NO: 35 Homo sapiens midkine region 29
SEQ ID NO: 36 Homo sapiens midkine region 30
SEQ ID NO: 37 Homo sapiens midkine region 31
SEQ ID NO: 38 Homo sapiens midkine region 32
SEQ ID NO: 39 Homo sapiens midkine region 33
SEQ ID NO: 40 Homo sapiens midkine region 34
SEQ ID NO: 41 Homo sapiens midkine region 35
SEQ ID NO: 42 Homo sapiens midkine region 36
SEQ ID NO: 43 MDK H3A(−12+13)
SEQ ID NO: 44 MDK H3A(−04+21)
SEQ ID NO: 45 MDK H3A(+89+113)
SEQ ID NO: 46 MDK H3A(+143+167)
SEQ ID NO: 47 MDK H3A(+146+165)
SEQ ID NO: 48 MDK H3D(+15−10)
SEQ ID NO: 49 MDK H4A(−12+13)
SEQ ID NO: 50 MDK H4A(−04+21)
SEQ ID NO: 51 MDK H4A(+74+98)
SEQ ID NO: 52 MDK H4A(+106+130)
SEQ ID NO: 53 MDK H4D(+10−15)
SEQ ID NO: 54 MDK H3A(+133+157)
SEQ ID NO: 55 MDK H3A(+138+162)
SEQ ID NO: 56 MDK H3D(+21−04)
SEQ ID NO: 57 MDK H3D(+10−15)
SEQ ID NO: 58 MDK H3D(+05−20)
SEQ ID NO: 59 MDK H4A(+69+93)
SEQ ID NO: 60 MDK H4A(+79+103)
SEQ ID NO: 61 MDK H4A(+101+125)
SEQ ID NO: 62 MDK H4A(+111+135)
SEQ ID NO: 63 MDK H4D(+15−10)
SEQ ID NO: 64 MDK H4D(+05−20)
SEQ ID NO: 65 MDK H4A(+100+124)
SEQ ID NO: 66 RNA sequence for Exon 1 of human midkine—5′UTR
SEQ ID NO: 67 RNA sequence for Exon 2 of human midkine
SEQ ID NO: 68 Amino acid sequence corresponding to Exon 2 of human midkine
SEQ ID NO: 69 RNA sequence for Exon 3 of human midkine
SEQ ID NO: 70 Amino acid sequence corresponding to Exon 3 of human midkine
SEQ ID NO: 71 DNA sequence for Exon 4 of human midkine
SEQ ID NO: 72 RNA sequence for Exon 4 of human midkine
SEQ ID NO: 73 Amino acid sequence corresponding to Exon 4 of human midkine
SEQ ID NO: 74 RNA sequence for Exon 5 of human midkine—C-terminus and 3′UTR
SEQ ID NO: 75 Full length coding region midkine protein sequence (excluding signal peptide)
SEQ ID NO: 76 DNA sequence encoding truncated midkine in which exon 4 is absent
SEQ ID NO: 77 mRNA sequence for truncated midkine in which exon 4 is absent
SEQ ID NO: 78 Amino acid sequence for truncated midkine in which sequence corresponding to exon 4 is absent
SEQ ID NO: 79 DNA sequence for 5′ fragment of Exon 4 of human midkine
SEQ ID NO: 80 DNA sequence for 3′ fragment of Exon 4 of human midkine
SEQ ID NO: 81 RNA sequence for 5′ fragment of Exon 4 of human midkine
SEQ ID NO: 82 RNA sequence for 3′ fragment of Exon 4 of human midkine
SEQ ID NO: 83 Amino acid sequence corresponding to 5′ fragment of Exon 4 of human midkine
SEQ ID NO: 84 Amino acid sequence corresponding to 3′ fragment of Exon 4 of human midkine
SEQ ID NO: 85 Amino acid sequence corresponding to Exon 5 of human midkine
SEQ ID NO: 86 RNA sequence corresponding to midkine Δ14
SEQ ID NO: 87 Amino acid sequence corresponding to midkine Δ14
SEQ ID NO: 88 RNA sequence corresponding to midkine Δ25
SEQ ID NO: 89 Amino acid sequence corresponding to midkine Δ25
SEQ ID NO: 90 RNA sequence corresponding to midkine Δ34
SEQ ID NO: 91 Amino acid sequence corresponding to midkine Δ34
SEQ ID NO: 92 RNA sequence corresponding to midkine Δ41
SEQ ID NO: 93 Amino acid sequence corresponding to midkine Δ41
SEQ ID NO: 94 RNA sequence corresponding to midkine Δ58
SEQ ID NO: 95 Amino acid sequence corresponding to midkine Δ58
SEQ ID NO: 96 RNA sequence corresponding to the coding region of the full length midkine including the natural start codon and peptide leader sequence.

### DETAILED DESCRIPTION

### General Techniques and Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular biology, immunology, immunohistochemistry, protein chemistry, and biochemistry).

Unless otherwise indicated, the recombinant protein, cell culture, molecular biology, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The words “a” and “an” when used in this disclosure, including the claims, denotes one or more.

As used herein, the terms “about” and “approximately” are interchangeable, and should generally be understood to refer to a range of numbers around a given number, as well as to all numbers in a recited range of numbers. Moreover, all numerical ranges herein should be understood to include each whole integer within the range. Unless stated to the contrary, the terms “about” and “approximately” refers to +/−10%, more preferably +/−5%, more preferably +/−1%, of the designated value. As used herein in the context of a truncated human midkine protein sequence, “about” will be understood to refer to one amino acid either side of the recited number.

The terms “e.g.,” and “i.e.” as used herein, is used merely by way of example, without limitation intended, and should not be construed as referring only those items explicitly enumerated in the disclosure.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Truncated Midkine Proteins

As described herein, the present disclosure provides a truncated human midkine protein in which the amino acid sequence encoded by exon 4 and/or exon 5 of human midkine is absent, partially absent or substantially absent. The truncated forms of midkine protein of the disclosure are non-functional but retain the ability to form heterodimers with full-length functional midkine protein. In this way, the truncated midkine protein is able to form a heterodimer with full-length functional midkine protein and inhibit the activity of the full length human midkine in a cell or tissue and/or inhibit the interaction between human midkine and a ligand thereof on the surface of, or in, a cell.

Reference herein to a “human midkine protein”, “human MDK protein” or similar shall be understood to mean native, full length forms of human midkine protein. For the purposes of nomenclature only and not limitation, an exemplary sequence of a human midkine protein is set out in NCBI Reference Sequence: BC011704.2 (and set out in SEQ ID NO: 6).

The term “truncated” as used in the context of midkine protein refers to a midkine protein in which a portion of the protein is absent such that the protein length is shortened relative to the native, full length human midkine protein. Accordingly, it will be understood that a truncated protein contains less than the complete number of amino acids found in a native protein. Whilst the present disclosure is not limited to any particular specific length of truncated protein, it is contemplated that the amino acid sequence encoded by exon 4 of human midkine is absent, or partially or substantially absent from the truncated form. In this regard, it is contemplated that the present disclosure may encompass any length of a truncated midkine protein in which the amino acid sequence encoded by exon 4 of human midkine is absent, partially absent or substantially absent, provided that the truncated protein interferes with activity of the full length native midkine protein (e.g., by way of forming a heterodimer therewith or otherwise inhibiting native midkine from binding to, or interacting with, a ligand).

As used herein, the term “native protein” refers to the protein inside or on the surface of a cell that is in its native or natural state and unaltered, is properly folded, assembled and/or secreted, and is operative and functional.

The term “substantially absent” as used in the context of a midkine protein sequence of the disclosure shall be understood to mean that almost all of the sequence referred to is absent. For example, a truncated midkine protein in which the amino acid sequence encoded by exon 4 of human midkine is substantially absent comprises less than 30% of the amino acid sequence encoded by exon 4 of human midkine. For example, a truncated midkine protein in which the amino acid sequence encoded by exon 4 of human midkine is substantially absent comprises less than 20% of the amino acid sequence encoded by exon 4 of human midkine. Preferably a truncated midkine protein in which the amino acid sequence encoded by exon 4 of human midkine is substantially absent comprises less than about 10% of the amino acid sequence encoded by exon 4 of human midkine. For example, a truncated midkine protein in which the amino acid sequence encoded by exon 4 of human midkine is substantially absent may comprises fewer than 10 (e.g., fewer than 9, or 8, or 7, or 6, or 5, or 4, or 3, or 2 or 1) of the amino acids encoded by exon 4 of human midkine. Preferably, a truncated midkine protein in which the amino acid sequence encoded by exon 4 of human midkine is substantially absent is non-functional.

The term “partially absent” as used in the context of a midkine protein sequence of the disclosure shall be understood to mean that a portion of the sequence referred to is absent. For example, a truncated midkine protein in which the amino acid sequence encoded by exon 4 of human midkine is partially absent comprises about 60% of the amino acid sequence encoded by exon 4 of human midkine. For example, a truncated midkine protein in which the amino acid sequence encoded by exon 4 of human midkine is partially absent comprises about 70% of the amino acid sequence encoded by exon 4 of human midkine. In another example, a truncated midkine protein in which the amino acid sequence encoded by exon 4 of human midkine is partially absent comprises about 80% of the amino acid sequence encoded by exon 4 of human midkine. In another example, a truncated midkine protein in which the amino acid sequence encoded by exon 4 of human midkine is partially absent comprises about 90% of the amino acid sequence encoded by exon 4 of human midkine. For example, a truncated midkine protein in which the amino acid sequence encoded by exon 4 of human midkine is partially absent may comprises fewer than 10 (e.g., fewer than 9, or 8, or 7, or 6, or 5, or 4, or 3, or 2 or 1) of the amino acids encoded by exon 4 of human midkine. Preferably, a truncated midkine protein in which the amino acid sequence encoded by exon 4 of human midkine is partially absent is non-functional.

The term “substantially present” as used in the context of a midkine protein sequence of the disclosure shall be understood to mean that almost all of the sequence referred to is present.

As described herein, the amino acid sequence encoded by exon 4 of human midkine may be completely absent from the truncated midkine protein of the disclosure. In some examples, the amino acid sequence encoded by exon 5 of human midkine may be absent from the truncated midkine protein of the disclosure. In such examples, the truncated midkine proteins of the disclosure may be designed such that they have no C-terminal amino acids. In accordance with such examples, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 43, about 44, about 45, about 46, about 47, about 48, about 49, about 50, about 51, about 52, about 53, about 54, about 55, about 56, about 57, about 58 or about 59 contiguous amino acids of the C-terminus of human midkine protein are absent from the truncated human midkine protein. In one example, about 14 contiguous amino acids of the C-terminus of human midkine protein are absent from the truncated human midkine protein. In such examples, the truncated human midkine protein comprises an amino acid sequence set forth in SEQ ID NO: 87. In another example, about 25 contiguous amino acids of the C-terminus of human midkine protein are absent from the truncated human midkine protein.

In such examples, the truncated human midkine protein comprises an amino acid sequence set forth in SEQ ID NO: 89. In other examples, about 34 contiguous amino acids of the C-terminus of human midkine protein are absent from the truncated human midkine protein. In such examples, the truncated human midkine protein comprises an amino acid sequence set forth in SEQ ID NO: 91. In one example, about 58 contiguous amino acids of the C-terminus of human midkine protein are absent from the truncated human midkine protein. In such examples, the truncated human midkine protein comprises an amino acid sequence set forth in SEQ ID NO: 95.

The amino acid sequence encoded by exon 4 of human midkine is set forth in SEQ ID NO: 73. In accordance with certain examples in which the amino acid sequence encoded by exon 4 of human midkine protein is substantially absent, at least about 70% (e.g., at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) of the sequence set forth in SEQ ID NO: 73 is absent from the truncated human midkine protein. In another example, the amino acid sequence encoded by exon 4 of human midkine protein (i.e., the amino acid sequence set forth in SEQ ID NO: 73) is absent from the truncated human midkine protein.

In accordance examples in which the amino acid sequence encoded by exon 4 is substantially absent, one or more amino acids within exon 4 and adjacent to the exon 3/exon 4 boundary may also be present within the truncated midkine protein. For example, 1, 2, 3, 4 or 5 contiguous amino acids of the amino acid sequence set forth in SEQ ID NO: 83 may be present within the truncated human midkine protein. In some examples, the amino acid sequence set forth in SEQ ID NO: 83 is present within the truncated human midkine protein. Alternatively, or in addition, where the amino acid sequence encoded by exon 4 is substantially absent, one or more amino acids within exon 4 which are adjacent to the exon 4/exon 5 boundary may be present. For example, 1, 2, 3, 4 or 5 contiguous amino acids of the amino acid sequence set forth in SEQ ID NO: 84 may be present within the truncated human midkine protein. In yet another example, one or more amino acids within exon 4 and adjacent to the exon 3/exon 4 boundary may also be present within the truncated midkine protein, and one or more amino acids within exon 4 which are adjacent to the exon 4/exon 5 boundary may be present within the truncated human midkine protein. For example, 1, 2, 3, 4 or 5 contiguous amino acids of the amino acid sequence set forth in SEQ ID NO: 83 may be a present within the truncated human midkine protein, and 1, 2, 3, 4 or 5 contiguous amino acids of the amino acid sequence set forth in SEQ ID NO: 84 may be present within the truncated human midkine protein. In one example, the amino acid sequences set forth in SEQ ID NO: 83 and 84 may be present within the truncated human midkine protein.

Alternatively, or in addition, one or more of the amino acids within exon 5 and adjacent to the exon 4/exon 5 boundary may be absent from the truncated midkine protein. For example, 1, 2, 3, 4, 5, 6, 7 or 8 contiguous amino acids of the amino acid sequence set forth in SEQ ID NO: 85 may be absent, partially absent or substantially absent from the truncated human midkine protein. In some examples, the amino acid sequence set forth in SEQ ID NO: 85 is absent from the truncated human midkine protein.

In one example, the truncated human midkine protein comprises an amino acid sequence having at least about 70% (e.g., at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) sequence identity to the sequence set forth in SEQ ID NO: 78. In another example, the truncated human midkine protein comprises the sequence set forth in SEQ ID NO: 78. In one example, the truncated human midkine protein consists of the sequence set forth in SEQ ID NO: 78.

In some examples, the truncated human midkine protein in which the amino acid sequence corresponding to exon 4 is absent, partially absent or substantially absent is about 60 to about 80 amino acids in length. For example, the sequence of the truncated human midkine protein may be 60, or 61, or 62, or 63, or 64, or 65, or 66, or 67, or 68, or 69, or 70, or 71, or 72, or 73, or 74, or 75, or 76, or 77, or 78, or 79, or 80 amino acids in length. In one example, the sequence of the truncated human midkine protein may be 67 amino acids in length.

### Production of Polypeptides

The truncated midkine proteins of the disclosure may be produced by any means in the art known for producing polypeptides.

In one example, truncated midkine proteins of the disclosure are synthesised using any chemical method known to the skilled artisan. For example, synthetic proteins may be prepared using known techniques of solid phase, liquid phase, or peptide condensation, or any combination thereof, and can include natural and/or unnatural amino acids.

In another example, the truncated midkine proteins of the disclosure may be expressed by recombinant means. For example, a nucleic acid encoding a truncated midkine protein may be placed in operable connection with a promoter or other regulatory sequence capable of regulating expression in cellular system or organism.

Typical promoters suitable for expression in bacterial cells include, for example, the lacz promoter, the Ipp promoter, temperature-sensitive XL or XR promoters, T7 promoter, T3 promoter, SP6 promoter or semi-artificial promoters such as the IPTG-inducible tac promoter or lacUV5 promoter. A number of other gene construct systems for expressing nucleic acids encoding truncated midkine proteins of the disclosure in bacterial cells are well-known in the art and are described, for example, in Ausubel et al. (1988), and Sambrook et al. (2001).

Numerous expression vectors for expression of recombinant polypeptides in bacterial cells have been described, and include, for example, PKC3, pKK173-3, pET28, the pCR vector suite (Invitrogen), pGEM-T Easy vectors (Promega), the pL expression vector suite (Invitrogen) or pBAD/thio-TOPO series of vectors containing an arabinose-inducible promoter (Invitrogen), amongst others.

Typical promoters suitable for expression in yeast cells such as, for example, a yeast cell selected from the group comprising Pichia pastoris, Saccharomyces cerevisiae and Schizosaccharomyces pombe, include, but are not limited to, the ADH1 promoter, the GAL1 promoter, the GAL4 promoter, the CUP1 promoter, the PH05 promoter, the nmt promoter, the RPR1 promoter, or the TEF1 promoter.

Expression vectors for expression in yeast cells include, for example, the pACT vector (Clontech), the pDBleu-X vector, the pPIC vector suite (Invitrogen), the pGAPZ vector suite (Invitrogen), the pHYB vector (Invitrogen), the pYD 1 vector (Invitrogen), and the pNMT 1, pNMT41, pNMT81 TOPO vectors (Invitrogen), the pPC86-Y vector (Invitrogen), the pRH series of vectors (Invitrogen), pYESTrp series of vectors (Invitrogen).

Expression vectors for expression in mammalian cells include, for example, the pcDNA vector suite (Invitrogen), the pTARGET series of vectors (Promega), and the pSV vector suite (Promega). However, numerous other expression vectors for mammalian cells are known in the art and contemplated herein.

Suitable methods for transforming and transfecting host cells can be found in Sambrook et al. 2001 and other laboratory textbooks. In one example, a nucleic acid encoding the truncated midkine protein of the disclosure may be introduced into prokaryotic cells using for example, electroporation or calcium-chloride mediated transformation. In another example, nucleic acid may be introduced into mammalian cells using, for example, microinjection, calcium phosphate or calcium chloride co-precipitation, DEAE-dextran mediated transfection, transfection mediated by liposomes such as by using Lipofectamine (Invitrogen) and/or cellfectin (Invitrogen), PEG mediated DNA uptake, electroporation, transduction by Adenoviuses, Herpesviruses, Togaviruses or Retroviruses and microparticle bombardment such as by using DNA-coated tungsten or gold particles. Alternatively, a nucleic acid may be introduced into yeast cells using conventional techniques such as, for example, electroporation, and PEG mediated transformation.

In yet another example, the truncated midkine proteins of the disclosure may be expressed by cell or organism into which splice switching oligonucleotides of the disclosure have been introduced such that the truncated form of midkine is produced by the cell or organism. The truncated midkine protein may then be recovered.

Thus, the present disclosure also contemplates a method of producing truncated midkine proteins by the use of splice-switching oligonucleotides (SSO) targeting the pre-mRNA sequence of human midkine.

For example, the present disclosure provides compositions comprising splice-switching oligonucleotides (SSO) targeting the pre-mRNA sequence of human midkine.

In some examples, the SSOs as described herein can be used to produce a truncated human midkine protein as described herein. In one example, the SSOs can be used to produce a truncated human midkine protein as described herein in vitro. For example, the disclosure comprises providing a SSO as described herein to an in vitro cell to produce the truncated human midkine protein, and optionally isolating the truncated human midkine protein from the cell.

In another example, the SSOs can be used to produce a truncated human midkine protein as described herein in vivo. For example, the disclosure comprises providing an SSO to an in vivo cell or a subject to produce a truncated human midkine protein as described herein in vivo.

An SSO of the disclosure is between 10 and 50 nucleotides in length (e.g., between 20-25 nucleotides in length), and comprises a polynucleotide sequence of at least 10 contiguous nucleotides which is substantially complementary to a target region of corresponding length within a pre-mRNA sequence of human midkine.

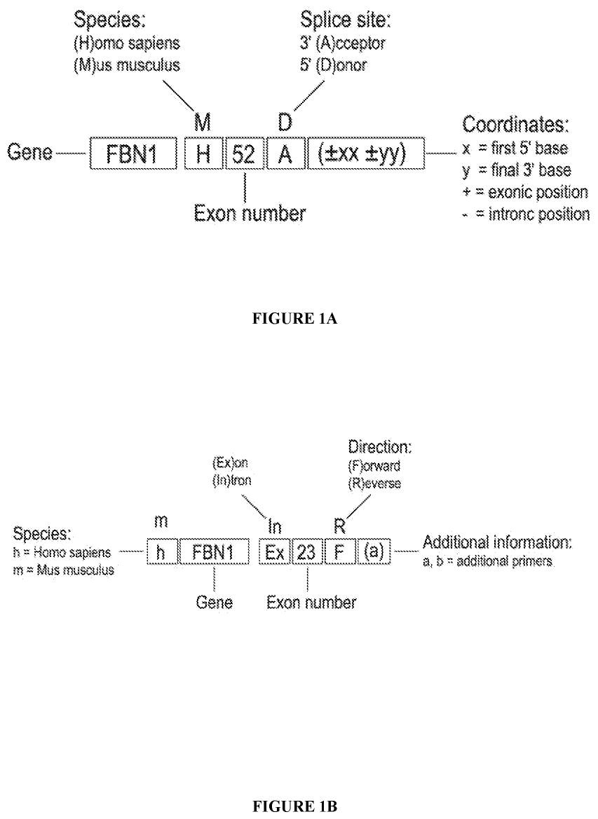

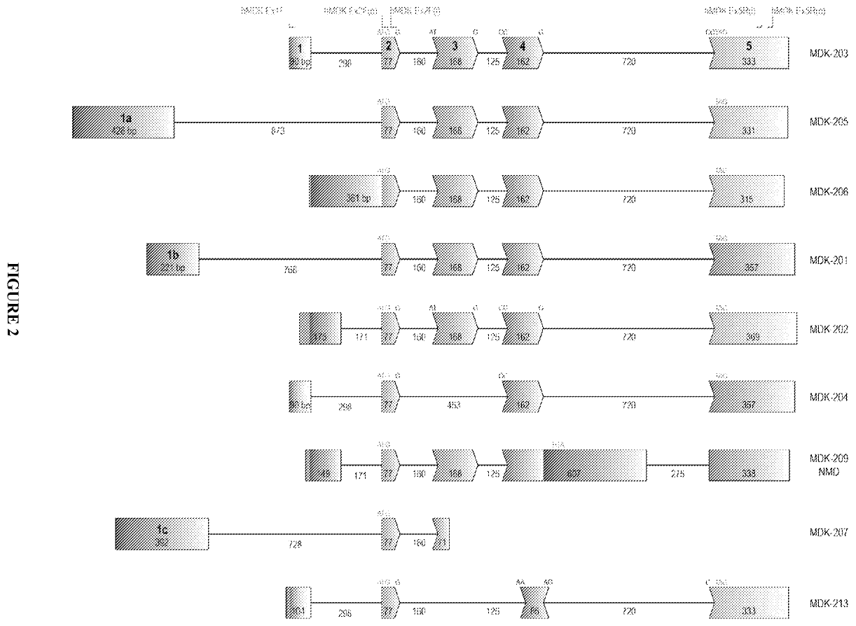

As used herein, the terms “splice-switching oligonucleotide”, “splice switching oligomer”, “SSO”, and “antisense oligonucleotide”, or “AO” when used in the context of splice switching, refers to a short oligonucleotide that is substantially complementary to, and able to base-pair with, a fragment of a pre-mRNA sequence and thereby disrupt the normal splicing repertoire of the transcript by blocking the RNA-RNA base-pairing or protein-RNA binding interactions that occur between components of the splicing machinery and the pre-mRNA. In doing so, a splice-switching oligonucleotide is able to induce targeted exon skipping. The use of SSOs for targeted exon skipping is well known in the art [see for example, G. Hartmann and S. Endres, Manual of Antisense Methodology, Kluwer (1999)]. AO nomenclature system was proposed and published (Aung-Htut M T et al 2019 Int J Mol Sci 20:5030) to distinguish between the different antisense molecules (see FIG. 1).

The term “oligonucleotide” as used in the context of SSOs refers to an oligomer or polymer of ribonucleic acid (RNA) or deoxyribonucleic acid (DNA), wherein the polymer or oligomer of monomers contains any combination ofs or nucleosides, modifieds or nucleosides, sugars, modified sugars, phosphate bridges, or modified phosphorus atom bridges (also referred to herein as “inter-nucleotidic linkage”). Oligonucleotides can be single-stranded or double-stranded or a combination thereof. A single-stranded oligonucleotide can have double-stranded regions and a double-stranded oligonucleotide can have single-stranded regions (such as a microRNA or shRNA).

“RNA” as described herein is meant as a sequence comprising at least one ribonucleotide residue. By “ribonucleotide” is meant a with a hydroxyl group at the 2′ position of a β-D-ribo-furanose moiety. The terms include double-stranded RNA, single-stranded RNA, isolated RNA, such as, messenger RNA as well as altered RNA that differs from naturally occurring RNA by the addition, deletion, substitution and/or alteration of one or mores in the RNA sequences of the instant disclosure can also comprise non-standards, such as non-naturally occurring or chemically synthesised or deoxynucleotides.

The SSO and the DNA or RNA are complementary to each other when a sufficient number of corresponding positions in each sequence are occupied by polynucleotides which can hydrogen bond with each other. Thus, “specifically hybridisable” and “complementary” are terms which are used to indicate a sufficient degree of complementarity or precise pairing such that stable and specific binding occurs between the oligonucleotide and the DNA or RNA target. It is understood in the art that the sequence of an SSO need not be 100% complementary to that of its target region to be specifically hybridisable. An SSO is specifically hybridisable when binding of the compound to the target DNA or RNA sequence interferes with the normal function of the target DNA or RNA, and there is a sufficient degree of complementarity to avoid non-specific binding of the SSO to non-target regions under conditions in which specific binding is desired, i.e., under physiological conditions in the case of in vivo assays or therapeutic treatment, and in the case of in vitro assays, under conditions in which the assays are performed.

As used herein, the term “complementary” with regard to a sequence refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified fragment or length of another sequence. One of skill in the art will recognize that U may be present in RNA, and that T may be present in DNA. Therefore, an A within either of a RNA or DNA sequence may pair with a U in a RNA sequence or T in a DNA sequence.

As used herein, the term “substantially complementary” is used to indicate a sufficient degree of complementarity or precise pairing such that stable and specific binding occurs between nucleic acid sequences e.g., between the SSO and the SSO complement sequence or between the SSO and the target region. That is, the SSO is able to “specifically hybridise” to its cognate target region. It is understood that the sequence of a nucleic acid need not be 100% complementary to that of its target or complement. The term encompasses a sequence complementary to another sequence with the exception of an overhang. In some cases, the sequence is complementary to the other sequence with the exception of 1 to 4 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4 mismatches.

The SSO may be capable of hybridising to its target region under physiological conditions i.e., under normal conditions in a cell.

As used herein, a “target” or “target region” refers to a stretch of polynucleotides within a pre-mRNA sequence of human midkine to which an SSO of the disclosure is substantially complementary (or complementary) and able to hybridise (e.g., under physiological conditions).

The length of an SSO of the disclosure may vary so long as it is capable of binding selectively to the intended location within the pre-mRNA sequence. In this regard, it is well known by those skilled in the art that it is possible to increase or decrease the length of a SSO and/or introduce mismatch bases without eliminating activity. Methods to determine desired activity are disclosed herein and well known to those skilled in the art.

Generally, the SSO will comprise a polynucleotide sequence from about 10 polynucleotides in length up to about 50 polynucleotides in length. For example, the SSO may be about 10 polynucleotides in length, or about 15 polynucleotides in length, or about 20 polynucleotides in length, or about 25 polynucleotides in length, or about 30 polynucleotides in length, or about 35 polynucleotides in length, or about 40 polynucleotides in length, or about 45 polynucleotides in length, or about 50 polynucleotides in length. In particular examples, the length of the SSO is between 15 to 30 polynucleotides in length, such as 15 to 25 polynucleotides in length.

SSOs of the disclosure may be designed to disrupt splicing and thereby induce exon skipping of exon 1, 2, 3, 4 or 5 of human midkine.

As used herein “exon skipping” refers to altering the processing of a pre-mRNA transcript such that the spliced mRNA sequence contains a different combination of exons as a result of exon skipping. In the context of the present disclosure, exon skipping refers to altering splicing of midkine pre-mRNA to achieve a deletion of one or more exons.

In one example, an SSO of the disclosure is designed to specifically hybridise to a target region of corresponding length within the pre-mRNA sequence of human midkine to disrupt splicing and thereby induce exon skipping of exon 3. The degree of identity of the SSO polynucleotide sequence to the target region should be at least 85%, 90%, 95% or 100%. The SSO may of course comprise unrelated sequences which may function to stabilize the sequence such as described herein.

In accordance with this example, the SSO may comprise a polynucleotide sequence of at least 10 contiguous polynucleotides (e.g., 10, or 11, or 12, or 13, or 14, or 15, or 16, or 17, or 18, o 19, or 20, or 21, or 22, or 23, or 24, or 25 contiguous polynucleotides) which is complementary to a region of corresponding length in a sequence set forth in any one of SEQ ID NOs: 7-14, 20-25 and 31-35, optionally with the exception of 1, 2, 3 or 4 mismatches.

In one example, an SSO of the disclosure is designed to specifically hybridise to a target region of corresponding length within the pre-mRNA sequence of human midkine to disrupt splicing and thereby induce exon skipping of exon 4. In accordance with this example, the SSO may comprise a polynucleotide sequence of at least 10 contiguous polynucleotides (e.g., 10, or 11, or 12, or 13, or 14, or 15, or 16, or 17, or 18, o 19, or 20, or 21, or 22, or 23, or 24, or 25 contiguous polynucleotides) which is substantially complementary to a region of corresponding length in a sequence set forth in any one of SEQ ID NOs: 15-19, 26-30 and 36-42. For example, the SSO may comprise a polynucleotide sequence of at least 10 contiguous polynucleotides (e.g., 10, or 11, or 12, or 13, or 14, or 15, or 16, or 17, or 18, o 19, or 20, or 21, or 22, or 23, or 24, or 25 contiguous polynucleotides) which is complementary to a region of corresponding length in a sequence set forth in any one of SEQ ID NOs: 15-19, 26-30 and 36-42, optionally with the exception of 1, 2, 3 or 4 mismatches.

Typically, an SSO of the disclosure will be synthesized in vitro. Methods of synthesizing oligonucleotides are known in the art. However, in some instances where modified polynucleotides and backbones are not required, the SSOs of the disclosure may be expressed in vitro or in vivo in a suitable system, such as by a recombinant virus or cell.

Following production/expression/synthesis, a truncated midkine protein of the present disclosure can be recovered and/or purified using a method known in the art. For example, affinity purification may be used to purify any protein of the present disclosure. Methods for isolating a protein using affinity chromatography are known in the art and described, for example, in Scopes (In: Protein purification: principles and practice, Third Edition, Springer Verlag, 1994).

The degree of purity any protein of the present disclosure may be determined by various methods, including identification of a major large peak on HPLC OR UPLC.

### Polynucleotides

The present disclosure also provides a polynucleotide encoding a truncated human midkine protein described herein.

The term “polynucleotide” as used herein refers to an oligomer or polymer of ribonucleic acid (RNA) or deoxyribonucleic acid (DNA), wherein the polymer or oligomer of polynucleotide monomers contains any combination of polynucleotides or nucleosides, modified polynucleotides or nucleosides, sugars, modified sugars, phosphate bridges, or modified phosphorus atom bridges (also referred to herein as “inter-nucleotidic linkage”). The term “polynucleotide” is used interchangeably herein with the terms “nucleic acid” and “oligonucleotide”. The term “recombinant polynucleotide” refers to a polynucleotide comprising a nucleic acid sequence produced, or which is arrived at, by recombinant means.

As used herein, the term “encode”, “encodes” or “encoding” refers to a region of a RNA (e.g., mRNA) capable of undergoing translation into a polypeptide or protein, or, when used in the context of a DNA, a region of DNA capable of undergoing transcription to produce an mRNA which is capable of being translated into a polypeptide or protein.

As described herein, the present disclosure relates to a polynucleotide encoding the truncated human midkine protein of the disclosure (e.g., wherein the sequence corresponding to exon 4 and/or exon 5 is absent, partially absent or substantially absent from the polynucleotide). The DNA sequence corresponding to exon 4 of human midkine protein is set forth in SEQ ID NO: 71. The RNA sequence corresponding to exon 4 of human midkine protein is set forth in SEQ ID NO: 72.

In one example, the polynucleotide is an mRNA. For example, the mRNA may be a conventional mRNA (cRNA) or a self-amplifying RNA (sa-mRNA). In accordance with examples where the polynucleotide is an mRNA, the sequence set forth in SEQ ID NO: 72 is absent, partially absent or substantially absent from the polynucleotide. For example, at least about 5% (e.g., at least about 10%, or at least about 15%, or at least about 20%, or at least about 25%, or at least about 30%, or at least about 35%, or at least about 40%, or at least about 45%, or at least about 50%) of the sequence set forth in SEQ ID NO: 72 may be absent from the polynucleotide encoding the truncated midkine protein.

The polynucleotide encoding the truncated human midkine protein may comprise an mRNA sequence having at least about 70%, (e.g., at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) sequence identity to the sequence set forth in SEQ ID NO: 77. In one example, the polynucleotide encoding the truncated midkine protein comprises the RNA sequence set forth in SEQ ID NO: 77.

In accordance with examples in which one or more amino acids encoded by exon 4 (adjacent to the exon 3/exon 4 boundary) are present within the truncated midkine protein i.e., where the exon 4 is partially absent or substantially absent from the coding sequence, the nucleotides encoding those amino acids (e.g., nucleotides encoding 1, 2, 3, 4 or 5 contiguous amino acids) may also be present within the polynucleotide sequence encoding the truncated human midkine protein of the disclosure. For example, the polynucleotide sequence set forth in SEQ ID NO: 81 may be present or substantially present within an mRNA sequence encoding the truncated midkine protein of the disclosure. In one example, the sequence set forth in SEQ ID NO: 81 is present within an mRNA sequence encoding the truncated midkine protein of the disclosure.

Alternatively, or in addition, where one or more amino acids within exon 4 which are adjacent to the exon 4/exon 5 boundary are present within the truncated midkine protein of the disclosure, the nucleotides encoding those amino acids (e.g., nucleotides encoding 1, 2, 3, 4 or 5 contiguous amino acids) may also be present within the polynucleotide sequence encoding the truncated human midkine protein of the disclosure. For example, the polynucleotide sequence set forth in SEQ ID NO: 82 may be present or substantially present within an mRNA sequence encoding the truncated midkine protein of the disclosure. In one example, the sequence set forth in SEQ ID NO: 82 is present within an mRNA sequence encoding the truncated midkine protein of the disclosure.

Alternatively, or in addition, where one or more of the amino acids within exon 5 and adjacent to the exon 4/exon 5 boundary are absent from the truncated midkine protein of the disclosure, the nucleotides encoding those further amino acids (e.g., nucleotides encoding 1, 2, 3, 4, 5, 6, 7 or 8 contiguous amino acids) may also be absent from the polynucleotide sequence encoding the truncated human midkine protein of the disclosure. In some examples, exon 5 of human midkine is absent from the polynucleotide encoding the truncated midkine protein.

In accordance with such examples, the polynucleotide encodes a truncated human midkine protein comprising an amino acid sequence set forth in SEQ ID NO: 87. In another example, the polynucleotide encodes a truncated human midkine protein comprising an amino acid sequence set forth in SEQ ID NO: 89. In one example, the truncated human midkine protein comprises an amino acid sequence set forth in SEQ ID NO: 91. In one example, polynucleotide encodes a truncated human midkine protein comprising an amino acid sequence set forth in SEQ ID NO: 95.

In other examples, the polynucleotide is a DNA sequence. In accordance with examples in which the polynucleotide is a DNA sequence, the sequence set forth in SEQ ID NO: 71 is absent, partially absent or substantially absent from the polynucleotide. For example, at least about 70%, (e.g., at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) of the sequence set forth in SEQ ID NO: 71 may be absent from the polynucleotide encoding the truncated midkine protein. In one example, the polynucleotide comprises a DNA sequence having at least about 70% (e.g., at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%) sequence identity to the sequence set forth in SEQ ID NO: 76. In one example, the polynucleotide encoding the truncated midkine protein comprises the DNA sequence set forth in SEQ ID NO: 76.

In accordance with examples in which one or more amino acids within exon 4 and adjacent to the exon 3/exon 4 boundary are present within the truncated midkine protein of the disclosure i.e., where the exon 4 is partially absent or substantially absent from the coding sequence, the nucleotides encoding those amino acids (e.g., nucleotides encoding 1, 2, 3, 4 or 5 contiguous amino acids) may also be present within the polynucleotide sequence encoding the truncated human midkine protein of the disclosure. For example, the polynucleotide sequence set forth in SEQ ID NO: 79 may be present or substantially present within DNA sequence encoding the truncated midkine protein of the disclosure. In one example, the sequence set forth in SEQ ID NO: 79 is present within a DNA sequence encoding the truncated midkine protein of the disclosure.

Alternatively, or in addition, where one or more amino acids within exon 4 which are adjacent to the exon 4/exon 5 boundary are present within the truncated midkine protein of the disclosure, the nucleotides encoding those amino acids (e.g., nucleotides encoding 1, 2, 3, 4 or 5 contiguous amino acids) may also be present within the polynucleotide sequence encoding the truncated human midkine protein of the disclosure. For example, the polynucleotide sequence set forth in SEQ ID NO: 80 may be present or substantially present within a DNA sequence encoding the truncated midkine protein of the disclosure. In one example, the sequence set forth in SEQ ID NO: 80 is present within a DNA sequence encoding the truncated midkine protein of the disclosure.

Alternatively, or in addition, where one or more of the amino acids within exon 5 and adjacent to the exon 4/exon 5 boundary are absent from the truncated midkine protein of the disclosure, the nucleotides encoding those further amino acids (e.g., nucleotides encoding 1, 2, 3, 4, 5, 6, 7 or 8 contiguous amino acids) may also be absent from the polynucleotide sequence encoding the truncated human midkine protein of the disclosure.

In accordance with any example in which the polynucleotide is a DNA sequence, the DNA sequence may be operably-linked to a promoter and/or comprised within an expression vector.

The DNA sequence can be a heterologous DNA sequence. The DNA sequence can include at least one DNA sequence or one or more heterologous DNA sequences.

The DNA sequence can be an optimised DNA sequence. Such optimisation can increase the expression of and in particular, the biological effect (including neutralising effect) of the truncated midkine protein. Optimisation can also improve transcription and/or translation.

Optimisation can include one or more of the following: low GC content leader sequence to increase transcription; mRNA secondary structure reduction and codon optimization; optimal 5′UTR and 3′UTR; optimised transcriptional termination signal; optimal Kozak sequence (e.g., GCC ACC) for increased translation efficiency; and eliminating to the extent possible of cis-acting sequence motifs (e.g. internal TATA boxes).

As used herein, the term “expression vector” refers to a vector comprising a recombinant polynucleotide comprising expression control sequences operatively linked to a polynucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, adenoviruses, and adeno-associated viruses) that incorporate the recombinant polynucleotide.

As used herein, the term “promoter” refers to a DNA element or sequence which is capable of controlling transcription of the polynucleotide of the disclosure into mRNA when the promoter is placed at the 5′ end of (i.e., precedes) the polynucleotide sequence. Thus, a promoter is typically located 5′ (i.e., upstream) of a polynucleotide sequence whose transcription into mRNA it controls, and provides a site for specific binding by RNA polymerase and for initiation of transcription.

As used herein, the term “operably-linked” or “operable linkage” (or similar) means that a coding nucleic acid sequence (i.e., the polynucleotide encoding the truncated midkine protein) is linked to, or in association with, a regulatory sequence, e.g., a promoter, in a manner which facilitates expression of the coding sequence.

In addition to a promoter, it is also contemplated that the polynucleotide of the disclosure may be operable linked to other regulatory sequences such as enhancers, and other expression control elements that are art-recognized and which may be selected to direct expression of the polynucleotide.

Suitable expression vectors and promoters which may be employed with the polynucleotide of the disclosure are described herein in the context of producing truncated midkine protein and shall be taken to apply mutatis mutandis to each and every example of the disclosure describing polynucleotides unless specifically stated otherwise.

However, in some examples, a promoter useful in the present disclosure can be tissue-specific or cell-specific. The term “tissue specific” as it applies to a promoter refers to a promoter that is capable of directing selective transcription of a nucleic acid of interest to a specific type of tissue (e.g., liver or muscle) in the relative absence of expression of the same nucleotide sequence of interest in a different type of tissue (e.g., kidney). The term “cell-specific” as applied to a promoter refers to a promoter which is capable of directing selective transcription of a nucleic acid of interest in a specific type of cell in the relative absence of expression of the same nucleotide sequence of interest in a different type of cell within the same tissue.

The polynucleotide of the disclosure further comprises one or more multiple cloning sites and/or unique restriction sites that are located strategically, such that the promoter, the sequence encoding the truncated midkine protein and/or other regulator elements are easily removed or replaced. A construct comprising these element can be assembled from smaller oligonucleotide components using strategically located restriction sites and/or complementary sticky ends.

### Production of Polynucleotides

Suitable methods for the production of a polynucleotide of the present disclosure will be apparent to the skilled person and/or described herein.

For example, generation of the DNA sequence and/or a construct comprising same, can be accomplished using any suitable genetic engineering techniques known in the art, including without limitation, the standard techniques of PCR, oligonucleotide synthesis, restriction endonuclease digestion, ligation, transformation, plasmid purification, and DNA sequencing. If a construct comprising the polynucleotide of the disclosure is a viral construct, the construct comprises, for example, sequences necessary to package the polynucleotide of the disclosure into viral particles and/or sequences that allow integration of the polynucleotide of the disclosure into the target cell genome. In some examples, each viral construct additionally contains genes that allow for replication and propagation of virus, however such genes will be supplied in trans. Additionally, each viral construct can contain genes or genetic sequences from the genome of any known organism incorporated in native form or modified. For example, a viral construct may comprise sequences useful for replication of the construct in bacteria.

The construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines.

Other genetic elements that may find use in embodiments of the present disclosure relating to genetic constructs comprising the polynucleotide of the disclosure include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the construct, an internal ribosomal entry site (IRES) sequence can be included. In one example, the additional genetic elements are operably linked with and controlled by an independent promoter/enhancer. In addition a suitable origin of replication for propagation of the construct in bacteria may be employed. The sequence of the origin of replication generally is separated from the cargo polynucleotide sequence and other genetic sequences. Such origins of replication are known in the art and include the pUC, ColE1, 2-micron or SV40 origins of replication.

In other examples, an mRNA of the disclosure may be produced using a plasmid DNA. The skilled person will understand that plasmid DNA is relatively stable. In one exemplary method, competent bacterial cells (e.g., Escherichia coli) cells are transformed with a DNA plasmid encoding the mRNA of the present disclosure. Individual bacterial colonies are isolated and the resultant plasmid DNA amplified in E. coli cultures.

The plasmid DNA is then isolated following fermentation. For example, the plasmid DNA is isolated using a commercially available kit (e.g., Maxiprep DNA kit), or other routine methods known to the skilled person. Following isolation, plasmid DNA is linearized by restriction digest (i.e., using a restricting enzyme). Restriction enzymes are removed using methods known in the art, including for example phenol/chloroform extraction and ethanol precipitation.

In another example, an mRNA of the disclosure may be made by in vitro transcription from a linearized DNA template using an RNA polymerase (e.g., T7 RNA polymerase). Following in vitro transcription, the DNA template is removed by DNase digestion. The skilled person will understand that synthetic RNA capping is performed to correct mRNA processing and contribute to stabilization of the mRNA. In one example, the RNA is enzymatically 5′-capped. For example, the 5′ cap is a cap0 structure or a cap1 structure. In one example, the 5′ cap is a cap0 structure, for example, the 5′-cap (i.e., cap) consists of an inverted 7-methylguanosine connected to the rest of the RNA via a 5′-5′ triphosphate bridge. In one example, the 5′ cap is a cap1 structure, for example, the 5′-cap (i.e., cap 1) consists of the cap0 with an additional methylation of the 2′O position of the initiating polynucleotide. The skilled person will also understand that polyadenylation of the mRNA may be performed in an mRNA comprising a polyadenylation sequence.

The mRNA may also be purified. Various methods for purifying mRNA will be apparent to the skilled person. For example, the mRNA is purified using lithium chloride (LiCl) precipitation. In another example, the mRNA is purified using tangential flow filtration (TFF). In one example, the mRNA is purified using an anion exchange chromatography. For example, anion exchange chromatography is performed using an anion exchange resin (e.g. MustangQ® membrane (Pall®)). Following purification, the mRNA is resuspended in e.g., nuclease-free water.

### Testing of Candidate SSOs

The SSOs of the present disclosure can be conveniently and routinely made through the well-known technique of solid phase synthesis. Any other means for such synthesis known in the art may additionally or alternatively be employed. It is well known to use similar techniques to prepare SSOs such as the phosphorothioates and alkylated derivatives. In this regard, the present disclosure is not limited by the method of SSO synthesis.

Methods of SSO purification and analysis will also be known to those skilled in the art. Analysis methods include capillary electrophoresis (CE) and electrospray-mass spectroscopy. Such synthesis and analysis methods can be performed in multi-well plates. The method of the disclosure is not limited by the method of SSO purification.

Once synthesized, candidate SSOs can be tested for their desired activity using standard procedures and techniques known in the art. For example, screening of candidates may involve administering the candidate SSOs to cells expressing midkine in vitro (e.g., via transfection) and determining the effect of the candidate SSO on expression of midkine in those cells at the mRNA and/or protein level. Detection and quantification of full length and truncated forms of midkine protein can then be performed using standard molecular techniques, such as protein gel electrophoresis and Western blotting. Similarly, detection and quantification of full length and truncated forms of midkine mRNA can then be undertaken using mRNA reverse transcription PCR (RT-PCR) followed by agarose gel electrophoresis, as known in the art. In another example, candidate SSOs may be administered to an animal (e.g., an animal model of midkine-related disease or condition which is known to express midkine), and the animal can be screened for the amount and species midkine mRNA and/or midkine protein expressed. Functional assay may also be performed to assess the effect of candidate SSOs on midkine function in animals to which they are administered. In another example, a candidate SSO may simply be tested for its ability to hybridize to a target polynucleotide (such as mRNA).

### Compositions

The present disclosure also provides a composition comprising the truncated human midkine proteins, the polynucleotides encoding the truncated human midkine proteins and/or the SSOs as described herein for administration. For example, the composition may comprise one or more truncated human midkine proteins described herein. For example, the composition may comprise one or more polynucleotides encoding the truncated human midkine protein described herein. For example, the composition may comprise one or more SSOs targeting human midkine as described herein

Compositions of the disclosure may be admixed, encapsulated, conjugated (such as fused) or otherwise associated with other molecules, molecule structures or mixtures of compounds, resulting in, for example, exosomes, liposomes, Lipid nanoparticles (LNPs), receptor-targeted molecules, oral, rectal, topical, inhalable, injectable or other formulations, for assisting in uptake, distribution and/or absorption (Paunovska K et al (2022) Nature Reviews Genetics 4:1-16). Representative United States patents that teach the preparation of such uptake, distribution and/or absorption-assisting formulations include, but are not limited to, U.S. Pat. Nos. 5,108,921, 5,354,844, 5,416,016, 5,459,127, 5,521,291, 5,543,158, 5,547,932, 5,583,020, 5,591,721, 4,426,330, 4,534,899, 5,013,556, 5,108,921, 5,213,804, 5,227,170, 5,264,221, 5,356,633, 5,395,619, 5,416,016, 5,417,978, 5,462,854, 5,469,854, 5,512,295, 5,527,528, 5,534,259, 5,543,152, 5,556,948, 5,580,575, and 5,595,756.

As used herein, the term “lipid nanoparticle” or “LNP” shall be understood to refer to lipid-based particles having at least one dimension on the order of nanometers (e.g., 1-1,000 nm). LNPs may comprise an ionizable cationic compound, a neutral lipid, charged lipid, sterol and PEGylated lipid. In some examples, the lipid nanoparticle or LNP may be selected from liposomes or vesicles, where an aqueous volume is encapsulated by amphipathic lipid bilayers (e.g., single; unilamellar or 10 multiple; multilamellar), micelle-like lipid nanoparticles having a non-aqueous core and solid lipid nanoparticles. In some examples, the lipid nanoparticle or LNP may have a structure that includes a single monolayer or bilayer of lipids that encapsulates a solid phase. In other examples, the lipid nanoparticle or LNP does not have an aqueous phase or other liquid phase in its interior.

LNPs may comprise bilayer stabilizing component (BSC) such as an ATTA-lipid or a PEG-lipid, such as PEG coupled to dialkyloxypropyls (PEG-DAA) as described in, e.g., WO 05/026372, PEG coupled to diacylglycerol (PEG-DAG) as described in, e.g., U.S. Patent Publication Nos. 20030077829 and 2005008689, PEG coupled to dimyristoylglecerol (PEG-DMG) as described in, e.g., Abrams et. al., Molecular Therapy 2010, 18(1), 171, PEG coupled to phosphatidylethanolamine (PE) (PEG-PE), or PEG conjugated to 1,2-Di-O-hexadecyl-sn-glyceride (PEG-DSG), or a mixture thereof (see, U.S. Pat. No. 5,885,613). For example, the BSC may be a conjugated lipid that inhibits aggregation of the lipid nanoparticle.

For example, the LNP may comprise a neutral lipid, e.g., a phospholipid or an analog or derivative thereof, a structural lipid, e.g., selected from the group consisting of cholesterol, fecosterol, sitosterol, ergosterol, campesterol, stigmasterol, brassicasterol, tomatidine, ursolic acid, alpha-tocopherol, and mixtures thereof, or a PEG lipid, e.g., selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

Truncated human midkine proteins, polynucleotides encoding the truncated human midkine protein or SSOs of the disclosure may be conjugated to one or more moieties or groups which enhance the activity, cellular distribution or cellular uptake of the oligonucleotide or protein. These moieties or groups may be covalently bound to functional groups such as primary or secondary hydroxyl groups. Exemplary moieties or groups include intercalators, reporter molecules, polyamines, polyamides, polyethylene glycols, polyethers, peptides, N-acetylgalactosamine, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins and dyes. SSOs of the disclosure may formulated with one or more pharmaceutically acceptable carriers, diluents or excipients to assist with administration. Alternatively, the truncated human midkine proteins, polynucleotides encoding the truncated human midkine protein or SSOs of the disclosure can be lyophilized for storage and reconstituted in a suitable carrier prior to use according to art-known lyophilization and reconstitution techniques.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

In general terms, by “carrier, diluent or excipient” is meant a solid or liquid filler, binder, diluent, encapsulating substance, emulsifier, wetting agent, solvent, suspending agent, coating or lubricant that may be safely administered to any mammal, e.g., a human. Depending upon the particular route of administration, a variety of acceptable carriers, diluents or excipients, known in the art may be used, as for example described in Remington's Pharmaceutical Sciences (Mack Publishing Co. N.J. USA, 1991). The pharmaceutically acceptable carrier may be solid or liquid. Useful examples of pharmaceutically acceptable carriers include, but are not limited to, diluents, solvents, surfactants, excipients, suspending agents, buffering agents, lubricating agents, adjuvants, vehicles, emulsifiers, absorbants, dispersion media, coatings, stabilizers, protective colloids, adhesives, thickeners, thixotropic agents, penetration agents, sequestering agents, isotonic and absorption delaying agents that do not affect the activity of the active agents of the disclosure.

In one example, the pharmaceutical carrier is water for injection (WFI) and the pharmaceutical composition is adjusted to pH 7.4, 7.2-7.6. In one example, the salt is a sodium or potassium salt.

The truncated human midkine proteins, polynucleotides encoding the truncated human midkine protein or SSOs may contain chiral (asymmetric) centres or the molecule as a whole may be chiral. The individual stereoisomers (enantiomers and diastereoisomers) and mixtures of these are within the scope of the present disclosure.

Compositions of the disclosure may be formulated as pharmaceutically acceptable salts, esters, or salts of the esters, or any other compounds which, upon administration are capable of providing (directly or indirectly) the biologically active metabolite. The term “pharmaceutically acceptable salts” as used herein refers to physiologically and pharmaceutically acceptable salts of the composition that retain the desired biological activities of the parent compounds and do not impart undesired toxicological effects upon administration. Examples of pharmaceutically acceptable salts and their uses are further described in U.S. Pat. No. 6,287,860.

In one example, a truncated human midkine protein, polynucleotide encoding the truncated human midkine protein or SSO of the disclosure can be complexed with a complexing agent to increase its cellular uptake. An example of a complexing agent includes cationic lipids. Cationic lipids can be used to deliver the composition to cells.

The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl−, Br−, I−, F−, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 3000), DOPE, Cytofectin (Gilead Sciences, Foster City, Calif), and Eufectins (JBL, San Luis Obispo, Calif). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3.beta.-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Oligonucleotides and proteins can also be complexed with, e.g., poly (L-lysine) or avidin and lipids may, or may not, be included in this mixture, e.g., steryl-poly (L-lysine).

Cationic lipids have been used in the art to deliver oligonucleotides and proteins to cells (see, e.g., U.S. Pat. Nos. 5,855,910; 5,851,548; 5,830,430; 5,780,053; 5,767,099; U.S. Ser. No. 10/583,201; U.S. Ser. No. 10/912,833; EP15797506.1; Lewis et al., 1996; Hope et al., 1998). Other lipid compositions which can be used to facilitate uptake of the instant compositions can be used in connection with the methods of the disclosure. In addition to those listed above, other lipid compositions are also known in the art and include, e.g., those taught in U.S. Pat. Nos. 4,235,871; 4,501,728; 4,837,028; 4,737,323.

In one example, lipid compositions can further comprise agents, e.g., viral proteins to enhance lipid-mediated transfections of oligonucleotides and proteins. In another example, N-substituted glycine oligonucleotides (peptoids) can be used to optimize uptake of oligonucleotides and proteins.

In another example, a composition of the disclosure for delivery may comprise a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. In particular examples, a preponderance of neutral amino acids with long neutral side chains are used.

In one example, truncated human midkine proteins, polynucleotides encoding the truncated human midkine protein or SSOs of the disclosure are modified by attaching a peptide sequence that assists with transport of the oligonucleotide or protein into a cell, referred to herein as a “transporting peptide” or “cell penetrating peptide (CPP)”. In one example, a truncated human midkine protein, polynucleotide encoding the truncated human midkine protein or SSO of the disclosure is covalently attached to a transporting peptide or a CPP.

As described herein, the truncated midkine protein, polynucleotide encoding same, or SSO of the disclosure may be comprised and provided within an exosome.

As used herein the term “exosome” refers to a cell-derived small (between 20-300 nm in diameter, more preferably 40-200 nm in diameter) vesicle comprising a membrane that encloses an internal space, and which is generated from said cell by direct plasma membrane budding or by fusion of the late endosome with the plasma membrane. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from the producer cell based on its size, density, biochemical parameters, or a combination thereof.

The truncated human midkine proteins, polynucleotides encoding the truncated human midkine protein of the disclosure can be loaded into an exosome or engineered exosome (see, e.g., Olmeda D et al (2017) Nature 546, 676-680). Exosomes may include truncated proteins or polynucleotides that act on a target (e.g. a target cell) that is contacted with the exosome. After being released from an exosome-producing cell, the exosomes may be delivered to a target cell (i.e., recipient cell) where the exosomes are taken up and the exosome cargo (e.g., truncated human midkine proteins, polynucleotides encoding the truncated human midkine protein of the disclosure) is delivered to the cytoplasm of the target cell.

Alternatively, when such an exosome is delivered to the target cells, the exosome cargo can be delivered to cytosol of the target tissue cells through the fusion of the plasma membrane. The said exosome containing a exosome cargo can be used for the treatment of various diseases in vivo. For example, exosome containing a truncated human midkine protein or polynucleotide encoding the same is prepared, which is then delivered to target cells. That is, the exosome can be used as an agent, which exosome is better acting than a conventional liposome.

### In Vitro Functional Assays

Various in vitro assays are available to assess the ability of a truncated human midkine proteins, polynucleotides encoding the truncated human midkine protein or SSOs of the disclosure to inhibit an interaction between human midkine and a ligand thereof and/or midkine activity and/or treat a midkine-related disease or condition.

For example, a “scratch-wound assay” can be used to assess the ability of a truncated midkine protein of the disclosure to inhibit an interaction between human midkine and a ligand thereof and/or inhibit human midkine activity. Such assays are based on the functional migration of cells in vitro or in vivo. Cell migration can be assessed by any suitable means, for example, in an assay utilizing a 96-well plate, or using other art-recognized methods for assessing cell migration.

In yet another example, a chemotaxis assay can be used to assess the ability of a truncated midkine protein of the disclosure to inhibit an interaction between human midkine and a ligand thereof and/or inhibit human midkine activity. These assays are based on the functional migration of cells in vitro or in vivo induced by a compound (chemoattractant). Chemotaxis can be assessed by any suitable means, for example, in an assay utilizing a 96-well chemotaxis plate, or using other art-recognized methods for assessing chemotaxis.

Generally, chemotaxis assays monitor the directional movement or migration of a suitable cell into or through a barrier (e.g., endothelium, a filter), toward increased levels of a compound, from a first surface of the barrier toward an opposite second surface. Membranes or filters provide convenient barriers, such that the directional movement or migration of a suitable cell into or through a filter, toward increased levels of a compound, from a first surface of the filter toward an opposite second surface of the filter, is monitored. In some assays, the membrane is coated with a substance to facilitate adhesion, such as ICAM-1, fibronectin or collagen.

For example, one can detect or measure inhibition of the migration of cells in a suitable container (a containing means), from a first chamber into or through a microporous membrane into a second chamber which contains a chemoattractant e.g., midkine protein, and a truncated midkine protein to be tested, and which is divided from the first chamber by the membrane. A suitable membrane, having a suitable pore size for monitoring specific migration in response to compound, including, for example, nitrocellulose, polycarbonate, is selected. For example, pore sizes of about 3-8 microns, and preferably about 5-8 microns can be used. Pore size can be uniform on a filter or within a range of suitable pore sizes.

To assess migration and inhibition of migration, the distance of migration into the filter, the number of cells crossing the filter that remain adherent to the second surface of the filter, and/or the number of cells that accumulate in the second chamber can be determined using standard techniques (e.g., microscopy and flow cytometry). In one embodiment, the cells are labelled with a detectable label (e.g., radioisotope, fluorescent label, antigen or epitope label), and migration can be assessed in the presence and absence of a truncated midkine protein by determining the presence of the label adherent to the membrane and/or present in the second chamber using an appropriate method (e.g., by detecting radioactivity, fluorescence, immunoassay). The extent of migration induced or inhibited can be determined relative to a suitable control (e.g., compared to background migration determined in the absence of the truncated midkine protein, compared to the extent of migration induced by a second compound (i.e., a standard), compared with migration of untransfected cells induced by the truncated midkine protein).

In one embodiment, a population of cells to which midkine protein binds or which is capable if migrating to midkine protein e.g., a population of UMR106 cells, is placed in a chamber of a cell culture device that is in liquid communication with another chamber comprising midkine protein (chemoattractant). The two chambers are separated by a suitable membrane, e.g., a membrane that mimics the extracellular matrix found in a subject. The amount of cell migration from one chamber to the other through the membrane is assessed in the presence or absence of truncated midkine proteins. A truncated midkine protein that prevents or reduces the amount of midkine-mediated cell migration compared to a control sample (containing no truncated midkine protein) is considered to have midkine inhibitory activity.

An exemplary assay for assessing the ability of a truncated midkine protein described herein to bind midkine protein is a cell migration assay e.g., as described Example 3 herein and Martinotti S and Ranzato E 2020, Scratch wound healing assay. Methods Mol Biol 2109:225, incorporated herein by reference.

In another example, a cell viability assay can be used to assess the ability of a truncated midkine protein of the disclosure on cell proliferation. These assays are based on the functional direct cytotoxic effects or cell death in vitro or in vivo induced by the truncated midkine protein. Cell viability can be assessed by any suitable means, for example, in an assay utilizing a multi-well plate, or using other art recognised methods for assessing cell viability (see for example, Riss et al. 2013, Assay Guidance Manual, Cell Viability Assays).

As will be apparent to the skilled artisan, methods of screening may involve detecting levels of cell death, cell proliferation and/or cell survival. Such methods are known in the art.

### In Vivo Functional Assays

In another example, the efficacy of truncated human midkine of the disclosure to inhibit human midkine activity and/or to treat a disease or condition is assessed using an in vivo assay.

For example, a truncated human midkine of the disclosure may be administered to a non-human mammal (e.g., murine) model of cancer. A truncated human midkine that reduces or alleviates at least one symptom associated with the cancer e.g., tumor size or volume, metastasis e in the mammalian subject relative to the cancer or symptom thereof in the subject prior to administration and/or in a control mammal to which the truncated human midkine has not been administered, is considered suitable for treating the disease or condition.

### Dosages and Regimens

For the prevention or treatment of a disease or condition or relapse thereof, the appropriate dosage of an active agent (e.g., truncated human midkine proteins, polynucleotides encoding the truncated human midkine protein or SSOs of the disclosure), will depend on the type of disease to be treated, the severity and course of the disease, whether the active agent is administered for preventive or therapeutic purposes, previous therapy, the patient's clinical history and response to the active agent, and the discretion of the attending physician. Typically, a therapeutically effective amount of the composition will be administered. The particular dosage regimen, i.e., dose, timing, and repetition, will depend on the particular individual and that individual's medical history as assessed by a physician. Typically, a clinician will administer an active agent until a dosage is reached that achieves the desired result.

As used herein, the terms “treating”, “treat” or “treatment” include administering a therapeutically effective amount of a compound(s) described herein sufficient to reduce or eliminate at least one symptom of a disease, disorder or condition.

As used herein, the terms “preventing”, “prevent” or “prevention” include administering a therapeutically effective amount of a compound(s) described herein sufficient to stop or hinder the development of at least one symptom of a disease, disorder or condition.

The terms “therapeutically effective amount” and “effective amount” describe a quantity of a specified agent, such as an oligonucleotide or protein of the disclosure, sufficient to achieve a desired effect in a subject or cell being treated or contacted with that agent. For example, this can be the amount of a composition comprising one or more agents that inhibit the activity of one or more nucleic acid sensors described herein, necessary to reduce, alleviate and/or prevent a disease, disorder or condition. In some examples, a “therapeutically effective amount” is sufficient to reduce or eliminate a symptom of a disease, disorder or condition. In another example, a “therapeutically effective amount” or “effective amount” is an amount sufficient to achieve a desired biological effect, for example, an amount that is effective to decrease or prevent a senescence-associated disease, disorder or condition or inhibit or prevent senescence in a cell.

Ideally, a therapeutically effective amount of an agent is an amount sufficient to induce the desired result without causing a substantial cytotoxic effect in the subject. The effective amount of an agent useful for reducing, alleviating and/or preventing a disease, disorder or condition will be dependent on the subject being treated, the type and severity of any associated symptoms and the manner of administration of the therapeutic composition.

Generally, the dosage will vary with the age, condition, sex and extent of the disease in the patient and can be determined by one of skill in the art. The dosage can be adjusted by the individual physician in the event of any complication. For in vivo administration of the composition described herein, normal dosage amounts may vary from about 10 ng/kg up to about 100 mg/kg of an individual's body weight or more per day. Exemplary dosages and ranges thereof are described herein. For repeated administrations over several days or longer, depending on the severity of the disease or disorder to be treated, the treatment can be sustained until a desired suppression of symptoms is achieved.

Dosages for a particular compositions may be determined empirically in mammals who have been given one or more administrations of the respective composition. To assess efficacy of a composition of the disclosure, a clinical symptom of a composition or condition being treated e.g., cancer, can be monitored. For example, efficacy of an SSO of the disclosure in treatment of cancer may be assessed based on tumour size and/or using diagnostic, prognostic or predictive biomarkers of cancer.

Administration of a composition according to the methods of the present disclosure can be continuous or intermittent, depending, for example, on the recipient's physiological condition, whether the purpose of the administration is therapeutic or prophylactic, and other factors known to skilled practitioners. The administration of an composition may be essentially continuous over a preselected period of time or may be in a series of spaced doses.

A variety of routes of administration are possible including, but not necessarily limited to, oral, dietary, topical, parenteral (e.g., intravenous, intraarterial, intramuscular, subcutaneous injection), inhalation (e.g., intrabronchial, intraocular, intranasal or oral inhalation, intranasal drops), depending on the disease or condition to be treated. Other suitable methods of administration can also include rechargeable or biodegradable devices and slow-release biologic or synthetic polymeric devices.

### Uses

Compositions of the present disclosure have the ability to inhibit midkine function and can therefore be used as therapeutic and preventative drugs for midkine-related diseases and disorders.

In one example, the present disclosure provides a method for inhibiting an interaction between human midkine and a ligand thereof on the surface of or in a cell, said method comprising exposing the cell to the composition described herein. It will be appreciated by the skilled person that the method may be performed in vitro, ex vivo or in vivo.

The cell may be any known in the art that expresses midkine. By way of example, the cell can be an immune cell, such as T cells, B cells, natural killer cells, neutrophils, eosinophils, mast cells, basophils, monocytes, macrophages and dendritic cells; endothelial cells; or neurones. In oncology the cells can be malignant tumour cells or stromal cells, such as fibroblasts or endothelial cells.

In another example, the disclosure provides a method for inhibiting human midkine activity in a cell, said method comprising exposing the cell to the composition described herein.

As used herein, the term “inhibit” shall be taken to mean hinder, reduce, restrain or prevent midkine activity in a cell relative to midkine activity in a cell in which the truncated human midkine protein or polynucleotide encoding the truncated human midkine protein of the present disclosure is present or the SSO of the disclosure is absent.

Midkine activity may be inhibited in any measurable amount. Inhibition of midkine activity may be complete or may be partial. Thus, the methods disclosed herein may comprise at least partial inhibition of midkine activity. For example, the activity of midkine may be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% following contacting the cell with an effective amount of the composition (e.g., relative to the same measurement of activity before contact with the composition).

In another example, the disclosure provides a method for treating or preventing a midkine-related disease or disorder in a subject in need thereof, said method comprising administering to the subject the composition described herein.

In yet another example, the disclosure provides for use of the composition described herein, in the preparation of a medicament for treatment or prevention of a midkine-related disease or disorder,

The term “midkine-related disease” refers to a disease involving midkine functions. Examples of such diseases include: diseases attributed to cell growth or angiogenesis, such as cancers (esophageal cancer, thyroid cancer, urinary bladder cancer, colon cancer, stomach cancer, pancreatic cancer, thoracic cancer, liver cancer, lung cancer, breast cancer, neuroblastoma, glioblastoma, uterine cancer, ovarian cancer, prostatic cancer, and Wilms tumor) and endometriosis; inflammatory diseases or diseases attributed to recruitment of inflammatory cells, suppression of regulatory T cell function, activation of neutrophils, dysfunctional orientation of macrophages or T cells such as inflammatory diseases of the kidney, acute renal failure, chronic kidney diseases, osteoporosis, sepsis, arthritis, autoimmune disease (organ-specific autoimmune disease, etc.), rheumatic arthritis (rheumatoid arthritis (RA) or osteoarthritis (OA)), multiple sclerosis (relapsing-remitting multiple sclerosis, etc.), inflammatory bowel disease (Crohn disease, etc.), systemic lupus erythematosus (SLE), progressive systematic sclerosis (PSS), Sjogren's syndrome, polymyositis (PM), dermatomyositis (DM), polyarteritis nodosa (PN), thyroid disease (Graves disease, etc.), Guillain-Barre syndrome, primary biliary cirrhosis (PBC), idiopathic thrombocytopenic purpura, autoimmune hemolytic anemia, experimental autoimmune myasthenia gravis (EAMG), amyotrophic lateral sclerosis (ALS), type I diabetes mellitus, transplant rejection, postoperative adhesion, endometriosis, psoriasis, lupus, allergy, asthma, acute respiratory distress syndrome; ventilation induced lung injury; and occlusive vascular diseases or diseases attributed to vascular intimal thickening, such as post-revascularization restenosis, coronary occlusive disease, cerebrovascular occlusive disease, renovascular occlusive disease, peripheral occlusive disease, arteriosclerosis, and cerebral infarction. In one example, the midkine-related disease or disorder is selected from an autoimmune disease, cancer, or an inflammatory disease.

In particular examples, the disease to be treated or prevented using a composition of the disclosure demonstrates increased, excessive or abnormal midkine expression, accumulation, activity and/or signalling. Such diseases are described herein, albeit without limitation thereto. In one example, the compositions of the disclosure may be used in methods of preventing or inhibiting inflammation associated with administration of a therapeutic SSO, such as those known in the art, to a subject. In particular, the SSOs described herein may be used in the prevention or inhibition of inflammation mediated by one or more nucleic acid sensors (e.g., TLR3, TLR7, TLR8, TLR9, cGAS, RIG-I) during or following administration of the therapeutic SSO. It is envisaged that the inflammation may involve or include any cells, tissues or organs of the body. In particular examples, the inflammation is or comprises hepatic inflammation. To this end, the therapeutic SSO may be conjugated to N-acetylgalactosamine (GalNAc), which enhances asialoglycoprotein receptor (ASGR)-mediated uptake into liver hepatocytes (Nair et al., 2014), and thereby enabling their specific targeting to the liver.

In particular examples, therapeutically effective amounts of the therapeutic SSO and the composition of the disclosure may be administered simultaneously, concurrently, sequentially, successively, alternately or separately in any particular combination and/or order.

In accordance with the above-mentioned uses, the compositions of the disclosure may be administered to an animal. For example, the animal (or subject) to which the composition is administered may be a mammal, avian, chordate, amphibian or reptile. In one example, the animal is a mammal. Exemplary mammalian subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one example, the mammal is a human.

### Combination Therapies

The compositions of the present disclosure can also be administered as part of a combinatorial therapy with other agents useful for treating a disease or condition, e.g., cancer, either as combined or additional treatment steps or as additional components of a therapeutic formulation. Such other therapies/agents will be well-known to those skilled in the art.

For example, the other compound is an anti-inflammatory or immunomodulatory drug. Alternatively, or additionally, the other compound is an immunosuppressant. Alternatively, or additionally, the other compound is a chemotherapeutic agent, such as carboplatin.

So that preferred examples of the present disclosure may be fully understood and put into practical effect, reference is made to the following non-limiting examples.

TABLE 1
Midkine primer sets

| | | SEQ ID |
|---|---|---|
| Region ID | Sequence (5′-3′) | NO: |
| hMDK ExIF | AGACCGGTTCTGGAGACAAA | 1 |
| hMDK Ex5R (o) | GCTCTGGGACTCACATTGCT | 2 |
| hMDK Ex5R (i) | TCCCAGAATCCCTTGTCCCT | 3 |
| hMDK Ex2F (o) | AGGCTTCCTCCTCCTCACC | 4 |
| hMDK Ex2F (i) | CGCGGTCGCCAAAAAGAAAG | 5 |

TABLE 2
Target regions within midkine protein

| Region | | SEQ ID |
|---|---|---|
| ID | Region sequence (5′-3′) | NO: |
| MDK | AGACCGGUUCUGGAGACAAAAGGGGCCGCGGCGGCCGGAGCGGGA | 6 |
| mRNA | CGGGCCCGGCGCGGGAGGGAGCGAAGCAGCGCGGGCAGCGAGCGA | |
| full length | GAUGCAGCACCGAGGCUUCCUCCUCCUCACCCUCCUCGCCCUGCUG | |
| | GCGCUCACCUCCGCGGUCGCCAAAAAGAAAGAUAAGGUGAAGAAG | |
| | GGCGGCCCGGGGAGCGAGUGCGCUGAGUGGGCCUGGGGGCCCUGC | |
| | ACCCCCAGCAGCAAGGAUUGCGGCGUGGGUUUCCGCGAGGGCACC | |
| | UGCGGGGCCCAGACCCAGCGCAUCCGGUGCAGGGUGCCCUGCAAC | |
| | UGGAAGAAGGAGUUUGGAGCCGACUGCAAGUACAAGUUUGAGAAC | |
| | UGGGGUGCGUGUGAUGGGGGCACAGGCACCAAAGUCCGCCAAGGC | |
| | ACCCUGAAGAAGGCGCGCUACAAUGCUCAGUGCCAGGAGACCAUC | |
| | CGCGUCACCAAGCCCUGCACCCCCAAGACCAAAGCAAAGGCCAAAG | |
| | CCAAGAAAGGGAAGGGAAAGGACUAGACGCCAAGCCUGGAUGCCA | |
| | AGGAGCCCCUGGUGUCACAUGGGGCCUGGCCCACGCCCUCCCUCUC | |
| | CCAGGCCCGAGAUGUGACCCACCAGUGCCUUCUGUCUGCUCGUUA | |
| | GCUUUAAUCAAUCAUGCCCUGCCUUGUCCCUCUCACUCCCCAGCCC | |
| | CACCCCUAAGUGCCCAAAGUGGGGAGGGACAAGGGAUUCUGGGAA | |
| | GCUUGAGCCUCCCCCAAAGCAAUGUGAGUCCCAGAGCCCGCUUUU | |
| | GUUCUUCCCCACAAUUCCAUUACUAAGAAACACAUCAAAUAAACU | |
| | GACUUUUUCCCCCCAAAAAAAAAAAAAAAAAA | |
| MDK Region | GCAGGGTGCCCTGCAACTGGAAGAAGGAGTTTGGAGGTGAGGCGGG | 7 |
| 1 | GCGCAGTCAG | |
| MDK Region | GCAGGGTGCCCTGCAACTGGAAGAAGGAGTTTGGA | 8 |
| 2 | | |
| MDK Region | GCAGGGTGCCCTGCAACTGGAAGAAGGAGT | 9 |
| 3 | | |
| MDK Region | CAACTGGAAGAAGGAGTTTGGA | 10 |
| 4 | | |
| MDK Region | CAACTGGAAGAAGGAGTTTGGAGGTGAGGCGGG | 11 |
| 5 | | |
| MDK Region | ACTGGAAGAAGGAGTTTGGAGGTGAGGCGGGGCGCAGTCAG | 12 |
| 6 | | |
| MDK Region | ACTGGAAGAAGGAGTTTGGAGGTGAGGCGGG | 13 |
| 7 | | |
| MDK Region | GAGTTTGGAGGTGAGGCGGGGCGCAGTCAG | 14 |
| 8 | | |
| MDK Region | GGCACCCTGAAGAAGGCGCGCTACAATGCTCAGTGCCAGGAGACCA | 15 |
| 9 | TCCGCGTCACCAAGCCCTGCACCCCCAAGACCAAAGCAAAGGCCAA | |
| | AGGTCAGCGAAAGGAGAAGGGG | |
| MDK Region | GGCACCCTGAAGAAGGCGCGCTACAATGCTCAGTGCCAGGAGACCA | 16 |
| 10 | TCCGCGTCACCAAGCCCTGCA | |
| MDK Region | GGCACCCTGAAGAAGGCGCGCTACAATGCTCAGTG | 17 |
| 11 | | |
| MDK Region | GTGCCAGGAGACCATCCGCGTCACCAAGCCCTGCA | 18 |
| 12 | | |
| MDK Region | AAGCAAAGGCCAAAGGTCAGCGAAAGGAGAAGGGG | 19 |
| 13 | | |
| MDK Region | CGCGCGTTGTAGATAAGGTGAAGAA | 20 |
| 14 | | |
| MDK Region | GTAGATAAGGTGAAGAAGGGCGGCC | 21 |
| 15 | | |
| MDK Region | TTTCCGCGAGGGCACCTGCGGGGCC | 22 |
| 16 | | |
| MDK Region | CTGCAACTGGAAGAAGGAGTTTGGA | 23 |
| 17 | | |
| MDK Region | CAACTGGAAGAAGGAGTTTG | 24 |
| 18 | | |
| MDK Region | AGAAGGAGTTTGGAGGTGAGGCGGG | 25 |
| 19 | | |
| MDK Region | CTCTCTCGCCAGCCGACTGCAAGTA | 26 |
| 20 | | |
| MDK Region | CCAGCCGACTGCAAGTACAAGTTTG | 27 |
| 21 | | |
| MDK Region | CCTGAAGAAGGCGCGCTACAATGCT | 28 |
| 22 | | |
| MDK Region | AGGAGACCATCCGCGTCACCAAGCC | 29 |
| 23 | | |
| MDK Region | AAGGCCAAAGGTCAGCGAAAGGAGA | 30 |
| 24 | | |
| MDK Region | GCAGGGTGCCCTGCAACTGGAAGAA | 31 |
| 25 | | |
| MDK Region | GTGCCCTGCAACTGGAAGAAGGAGT | 32 |
| 26 | | |
| MDK Region | ACTGGAAGAAGGAGTTTGGAGGTGA | 33 |
| 27 | | |
| MDK Region | GAGTTTGGAGGTGAGGCGGGGCGCA | 34 |
| 28 | | |
| MDK Region | GAGTTTGGAGGTGAGGCGGGGCGCA | 35 |
| 29 | | |
| MDK Region | GGCACCCTGAAGAAGGCGCGCTACA | 36 |
| 30 | | |
| MDK Region | AGAAGGCGCGCTACAATGCTCAGTG | 37 |
| 31 | | |
| MDK Region | GTGCCAGGAGACCATCCGCGTCACC | 38 |
| 32 | | |
| MDK Region | ACCATCCGCGTCACCAAGCCCTGCA | 39 |
| 33 | | |
| MDK Region | AAGCAAAGGCCAAAGGTCAGCGAAA | 40 |
| 34 | | |
| MDK Region | CAAAGGTCAGCGAAAGGAGAAGGGG | 41 |
| 35 | | |
| MDK Region | AGTGCCAGGAGACCATCCGCGTCAC | 42 |
| 36 | | |

TABLE 3
SSOs

| | | SEQ ID |
|---|---|---|
| Region ID | Sequence (5′-3′) | NO: |
| MDK H3A(−12+13) | UUCUUCACCUUAUCUACAACGCGCG | 43 |
| MDK H3A(−04+21) | GGCCGCCCUUCUUCACCUUAUCUAC | 44 |
| MDK H3A(+89+113) | GGCCCCGCAGGUGCCCUCGCGGAAA | 45 |
| MDK H3A(+143+167) | UCCAAACUCCUUCUUCCAGUUGCAG | 46 |
| MDK H3A(+146+165) | AAACUCCUUCUUCCAGUUGC | 47 |
| MDK H3D(+15−10) | CCCGCCUCACCUCCAAACUCCUUCU | 48 |
| MDK H4A(−12+13) | UACUUGCAGUCGGCUGGCGAGAGAG | 49 |
| MDK H4A(−04+21) | CAAACUUGUACUUGCAGUCGGCUGG | 50 |
| MDK H4A(+74+98) | AGCAUUGUAGCGCGCCUUCUUCAGG | 51 |
| MDK H4A(+106+130) | GGCUUGGUGACGCGGAUGGUCUCCU | 52 |
| MDK H4D(+10−15) | UCUCCUUUCGCUGACCUUUGGCCUU | 53 |
| MDK H3A(+133+157) | UUCUUCCAGUUGCAGGGCACCCUGC | 54 |
| MDK H3A(+138+162) | ACUCCUUCUUCCAGUUGCAGGGCAC | 55 |
| MDK H3D(+21−04) | UCACCUCCAAACUCCUUCUUCCAGU | 56 |
| MDK H3D(+10−15) | UGCGCCCCGCCUCACCUCCAAACUC | 57 |
| MDK H3D(+05−20) | CUGACUGCGCCCCGCCUCACCUCCA | 58 |
| MDK H4A(+69+93) | UGUAGCGCGCCUUCUUCAGGGUGCC | 59 |
| MDK H4A(+79+103) | CACUGAGCAUUGUAGCGCGCCUUCU | 60 |
| MDK H4A(+101+125) | GGUGACGCGGAUGGUCUCCUGGCAC | 61 |
| MDK H4A(+111+135) | UGCAGGGCUUGGUGACGCGGAUGGU | 62 |
| MDK H4D(+15−10) | UUUCGCUGACCUUUGGCCUUUGCUU | 63 |
| MDK H4D(+05−20) | CCCCUUCUCCUUUCGCUGACCUUUG | 64 |
| MDK H4A(+100+124) | GTGACGCGGATGGTCTCCTGGCACT | 65 |

TABLE 4
Midkine exon sequences and truncated midkine proteins

| | | SEQ ID |
|---|---|---|
| Sequence ID | Sequence (5′-3′) | NO: |
| RNA sequence of Exon 1 | AGACCGGUUCUGGAGACAAAAGGGGCCGCGGCGGCCG | 66 |
| -5′UTR | GAGCGGGACGGGCCCGGCGCGGGAGGGAGCGAAGCAG | |
| | CGCGGGCAGCGAGCGA | |
| RNA sequence of Exon 2 | GAUGCAGCACCGAGGCUUCCUCCUCCUCACCCUCCUCG | 67 |
| | CCCUGCUGGCGCUCACCUCCGCGGUCGCCAAAAAGAA | |
| | AG | |
| Amino acid sequence of | MQHRGFLLLTLLALLALTSAVAKKK | 68 |
| Exon 2 | | |
| RNA sequence of Exon 3 | AUAAGGUGAAGAAGGGCGGCCCGGGGAGCGAGUGCGC | 69 |
| | UGAGUGGGCCUGGGGGCCCUGCACCCCCAGCAGCAAG | |
| | GAUUGCGGCGUGGGUUUCCGCGAGGGCACCUGCGGGG | |
| | CCCAGACCCAGCGCAUCCGGUGCAGGGUGCCCUGCAA | |
| | CUGGAAGAAGGAGUUUGGAG | |
| Amino acid sequence of | KVKKGGPGSECAEWAWGPCTPSSKDCGVGFREGTCGAQT | 70 |
| Exon 3 | QRIRCRVPCNWKKEFG | |
| DNA sequence of Exon 4 | CCGACTGCAAGTACAAGTTTGAGAACTGGGGTGCGTGT | 71 |
| | GATGGGGGCACAGGCACCAAAGTCCGCCAAGGCACCCT | |
| | GAAGAAGGCGCGCTACAATGCTCAGTGCCAGGAGACCA | |
| | TCCGCGTCACCAAGCCCTGCACCCCCAAGACCAAAGCA | |
| | AAGGCCAAAG | |
| RNA sequence of Exon 4 | CCGACUGCAAGUACAAGUUUGAGAACUGGGGUGCGUG | 72 |
| | UGAUGGGGGCACAGGCACCAAAGUCCGCCAAGGCACC | |
| | CUGAAGAAGGCGCGCUACAAUGCUCAGUGCCAGGAGA | |
| | CCAUCCGCGUCACCAAGCCCUGCACCCCCAAGACCAAA | |
| | GCAAAGGCCAAAG | |
| Amino acid sequence of | DCKYKFENWGACDGGTGTKVRQGTLKKARYNAQCQETI | 73 |
| Exon 4 | RVTKPCTPKTKAKAK | |
| RNA Sequence of Exon 5 | ACCAAGCCCUGCACCCCCAAGACCAAAGCAAAGGCCA | 74 |
| -C-terminal and 3′UTR | AAGCCAAGAAAGGGAAGGGAAAGGACUAGACGCCAAG | |
| | CCUGGAUGCCAAGGAGCCCCUGGUGUCACAUGGGGCC | |
| | UGGCCCACGCCCUCCCUCUCCCAGGCCCGAGAUGUGAC | |
| | CCACCAGUGCCUUCUGUCUGCUCGUUAGCUUUAAUCA | |
| | AUCAUGCCCUGCCUUGUCCCUCUCACUCCCCAGCCCCA | |
| | CCCCUAAGUGCCCAAAGUGGGGAGGGACAAGGGAUUC | |
| | UGGGAAGCUUGAGCCUCCCCCAAAGCAAUGUGAGUCC | |
| | CAGAGCCCGCUUUUGUUCUUCCCCACAAUUCCAUUAC | |
| | UAAGAAACACAUCAAAUAAACUGACUUUUUCCCCCCA | |
| | AAAAAA | |
| Midkine protein | MQHRGFLLLTLLALLALTSAVAKKKDKVKKGGPGSECAE | 75 |
| full length | WAWGPCTPSSKDCGVGFREGTCGAQTQRIRCRVPCNWKK | |
| | EFGADCKYKFENWGACDGGTGTKVRQGTLKKARYNAQC | |
| | QETIRVTKPCTPKTKAKAKAKKGKGKD | |
| Exon 4 truncated MDK | AGACCGGTTCTGGAGACAAAAGGGGCCGCGGCGGCCG | 76 |
| DNA | GAGCGGGACGGGCCCGGCGCGGGAGGGAGCGAAGCAG | |
| | CGCGGGCAGCGAGCGAGATGCAGCACCGAGGCTTCCTC | |
| | CTCCTCACCCTCCTCGCCCTGCTGGCGCTCACCTCCGCG | |
| | GTCGCCAAAAAGAAAGATAAGGTGAAGAAGGGCGGCC | |
| | CGGGGAGCGAGTGCGCTGAGTGGGCCTGGGGGCCCTGC | |
| | ACCCCCAGCAGCAAGGATTGCGGCGTGGGTTTCCGCGA | |
| | GGGCACCTGCGGGGCCCAGACCCAGCGCATCCGGTGCA | |
| | GGGTGCCCTGCAACTGGAAGAAGGAGTTTGGAGCCAAG | |
| | AAAGGGAAGGGAAAGGACTAGACGCCAAGCCTGGATG | |
| | CCAAGGAGCCCCTGGTGTCACATGGGGCCTGGCCCACG | |
| | CCCTCCCTCTCCCAGGCCCGAGATGTGACCCACCAGTGC | |
| | CTTCTGTCTGCTCGTTAGCTTTAATCAATCATGCCCTGCC | |
| | TTGTCCCTCTCACTCCCCAGCCCCACCCCTAAGTGCCCA | |
| | AAGTGGGGAGGGACAAGGGATTCTGGGAAGCTTGAGCC | |
| | TCCCCCAAAGCAATGTGAGTCCCAGAGCCCGCTTTTGTT | |
| | CTTCCCCACAATTCCATTACTAAGAAACACATCAAATAA | |
| | ACTGACTTTTTCCCCCCAAAAAAAAAAAAAAAAAA | |
| Exon 4 truncated MDK | AGACCGGUUCUGGAGACAAAAGGGGCCGCGGCGGCCG | 77 |
| mRNA | GAGCGGGACGGGCCCGGCGCGGGAGGGAGCGAAGCAG | |
| | CGCGGGCAGCGAGCGAGAUGCAGCACCGAGGCUUCCU | |
| | CCUCCUCACCCUCCUCGCCCUGCUGGCGCUCACCUCCG | |
| | CGGUCGCCAAAAAGAAAGAUAAGGUGAAGAAGGGCGG | |
| | CCCGGGGAGCGAGUGCGCUGAGUGGGCCUGGGGGCCC | |
| | UGCACCCCCAGCAGCAAGGAUUGCGGCGUGGGUUUCC | |
| | GCGAGGGCACCUGCGGGGCCCAGACCCAGCGCAUCCG | |
| | GUGCAGGGUGCCCUGCAACUGGAAGAAGGAGUUUGGA | |
| | GCCAAGAAAGGGAAGGGAAAGGACUAGACGCCAAGCC | |
| | UGGAUGCCAAGGAGCCCCUGGUGUCACAUGGGGCCUG | |
| | GCCCACGCCCUCCCUCUCCCAGGCCCGAGAUGUGACCC | |
| | ACCAGUGCCUUCUGUCUGCUCGUUAGCUUUAAUCAAU | |
| | CAUGCCCUGCCUUGUCCCUCUCACUCCCCAGCCCCACC | |
| | CCUAAGUGCCCAAAGUGGGGAGGGACAAGGGAUUCUG | |
| | GGAAGCUUGAGCCUCCCCCAAAGCAAUGUGAGUCCCA | |
| | GAGCCCGCUUUUGUUCUUCCCCACAAUUCCAUUACUA | |
| | AGAAACACAUCAAAUAAACUGACUUUUUCCCCCCAAA | |
| | AAAAAAAAAAAAAAA | |
| Exon 4 truncated MDK | MQHRGFLLLTLLALLALTSAVAKKKDKVKKGGPGSECAE | 78 |
| protein | WAWGPCTPSSKDCGVGFREGTCGAQTQRIRCRVPCNWKK | |
| | EFGAKKGKGKD | |
| DNA sequence of 5′ | CCGACTGCAAGTACAAG | 79 |
| fragment of Exon 4 | | |
| DNA sequence of 3′ | AAAGCAAAGGCCAAAG | 80 |
| fragment of Exon 4 | | |
| RNA sequence of 5′ | CCGACUGCAAGUACAAG | 81 |
| fragment of Exon 4 | | |
| RNA sequence of 3′ | AAAGCAAAGGCCAAAG | 82 |
| fragment of Exon 4 | | |
| Amino acid sequence of 5′ | DCKYK | 83 |
| fragment of Exon 4 | | |
| Amino acid sequence of 3′ | KAKAK | 84 |
| fragment of Exon 4 | | |
| Amino acid sequence of | AKKGKGKD | 85 |
| Exon 5 | | |
| RNA sequence of Δ14 | AUGCAGCACCGAGGCUUCCUCCUCCUCACCCUCCUCGC | 86 |
| MDK | CCUGCUGGCGCUCACCUCCGCGGUCGCCAAAAAGAAA | |
| | GAUAAGGUGAAGAAGGGCGGCCCGGGGAGCGAGUGCG | |
| | CUGAGUGGGCCUGGGGGCCCUGCACCCCCAGCAGCAA | |
| | GGAUUGCGGCGUGGGUUUCCGCGAGGGCACCUGCGGG | |
| | GCCCAGACCCAGCGCAUCCGGUGCAGGGUGCCCUGCA | |
| | ACUGGAAGAAGGAGUUUGGAGCCGACUGCAAGUACAA | |
| | GUUUGAGAACUGGGGUGCGUGUGAUGGGGGCACAGGC | |
| | ACCAAAGUCCGCCAAGGCACCCUGAAGAAGGCGCGCU | |
| | ACAAUGCUCAGUGCCAGGAGACCAUCCGCGUCACCAA | |
| | GCCCUGCACCCCCAAGUAG | |
| Amino acid sequence of | MQHRGFLLLTLLALLALTSAVAKKKDKVKKGGPGSECAE | 87 |
| Δ14 MDK | WAWGPCTPSSKDCGVGFREGTCGAQTQRIRCRVPCNWKK | |
| | EFGADCKYKFENWGACDGGTGTKVRQGTLKKARYNAQC | |
| | QETIRVTKPCTPK | |
| RNA sequence of Δ25 | AUGCAGCACCGAGGCUUCCUCCUCCUCACCCUCCUCGC | 88 |
| MDK | CCUGCUGGCGCUCACCUCCGCGGUCGCCAAAAAGAAA | |
| | GAUAAGGUGAAGAAGGGCGGCCCGGGGAGCGAGUGCG | |
| | CUGAGUGGGCCUGGGGGCCCUGCACCCCCAGCAGCAA | |
| | GGAUUGCGGCGUGGGUUUCCGCGAGGGCACCUGCGGG | |
| | GCCCAGACCCAGCGCAUCCGGUGCAGGGUGCCCUGCA | |
| | ACUGGAAGAAGGAGUUUGGAGCCGACUGCAAGUACAA | |
| | GUUUGAGAACUGGGGUGCGUGUGAUGGGGGCACAGGC | |
| | ACCAAAGUCCGCCAAGGCACCCUGAAGAAGGCGCGCU | |
| | ACAAUGCUCAGUGCCAGGAGUAG | |
| Amino acid sequence of | MQHRGFLLLTLLALLALTSAVAKKKDKVKKGGPGSECAE | 89 |
| Δ25 MDK | WAWGPCTPSSKDCGVGFREGTCGAQTQRIRCRVPCNWKK | |
| | EFGADCKYKFENWGACDGGTGTKVRQGTLKKARYNAQC | |
| | QE | |
| RNA sequence of Δ34 | AUGCAGCACCGAGGCUUCCUCCUCCUCACCCUCCUCGC | 90 |
| MDK | CCUGCUGGCGCUCACCUCCGCGGUCGCCAAAAAGAAA | |
| | GAUAAGGUGAAGAAGGGCGGCCCGGGGAGCGAGUGCG | |
| | CUGAGUGGGCCUGGGGGCCCUGCACCCCCAGCAGCAA | |
| | GGAUUGCGGCGUGGGUUUCCGCGAGGGCACCUGCGGG | |
| | GCCCAGACCCAGCGCAUCCGGUGCAGGGUGCCCUGCA | |
| | ACUGGAAGAAGGAGUUUGGAGCCGACUGCAAGUACAA | |
| | GUUUGAGAACUGGGGUGCGUGUGAUGGGGGCACAGGC | |
| | ACCAAAGUCCGCCAAGGCACCCUGAAGAAGUAG | |
| Amino acid sequence of | MQHRGFLLLTLLALLALTSAVAKKKDKVKKGGPGSECAE | 91 |
| Δ34 MDK | WAWGPCTPSSKDCGVGFREGTCGAQTQRIRCRVPCNWKK | |
| | EFGADCKYKFENWGACDGGTGTKVRQGTLKK | |
| RNA sequence of Δ41 | AUGCAGCACCGAGGCUUCCUCCUCCUCACCCUCCUCGC | 92 |
| MDK | CCUGCUGGCGCUCACCUCCGCGGUCGCCAAAAAGAAA | |
| | GAUAAGGUGAAGAAGGGCGGCCCGGGGAGCGAGUGCG | |
| | CUGAGUGGGCCUGGGGGCCCUGCACCCCCAGCAGCAA | |
| | GGAUUGCGGCGUGGGUUUCCGCGAGGGCACCUGCGGG | |
| | GCCCAGACCCAGCGCAUCCGGUGCAGGGUGCCCUGCA | |
| | ACUGGAAGAAGGAGUUUGGAGCCGACUGCAAGUACAA | |
| | GUUUGAGAACUGGGGUGCGUGUGAUGGGGGCACAGGC | |
| | ACCAAAGUCUAG | |
| Amino acid sequence of | MQHRGFLLLTLLALLALTSAVAKKKDKVKKGGPGSEC | 93 |
| Δ41 MDK | AEWAWGPCTPSSKDCGVGFREGTCGAQTQRIRCRVPC | |
| | NWKKEFGADCKYKFENWGACDGGTGTKV | |
| RNA sequence of Δ58 | AUGCAGCACCGAGGCUUCCUCCUCCUCACCCUCCUCGC | 94 |
| MDK | CCUGCUGGCGCUCACCUCCGCGGUCGCCAAAAAGAAA | |
| | GAUAAGGUGAAGAAGGGCGGCCCGGGGAGCGAGUGCG | |
| | CUGAGUGGGCCUGGGGGCCCUGCACCCCCAGCAGCAA | |
| | GGAUUGCGGCGTGGGUUUCCGCGAGGGCACCUGCGGG | |
| | GCCCAGACCCAGCGCAUCCGGUGCAGGGUGCCCUGCA | |
| | ACUGGAAGAAGGAGUUUGGAGCCGACUGCAAGUAG | |
| Amino acid sequence of | MQHRGFLLLTLLALLALTSAVAKKKDKVKKGGPGSEC | 95 |
| Δ58 MDK | AEWAWGPCTPSSKDCGVGFREGTCGAQTQRIRCRVPC | |
| | NWKKEFGADCK | |
| MDK | AUGCAGCACCGAGGCUUCCUCCUCCUCACCCUCCUCGC | 96 |
| mRNA | CCUGCUGGCGCUCACCUCCGCGGUCGCCAAAAAGAAA | |
| full length including the | GAUAAGGUGAAGAAGGGCGGCCCGGGGAGCGAGUGCG | |
| natural start codon and | CUGAGUGGGCCUGGGGGCCCUGCACCCCCAGCAGCAA | |
| peptide leader sequence | GGAUUGCGGCGUGGGUUUCCGCGAGGGCACCUGCGGG | |
| | GCCCAGACCCAGCGCAUCCGGUGCAGGGUGCCCUGCA | |
| | ACUGGAAGAAGGAGUUUGGAGCCGACUGCAAGUACAA | |
| | GUUUGAGAACUGGGGUGCGUGUGAUGGGGGCACAGGC | |
| | ACCAAAGUCCGCCAAGGCACCCUGAAGAAGGCGCGCU | |
| | ACAAUGCUCAGUGCCAGGAGACCAUCCGCGUCACCAA | |
| | GCCCUGCACCCCCAAGACCAAAGCAAAGGCCAAAGCC | |
| | AAGAAAGGGAAGGGAAAGGACUAG | |

### EXAMPLES

### Example 1: Primer Design and Optimization

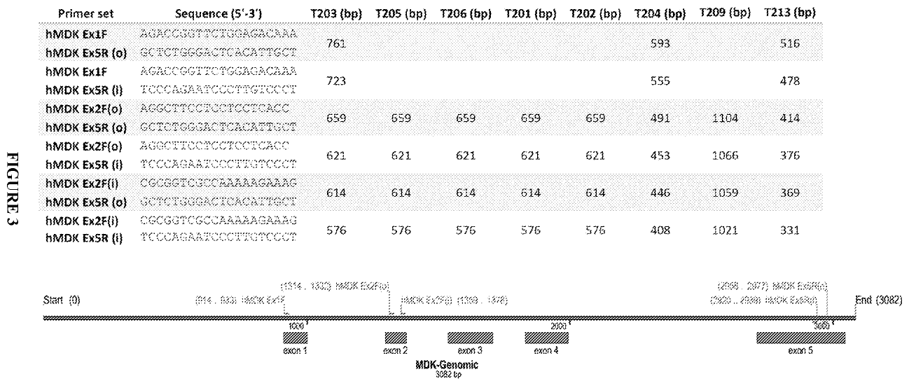

There are nine protein-coding MDK transcripts reported on Ensembl. The MDK-203 transcript (NM_002391.6) was used as the basis of the exon numbering. The majority of transcripts differ only by their 5′ untranslated region; however, transcripts 204, 209, 207 and 213 also have changes within the coding region (see FIG. 2).

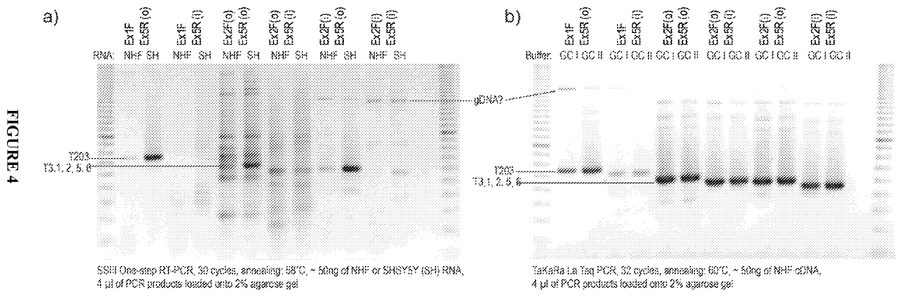

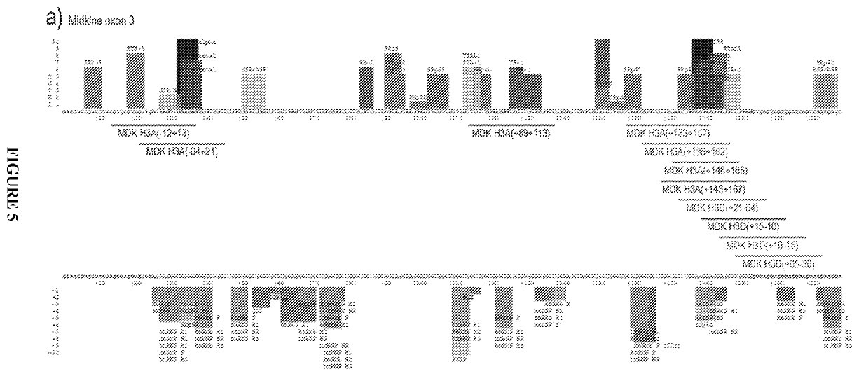

Five primers were designed to amplify the MDK regions of interest. The sequence and expected polymerase chain reaction (PCR) product sizes of the various primer set are outlined in Table 5. The binding location of each primer on the MDK genomic sequence is shown in FIG. 3.

TABLE 5
Midkine primer sets, their sequences and expected product size.

| | | T203 | T205 | T206 | T201 | T202 | T204 | T209 | T213 |
|---|---|---|---|---|---|---|---|---|---|
| Primer set | Sequence (5′-3′) | (bp) | (bp) | (bp) | (bp) | (bp) | (bp) | (bp) | (bp) |
| hMDK Ex1F | AGACCGGTTCTGGAGACAAA | 761 | | | | | 593 | | 516 |
| hMDK Ex5r (o) | GCTCTGGGACTCACATTGCT | | | | | | | | |
| hMDK Ex1F | AGACCGGTTCTGGAGACAAA | 723 | | | | | 555 | | 478 |
| hMDK Ex5R (i) | TCCCAGAATCCCTTGTCCCT | | | | | | | | |
| hMDK Ex2F (o) | AGGCTTCCTCCTCCTCACC | 659 | 659 | 659 | 659 | 659 | 491 | 1104 | 414 |
| hMDK Ex5R (o) | GCTCTGGGACTCACATTGCT | | | | | | | | |
| hMDK Ex2F (o) | AGGCTTCCTCCTCCTCACC | 621 | 621 | 621 | 621 | 621 | 453 | 1066 | 376 |
| hMDK Ex5R (i) | TCCCAGAATCCCTTGTCCCT | | | | | | | | |
| hMDK Ex2F (i) | CGCGGTCGCCAAAAAGAAAG | 614 | 614 | 614 | 614 | 614 | 446 | 1059 | 369 |
| hMDK Ex5R (o) | GCTCTGGGACTCACATTGCT | | | | | | | | |
| hMDK Ex2F (i) | CGCGGTCGCCAAAAAGAAAG | 576 | 576 | 576 | 576 | 576 | 408 | 1021 | 331 |
| hMDK Ex54 (i) | TCCCAGAATCCCTTGTCCCT | | | | | | | | |

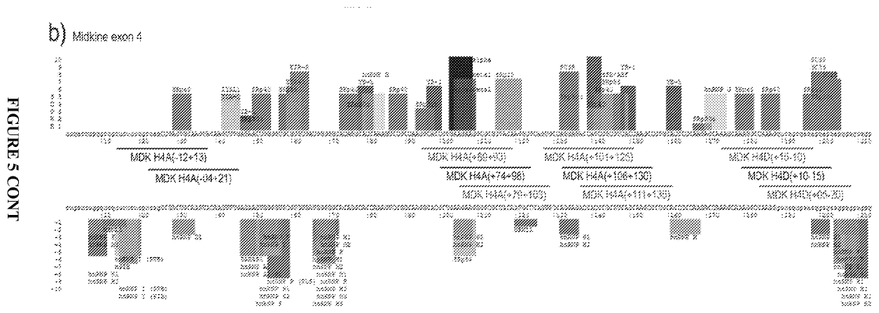

The six primers sets were optimised by altering PCR methods, annealing temperature, cycle number, and cell type (see FIG. 4). The optimised amplification conditions were identified as TaKaRa La Taq PCR system using 2×GC buffer I, and the following thermocycling conditions; 94° C. for 1 minute followed by 25-27 cycles of 94° C. for 30 seconds, 62° C. for 30 seconds and 72° C. for 2 minutes. The primer sets containing MDK Ex1F should amplify transcripts 203, 204 and 213, while all other primer sets should amplify all transcripts. When using the optimised TaKaRa PCR protocol, each primer set amplifies a single product roughly the size expected of the transcripts containing all four coding exons.

### Example 2: SSO Design and Synthesis

The first generation of MDK SSOs are shown in Table 6. The factors considered in the design strategy included optimal overlap with splice enhancer sequence motifs identified using the Splice Aid software, while aiming to minimise runs of greater than 3 Cs to avoid G blocks in the antisense SSOs, biased G+C compositions and secondary structures in the mRNA. After screening the generation 1 SSO sequences, additional SSOs were created by ‘microwalking’ around the effective sequences. Microwalking refers to moving the target sequence up- or down-stream of the original sequence. In this case, the second-generation SSOs were designed by moving 5 or 10 bases in each direction of the first-generation sequences that induced exon skipping (see Table 6). A 20-mer version of the most effective exon 3 SSO was also ordered. All SSOs were ordered as 2′-O-methyl modified bases on a phosphorothioate backbone (2′OMe-PS) from SynGenis (WA, Australia). The target sequence of these SSOs in relation to splicing factor binding site motifs predicted using SpliceAid 1 are shown in FIG. 5.

TABLE 6
List of 2′OMe-PS SSOs targeting exons 3 and 4 of MDK.

| | | |
|---|---|---|
| AO Name | Sequence (5′-3′) | GC% |

Generation 1

| | | |
|---|---|---|
| MDK H3A(−12+13) | UUCUUCACCUUAUCUACAACGCGCG | 48 |
| MDK H3A(−04+21) | GGCCGCCCUUCUUCACCUUAUCUAC | 56 |
| MDK H3A(+89+113) | GGCCCCGCAGGUGCCCUCGCGGAAA | 76 |
| MDK H3A(+143+167) | UCCAAACUCCUUCUUCCAGUUGCAG | 48 |
| MDK H3D(+15−10) | CCCGCCUCACCUCCAAACUCCUUCU | 60 |
| MDK H4A(−12+13) | UACUUGCAGUCGGCUGGCGAGAGAG | 60 |
| MDK H4A(−04+21) | CAAACUUGUACUUGCAGUCGGCUGG | 52 |
| MDK H4A(+74+98) | AGCAUUGUAGCGCGCCUUCUUCAGG | 56 |
| MDK H4A(+106+130) | GGCUUGGUGACGCGGAUGGUCUCCU | 64 |
| MDK H4D(+10−15) | UCUCCUUUCGCUGACCUUUGGCCUU | 52 |

Generation 2-Microwalked

| | | |
|---|---|---|
| MDK H3A(+133+157) | UUCUUCCAGUUGCAGGGCACCCUGC | 60 |
| MDK H3A(+138+162) | ACUCCUUCUUCCAGUUGCAGGGCAC | 56 |
| MDK H3D(+21−04) | UCACCUCCAAACUCCUUCUUCCAGU | 48 |
| MDK H3D(+10−15) | UGCGCCCCGCCUCACCUCCAAACUC | 68 |
| MDK H3D(+05−20) | CUGACUGCGCCCCGCCUCACCUCCA | 72 |
| MDK H4A(+69+93) | UGUAGCGCGCCUUCUUCAGGGUGCC | 64 |
| MDK H4A(+79+103) | CACUGAGCAUUGUAGCGCGCCUUCU | 56 |
| MDK H4A(+103+125) | GGUGACGCGGAUGGUCUCCUGGCAC | 68 |
| MDK H4A(+111+135) | UGCAGGGCUUGGUGACGCGGAUGGU | 64 |
| MDK H4D(+15−10) | UUUCGCUGACCUUUGGCCUUUGCUU | 48 |
| MDK H4D(+05−20) | CCCCUUCUCCUUUCGCUGACCUUUG | 56 |

### Example 3: 2′OMe-PS SSO Screening in Huh7

### Initial SSO Screening in Huh7

The human hepatoma-derived HuH-7 cell line (Huh7) was chosen for initial SSO screening due to their reported expression of MDK and ease of transfection. Huh7 cells were transfected with 2′OMe-PS SSOs using Lipofectamine 3000 at three concentrations: 200 nM, 100 nM and 50 nM. Cells were lysed and collected for RNA extraction and analysis after a 24-hour incubation period.

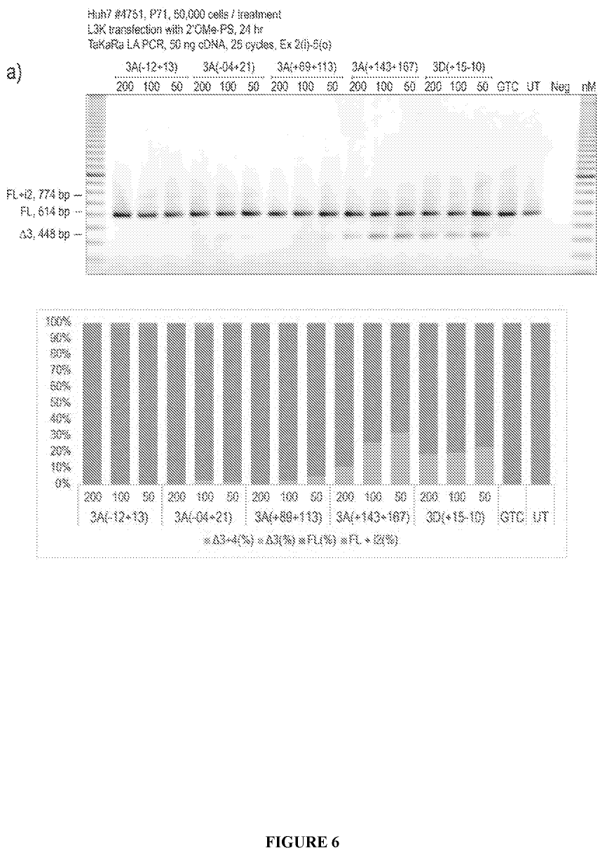

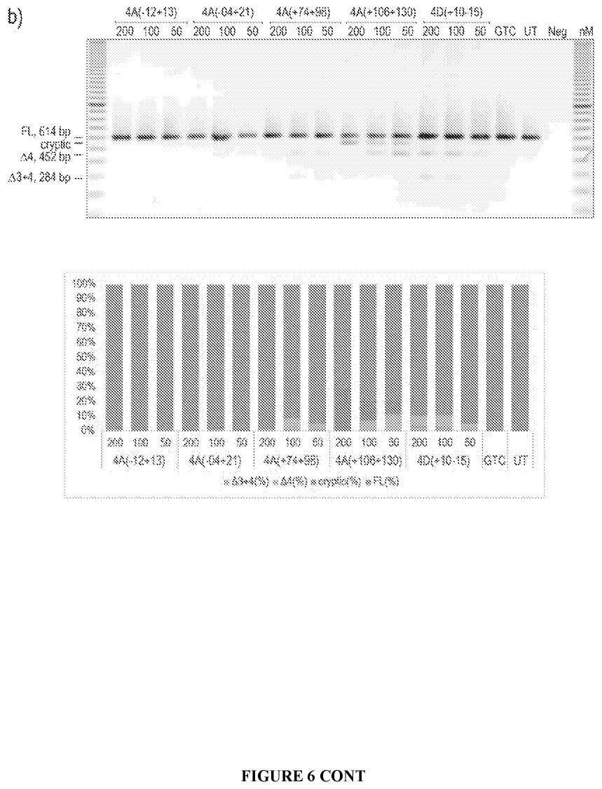

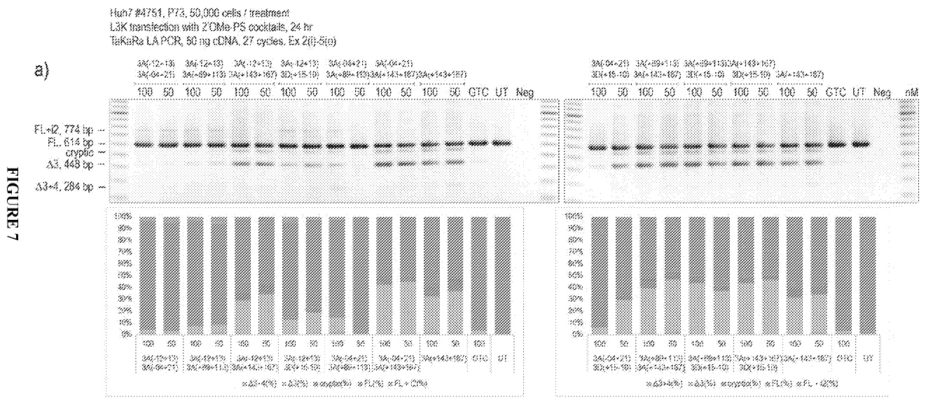

This initial SSO screen revealed that for both exon 3 and exon 4 the two SSOs closest to the donor splice site were the most effective at inducing exon skipping (see FIG. 6). Skipping of exon 3 (Δ3) was more efficient than exon 4 (Δ4) with approximately 32% Δ3 products after transfection with 3A(+143+167) at 50 nM (see FIG. 6a). The highest proportion of Δ4 products (11%) was achieved with 50 nM of 4A(+106+130) (see FIG. 6b). The exon 4 SSOs also induced skipping of both exon 3 and 4 (Δ3+4) as well as an intermediate band that is possibly cryptic splicing (see FIG. 6b). The first two SSOs targeting exon 3 also result in a small amount of PCR products with intron 2 inclusion (FL+i2).

### Cocktails in Huh7

Huh7 cells were transfected with two 2′OMe-PS SSOs simultaneously as a cocktail. It is often observed that two SSO sequences that are inefficient individually induce efficient skipping when used as a cocktail. Two concentrations, 100 nM and 50 nM, were assessed. Each SSO was used in equimolar amounts within these total cocktail concentrations, e.g., 50 nM of each SSO for a total cocktail concentration of 100 nM. Cells were lysed and collected for RNA extraction and analysis after 24 hours.

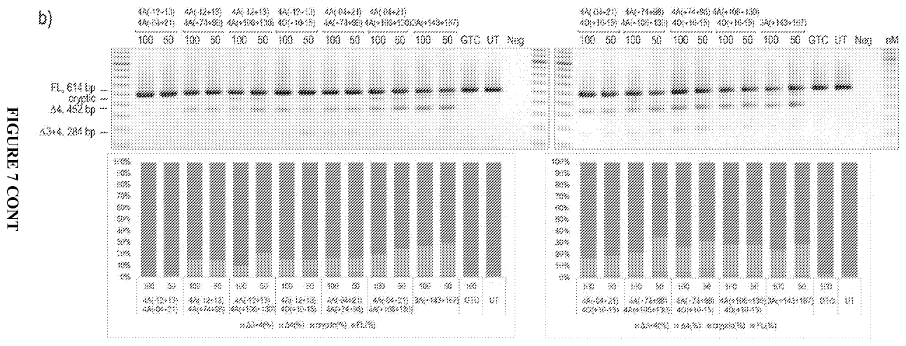

The SSO cocktails resulted in a similar splicing pattern to individual SSOs. The two SSOs closest to the donor splice site induce the most efficient skipping for exon 3 and exon 4. Despite the concentration of each SSO being lower than that previously tested, the 50 nM total concentration treatments were, for the most part, the most effective and induced a higher proportion of skipped products. Combining 3A(+143+167) with any other exon 3 SSO resulted in greater than 40% Δ3 products (see FIG. 7a). Most of the cocktails for exon 3 and exon 4 resulted in a small proportion of Δ3+4 products; however, these products were also observed in both the GTC and UT samples suggesting this may be naturally occurring skipping (see FIG. 7). The combination of 4A(+106+130 and 4D(+10−15) was most effective at inducing exon 4 skipping resulting in 26% Δ4 products (see FIG. 7b).

### Lower Concentrations in Huh7

Since previous screening experiments showed that the lower 50 nM concentration was effective and for exon 3 SSOs the most effective, a titration of lower concentrations was evaluated. Huh7 cells were transfected with 2′OMe-PS SSOs using Lipofectamine 3000 at three concentrations: 50 nM, 25 nM and 12.5 nM. Cells were lysed and collected for RNA extraction and analysis 24 hours after transfection.

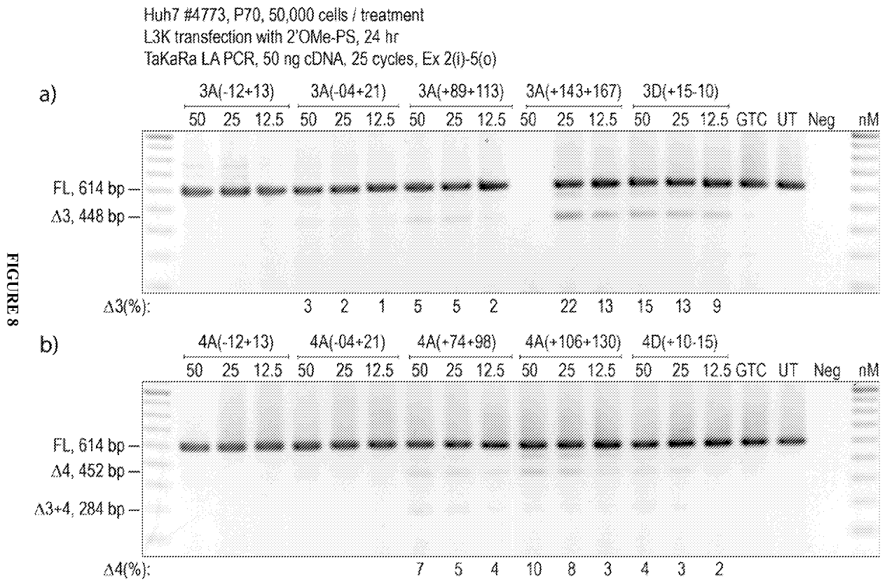

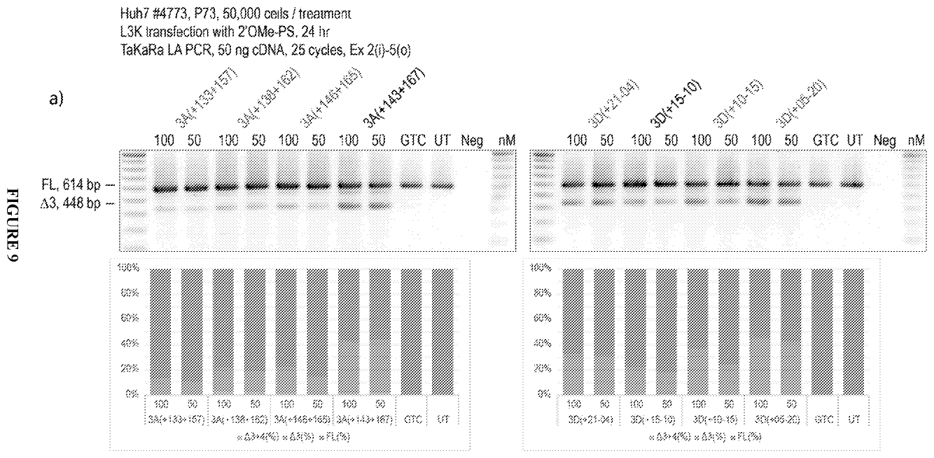

Both the 3A(+143+167) and 3D(+15-10) sequences induced exon skipping down to 12.5 nM in a dose-dependent manner, with the 50 nM concentration causing the most exon 3 skipping (see FIG. 8a). Skipping of exon 4 was also dose-dependent, with 50 nM inducing the highest proportion of the Δ4 products; however, this skipping was less effective than exon 3, with up to 10% Δ4 products (see FIG. 8b).

### Microwalked SSOs in Huh7

The second generation of MDK SSOs was designed by ‘microwalking’ around the effective generation 1 SSO sequences. Microwalking refers to moving the target sequence up- or down-stream of the original sequence. In this case, the second-generation SSOs were designed 5 or 10 bases in each direction of the first-generation sequences that induced exon skipping (Table 6). A 20-mer version of the most effective exon 3 SSO was also synthesised. All SSOs were ordered as 2′-O-methyl modified bases on a phosphorothioate backbone (2′OMe-PS) from SynGenis (WA, Australia). Huh7 cells were transfected with the microwalked and corresponding original 2′OMe-PS SSOs using Lipofectamine 3000 at two concentrations: 100 nM and 50 nM. Cells were lysed and collected for RNA extraction and analysis 24 hours after transfection.

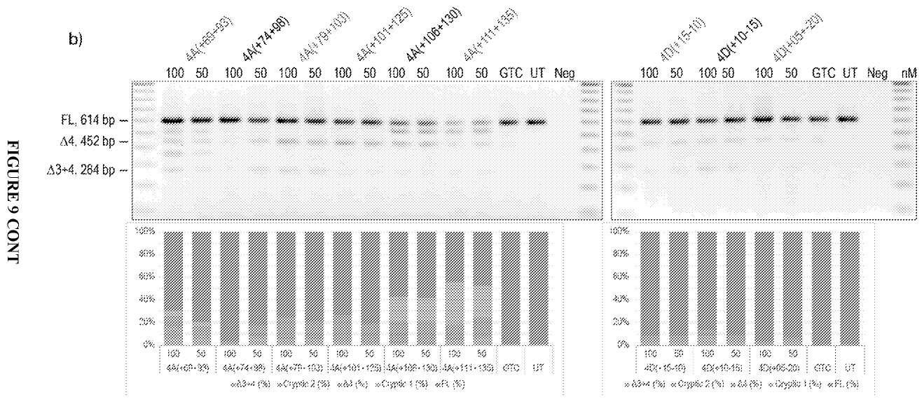

The exon 3 SSOs microwalked from 3A(+143+167) toward the acceptor site were less effective than the original sequence, as was the 20-mer sequence (see FIG. 9a). In contrast, the new 3D(+05-20) sequence effectively induced up to 45% exon 3 skipping (see FIG. 9a). The SSOs microwalked around the original 4A(106+130) demonstrate an interesting phenomenon. On one side, 4A(+111+145) induces more cryptic splicing and less exon 4 skipping than the original, and on the other 4A+101+125) induces more exon 4 skipping without causing cryptic splicing (see FIG. 9b). A similar result is seen with the sequences microwalked around 4A(+74+98), where 4A(+69+93) induces multiple non-specific skipped products and 4A(+79+103) induces more specific exon 4 skipping (see FIG. 9b).

### Exon 3+4 Cocktails in Huh7

Huh7 cells were transfected with two 2′OMe-PS SSOs simultaneously as a cocktail; however, each cocktail was made up of one SSO for each exon 3 and exon 4. The aim was to reduce the proportion of full-length products as much as possible. Two concentrations, 100 nM and 50 nM, were assessed. Each SSO was used in equimolar amounts within these total cocktail concentrations, e.g., 50 nM of each SSO for a total cocktail concentration of 100 nM. Cells were lysed and collected for RNA extraction and analysis after 24 hours.

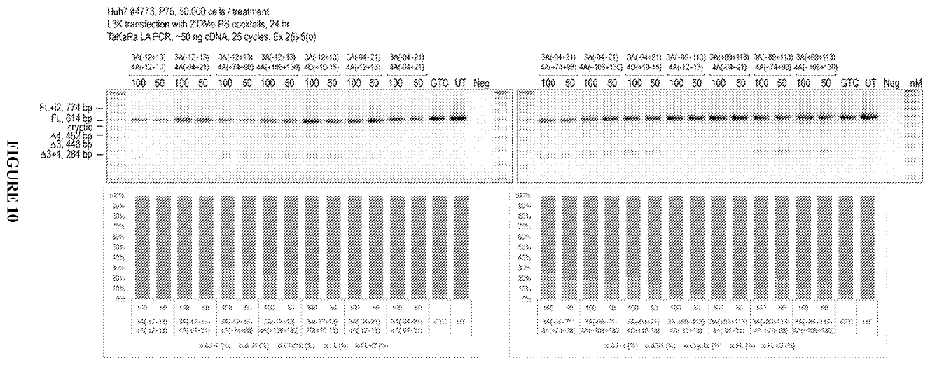

Treatment with exon 3+4 cocktails resulted in an expected splicing pattern with those cocktails that contain an effective SSO for either exon 3 or exon 4, inducing Δ3+4 skipping (see FIG. 10). The exception to this is the combination of 3A(+143+167) or 3D(+15-10) with either of the two SSOs targeting the exon 4 acceptor site; these cocktails induce only single exon skipping, likely exon 3 (see FIG. 10). None of the cocktails was as effective as the 3A(+143+167) SSO used as a positive control, which reduced the proportion of FL products to 58%. The combination of 3A(+143+167) and 4D(+10-15) was the most effective cocktail reducing the proportion of FL products to ˜60% at 100 nM (see FIG. 10).

### Example 4: 2′OMe-PS SSO Screening in SHSY5Y

The 2′OMe-PS SSO screening performed in Huh7 cells was repeated in the neuroblastoma-derived SHSY5Y cell line. SHSY5Y cells were transfected with either individual SSOs at 200, 100 and 50 nM or two-SSO cocktails at 100 and 50 nM. Cells were lysed and collected for RNA extraction and analysis after 24 hours.

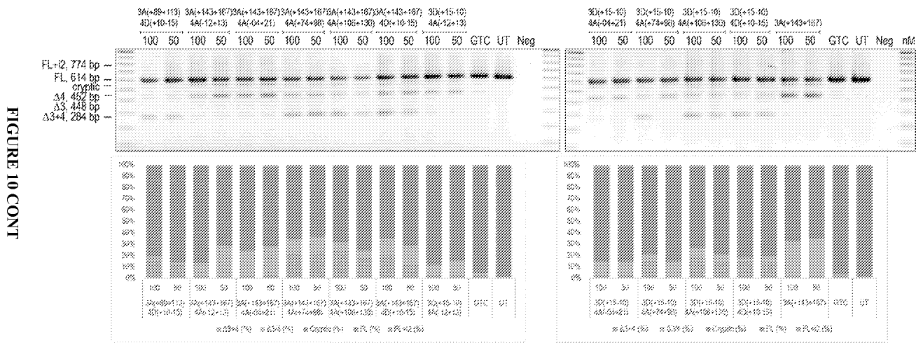

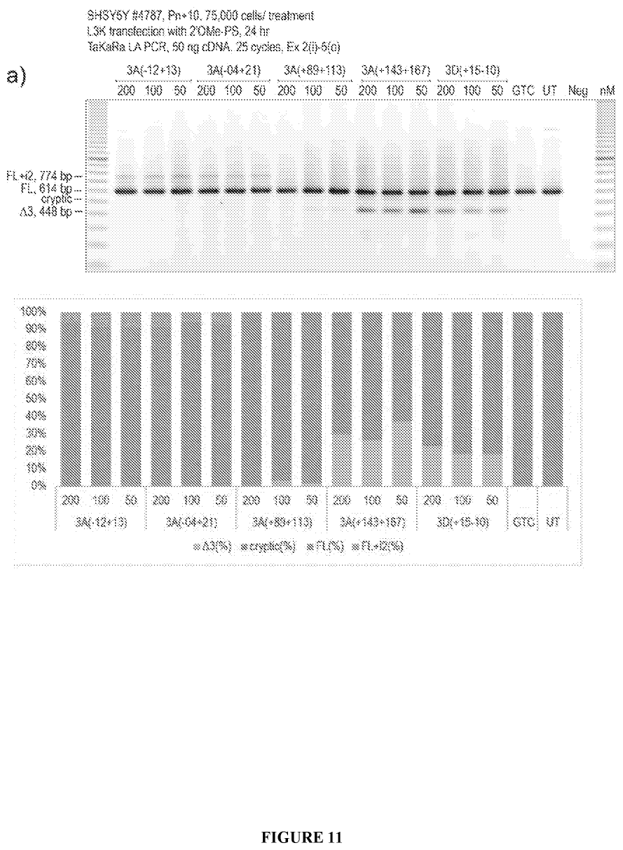

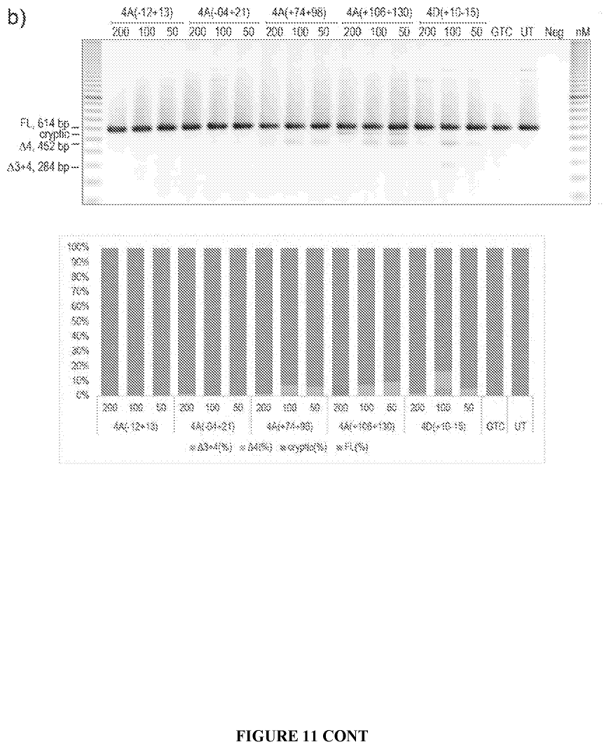

After transfection of SHSY5Y cells with individual SSOs, the splicing pattern was similar to that observed in Huh7 cells. The 3A(+143+167) and 3D(+15-10) SSOs induced the highest proportion of Δ3 products, with up to 37% and 23%, respectively. Interestingly the strong dose-response observed in Huh7 cells was not seen in SHSY5Y cells with the 200 nM and 50 nM concentrations resulting in a similar proportion of exon skipped products (see FIG. 11a). The FL+i2 product was also more prominent in SHSY5Y cells making up more than 5% of PCR products in all concentrations after transfection with either of the SSOs covering the acceptor splice site (see FIG. 11a). Exon 4 skipping with individual SSOs was similarly inefficient in both cell lines, with both; very little of the desired Δ4 product as well as production of the Δ3+4 and cryptic products (see FIG. 11b).

While the pattern of splicing products after transfection with two-SSO cocktails is similar between Huh7 and SHSY5Y cells, the proportion of skipped products is much lower in SHSY5Y cells (see FIG. 11c). We also note that the higher 200 nM concentration is more effective in SHSY5Y, while the lower 50 nM concentration is most effective in Huh7 cells. This reduction in efficiency could be due to the increased number of cells used per transfection from 50,000 for Huh7 to 75,000 for SHSY5Y. However, it is also possible that this is the result of lower transfection efficiency in SHSY5Y cells compared to Huh7 cells.

### Example 5: 2′OMe-PS SSO Screening in HMC-1

### Initial Transfection of Promising AOs in HMC-I

The human mast cell line (HMC-1) was transfected with the most promising SSOs for each MDK exon using Lipofectamine 3000. The transfection protocol was modified since these cells had not previously been transfected and are suspension cells. HMC-1 cells were transferred the day before transfection into 24-well plates at a 6×10⁵ cells/ml density. On the day of transfection, the delivery complex containing the SSO and Lipofectamine 3000 diluted in 50 μl of OptiMEM was incubated for 15 minutes, as recommended by the manufacturer, and then added directly to cells in their growth medium. This protocol differs from that used for adherent cell lines in which the cells are similarly seeded 24 hours before transfection. However, after the 15-minute incubation, the delivery complex is further diluted in OptiMEM that is added to the cells replacing their growth media. The HMC-1 cells were lysed and collected for RNA extraction and analysis 24 hours after transfection.

Both the single SSO and the two-SSO cocktail targeting exon 3 effectively induced exon skipping (see FIG. 12a). In contrast, no exon 4 skipping was observed (see FIG. 12a). The SMN positive control revealed that the transfection efficiency was poor with only ˜50% SMN exon 7 skipping (see FIG. 12b). An efficient transfection would expect to see 80-100% SMN skipping. In addition, the natural exon 7 skipping seen in the untreated sample is often seen in-house when cells are stressed.

Due to contaminating gDNA, the RNA samples were treated with ezDNAse before cDNA synthesis and PCR amplification. Despite the ezDNAse treatment efficacy (FIG. 12.c), the more oversized PCR products correlating roughly in size to the genomic DNA are still present, suggesting unprocessed transcripts are held in the HMC-1 Mast cells.

Second Transfection with Fewer Cells

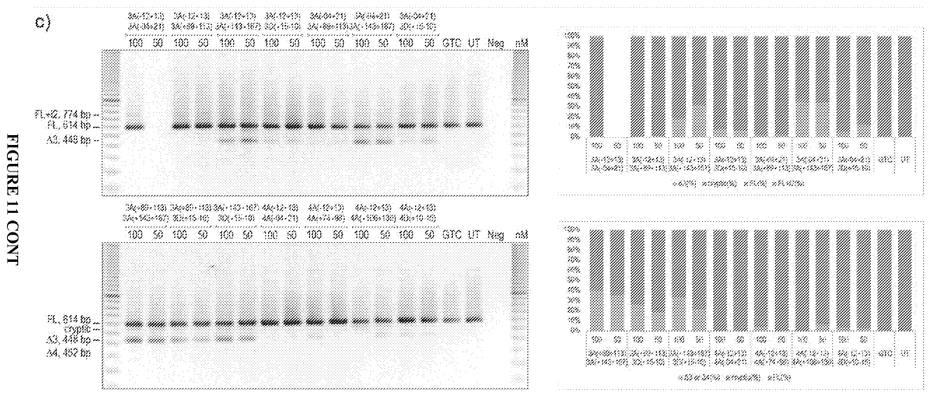

The transfection of HMC-1 cells was repeated using 1×10⁶ cells per treatment rather than 6×10⁶ in an attempt to increase the transfection efficiency. Despite the increase in efficiency indicated by the 79% SMN exon 7 skipping (see FIG. 13b), only a slight rise in skipping efficiency was observed, and the exon 4 SSOs remained ineffective (see FIG. 13a).

### Microwalked Exon 4 SSOs in HMC-1 Cells

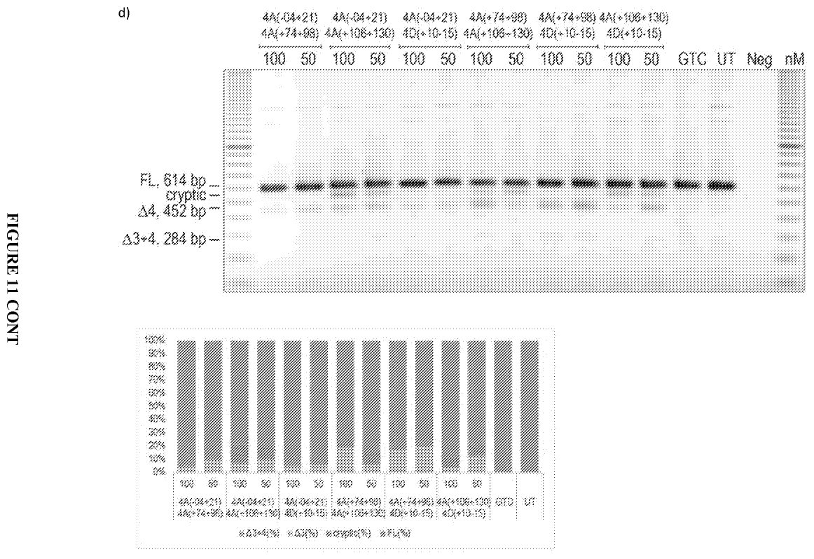

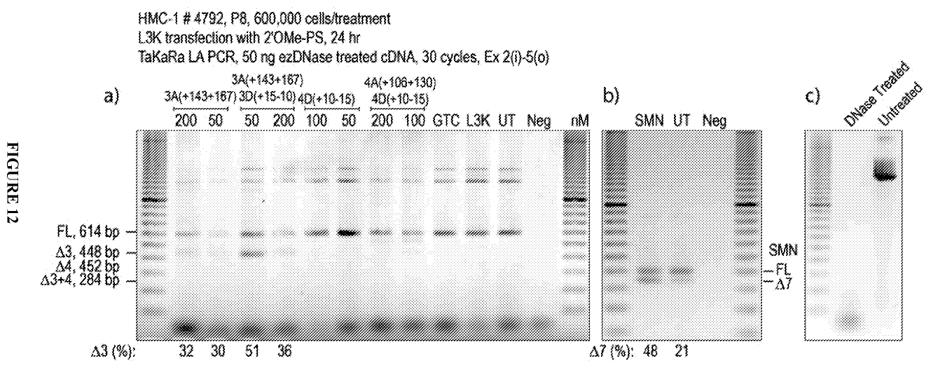

The two microwalked SSOs for exon 4 that were identified as more effective than the original sequences (see FIG. 9b) were transfected into the HMC-1 cells. Despite the poor transfection efficiency indicated by the 3A(+143+167) positive control SSO, the microwalked exon 4 SSOs did successfully induce exon 4 skipping (see FIG. 14). A more suspension-cell-line friendly transfection method such as electroporation may be required to increase the transfection efficiency of HMC-1 cells.

### Example 6: PMO Evaluation

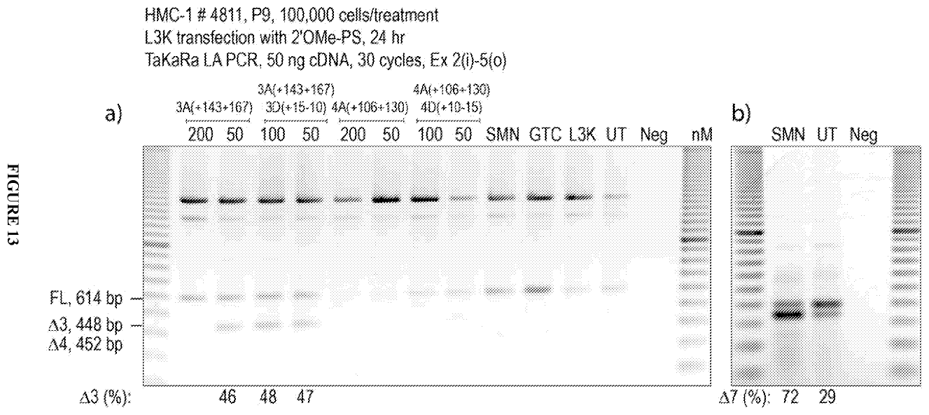

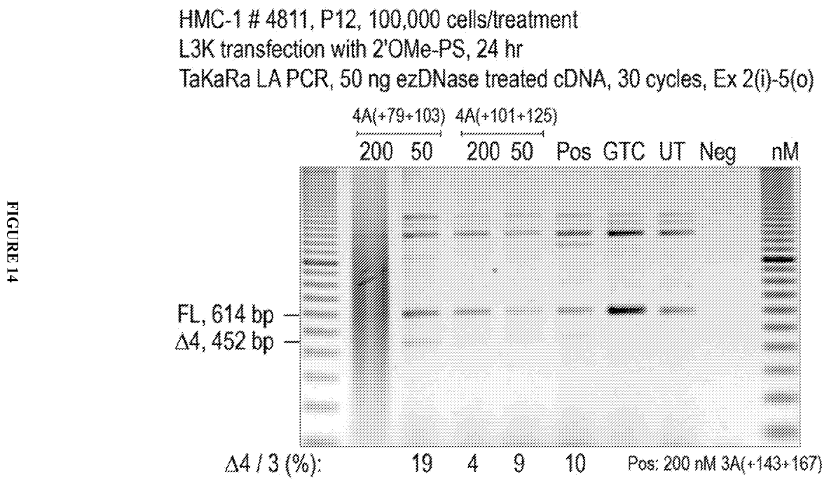

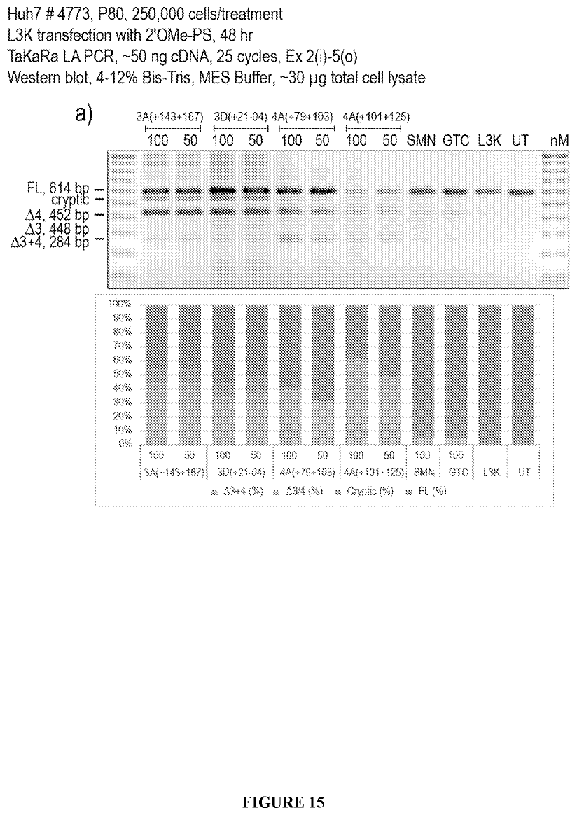

The most promising SSO sequence(s) for each exon were ordered as the phosphorodiamidate morpholino oligomer (PMO) chemistry (Table 6). Selection of SSO sequence(s) for PMO synthesis from each exon was based on the results of the previous experiments carried out in HuH7, SHSY5Y and HMC-1 cells outlined above and confirmed in final testing in HuH7 cells (FIG. 15). This identified 3A(+143+167), 4A(+79+103) and 4A(+101+125) as the best candidates for PMO evaluation, while 3D(+21-04) was not developed as a PMO SSO due to unfavourable features of the RNA sequence around this region of Exon 3.

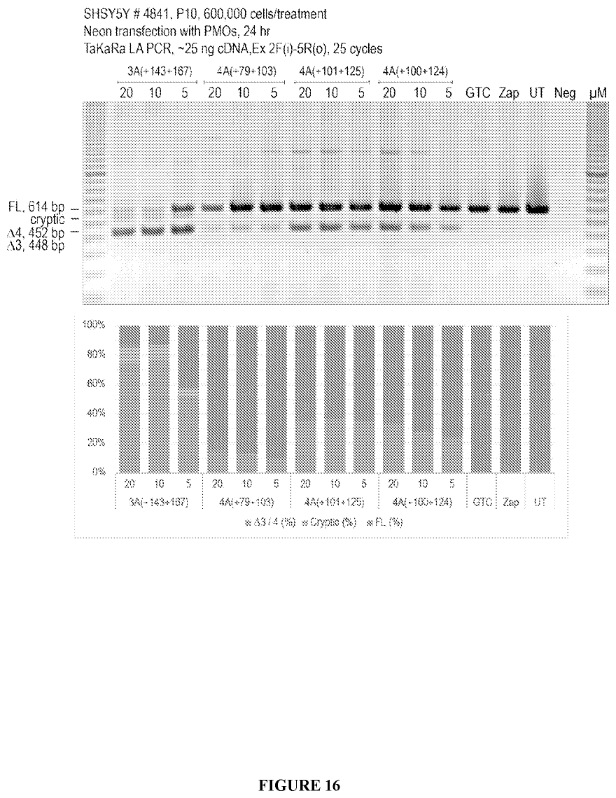

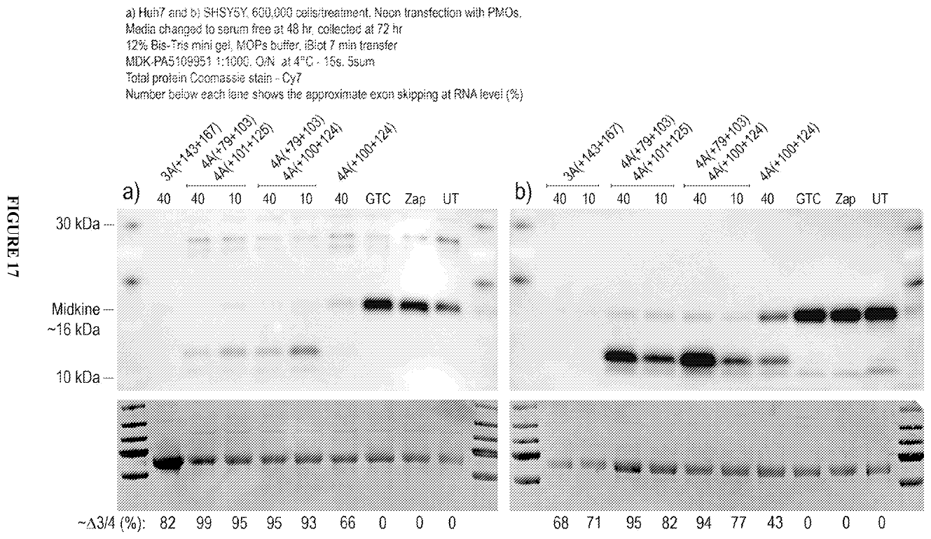

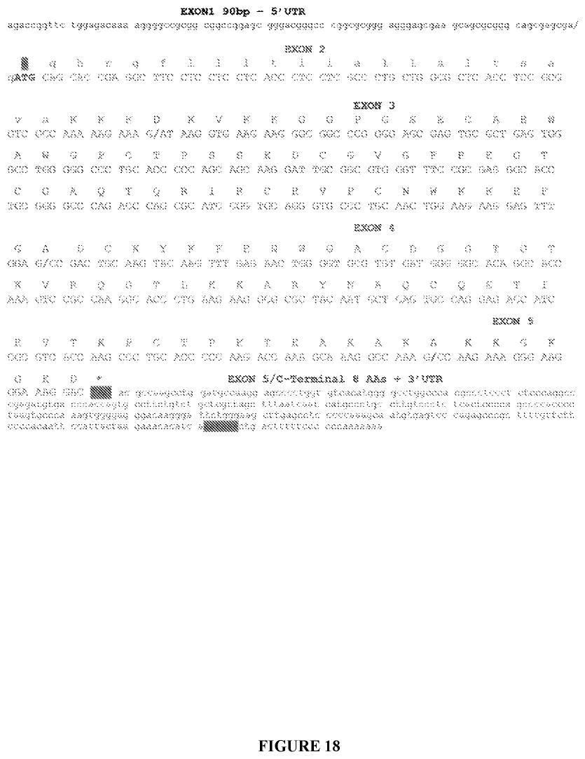

One additional sequence MDK 4A(+100+124), was also designed due to concerns from Gene Tools over the proportion of G nucleotides in the MDK 4A(+101+125) sequence (40%) that was above their recommended upper limit (36%). Please note that the MDK H4A(+79+103) sequence was also flagged for high self-complementarity, which can lead to dimer formation and lower antisense activity. SHSY5Y cells were transfected with the PMOs using the Neon electroporation transfection method. Three concentrations were evaluated; 20 μM, 10 μM and 5 μM, these concentrations are calculated in the 10 μl Neon tip and roughly correlate to 200 nM, 100 nM and 50 nM in 1 ml. The cells were collected 24 hours after transfection due to concern over the proportion of cell death (˜50%) that could lead to cell proliferation and dilution of the PMOs effects. The PMO SSO directed to exon 3 reached around 80% skipping efficiency, while the PMO SSOs directed to exon 4 reach around 40% skipping efficiency (see FIG. 16).

TABLE 6
List of PMO SSOs targeting exons 3 and 4 of MDK

| Oligo Label | AO Sequence (5′-3′) |
|---|---|
| MDK H3A(+143+167) | TCCAAACTCCTTCTTCCAGTTGCAG |
| MDK h4A(+79+103) | CACTGAGCATTGTAGCGCGCCTTCT |
| MDK H4A(+101+125) | GGTGACGCGGATGGTCTCCTGGCAC |
| MDK H4A(+100+124) | GTGACGCGGATGGTCTCCTGGCACT |

### Example 7: Western Blot Evaluation

Huh7 and SHSY5Y cells were transfected with the most promising exon 3 and 4 AOs as both individuals and two-AO cocktails using lipofectamine 3000. The cells were incubated for 48 hours after transfection and then collected for RNA or protein analysis. RNA analysis revealed that the transfection was efficient with induction of both exon 3 and exon 4 skipping (FIG. 17, top panel). Western blotting clearly shows the reduction in full length midkine in the control lanes (GTC, ZAP, UTC) relative to the short form in all tracks with Exon 4 SSOs (FIG. 17, bottom panel).

FIG. 17 provides evidence of the production of a truncated human midkine protein with an exon 4 deletion.

### Example 8: Midkine mRNAs

Midkine mRNAs comprising the full length midkine coding region (SEQ ID NO: 96) including the natural start codon and peptide leader sequence linked to a 5′UTR; CAP structure; 3′UTR and poly AAA sequence were synthesized by in vitro transcription using a standard plasmid template and RNA polymerase.

Deletion mRNAs encoding truncated midkine proteins lacking 14, 25, 34, 41 and 58 amino acids from the C-terminus were designed (set forth in SEQ ID NOs: 86, 88, 90, 92 and 94 respectively). The deletion mRNAs included the same 5′UTR, CAP structure, 3′UTR and poly AAA sequences as those used for the full length midkine sequence.

Deletion mRNA encoding truncated midkine proteins lacking 41 amino acids from the C-terminus (set forth in SEQ ID NO: 92) was included in the experiments as a comparator as it has been reported in the literature (Dianat et al. 2014). Whilst Dianat et al. 2014 refers to a variant lacking its last 40 amino acids, the skilled person would understand from the nomenclature MDKΔ81-121 referred to in the paper, that in fact the midkine is lacking its last 41 amino acids.

### Example 9: Detection of Truncated Midkine Protein Produced by Midkine mRNA Deletions in Cancer Cells

Transfection of Breast Cancer Cells with Midkine mRNAs:

2,000 MDA-MB-231 breast cancer cells per well were plated in 1 mL DMEM and 10% FBS in a 96-well plate. Cells were incubated overnight at 37° C. in 5% CO₂.

The transfection media was prepared by combining 1 μL of Lipofectamine MessengerMax (Cat. #LMRNA001), 0.2 ng/1000 cells ng midkine deletion mRNA template (each well received one of the deletion mRNA sequences set forth in SEQ ID NOs: 86, 88, 90, 92, 94 and 96), and Opti-MEM up to final volume of 50 μL.

The cells were incubated for 5-6 hours in the transfection media at 37° C. in 5% CO₂ and then 150 μL of DMEM and 10% FBS was added onto each well containing 50 μL Opti-MEM and incubation continued for a further 72 hrs. Cell viability was assessed by adding Alamar Blue to the plate and absorbance captured at 570-595 nm after 13 hours.

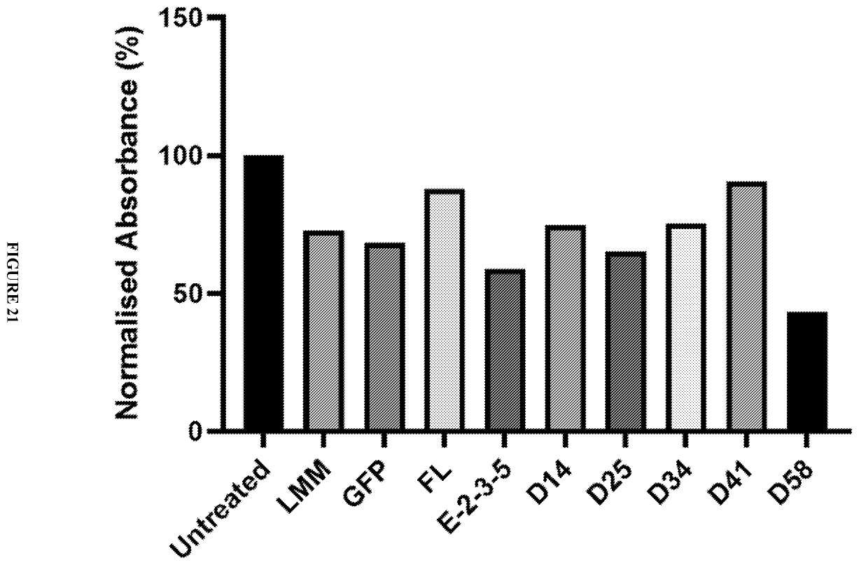

Midkine mRNA deletions Δ14, Δ25, Δ34 and Δ58 all showed a reduction in cancer cell viability compared to the full length midkine mRNA indicating that truncated midkine lacking 14, 25, 34 and 58 amino acids from the C-terminus interferes with breast cancer cell proliferation (see FIG. 21). Furthermore, we note that midkine mRNA deletion Δ41 was comparable to full length midkine and as such not a suitable candidate.

### Western Blotting

18 μL of the conditioned media was loaded together with a protein loading buffer onto precast 4-15% gradient Bio-Rad Gel. The proteins were separated and then immobilised on a membrane using Bio-Rad transfer system.

Membranes were incubated with MDK antibody overnight (Invitrogen PA5-109951) at 1:1000 followed by secondary rabbit antibody for 1 hour at 1:5000 in 5% Skim Milk blocking buffer. Antibodies were diluted in 2.5% Skim Milk. Detection of the midkine specific bands was carried out using chemiluminescence.

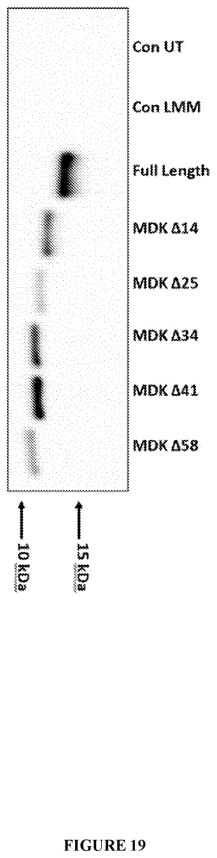

A progressive reduction in size of the bands from full length midkine to Δ14 and ultimately Δ58 indicates the expected size when between 14 and 58 amino acids are removed from the C-terminus of truncated midkine protein (see FIG. 19).

### Example 10: Migration of Breast Cancer Cells is Reduced by Midkine mRNAs with 14 or 58 Amino Acid Deletion from the C-Terminus

### Cell Migration Assay

50 000 MDA-MB-231 breast cancer cells per well were plated in 100 μL DMEM and 10% FBS in a 96-well plate. The cells were incubated overnight at 37° C. in 5% CO₂ in incubator.

The transfection media was prepared by combining Lipofectamine MessengerMax and mRNA encoding the Δ14 or Δ58 deletion variant at a concentration of approximately 1 ng mRNA per 1000 cells. Cells were transfected in 50 μL of Opti-MEM

The cells were incubated for 5-6 hours at 37° C. in the presence of 5% CO₂ whereupon 150 μL of DMEM+10% FBS was added onto each well containing 50 μL Opti-MEM. Media was removed at 24 hours post-transfection and then 100 μL PBS was added. The semiconfluent monolayer of cells was then scratched using WoundMaker System, followed by one wash of PBS. 150 μL of the transfection media was returned to each well. Then the plate was placed into an IncuCyte and imaged every hour for 12 hours to determine the migration of cells into the gap. The area under the curve was calculated by quantifying the wound width at hourly intervals (Table 7).

Midkine mRNA deletion variants were compared to the control green fluorescent protein (GFP) mRNA or Full length midkine mRNAs. Midkine mRNAs Δ58 and Δ14 (SEQ ID NOs: 86 and 92 respectively) showed significant interference with breast cancer cell migration. However, interestingly, midkine mRNA Δ41 did not affect breast cancer cell migration at all.

TABLE 7
Total Area and Standard error of the mean
(SEM) results for Cell Migration Assay

| | | Full | | | | | |
|---|---|---|---|---|---|---|---|
| | | Length | Δ14 | Δ25 | Δ34 | Δ41 | Δ58 |
| | GFP | MDK | MDK | MDK | MDK | MDK | MDK |
| | mRNA | mRNA | mRNA | mRNA | mRNA | mRNA | mRNA |
| Total | 1003 | 1123 | 1706 | 1036 | 768 | 924 | 1992 |
| Area | | | | | | | |
| SEM | 334 | 367 | 300 | 309 | 263 | 292 | 330 |

### Example 11: Proliferation of Liver Cancer Cells is Reduced by Midkine Deletion mRNA

10 000 Huh-7D12 cells per well were plated in 100 μL DMEM and 10% FBS in a 96-well plate. The cells were incubated overnight at 37° C. in the presence of 5% CO₂.

Transfection media was prepared based on initial cell densities used in Example 9. Lipofectamine MessengerMax was added, together with an mRNA encoding one of the midkine deletion variants at a concentration of approximately 1 ng mRNA per 1000 cells. Cells were transfected in 50 μL of Opti-MEM. The cells were incubated for 5-6 hours at 37° C. in the presence of 5% CO₂. 150 μL of DMEM and 10% FBS was added onto each well containing 50 μL Opti-MEM. 24 hours post-transfection, the media was removed and PBS was used to wash the cells once. Next, 150 μL of the transfection media was returned to each well and the plate was placed into incubator at 37° C. in 5% CO₂ for 72 hours. Then Alamar Blue was added to the plate and absorbance was captured at 570-595 nm after 5 hours to determine number of viable cells.

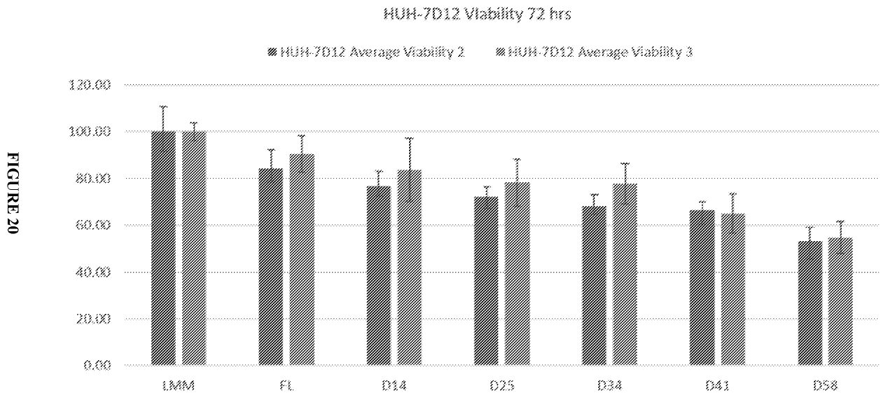

Midkine mRNA deletion Δ58 reduced cell viability compared to the full length midkine mRNA indicating that truncated midkine lacking 58 amino acids from the C-terminus interferes with liver cancer cell proliferation (see FIG. 20).

It will be appreciated by persons skilled in the art that numerous variations and/or modifications may be made to the above-described examples, without departing from the broad general scope of the present disclosure. The present examples are, therefore, to be considered in all respects as illustrative and not restrictive.

1. A composition comprising:
(i) a truncated human midkine protein in which the amino acid sequence encoded by exon 4 of human midkine is absent, partially absent or substantially absent; and/or
(ii) a polynucleotide encoding the truncated human midkine protein at (i).

2. The composition of claim 1, wherein about 14, about 25, about 34 or about 58 contiguous amino acids of the C-terminus of human midkine protein are absent from the truncated human midkine protein.

3. The composition of claims 1 or 2, wherein human midkine protein comprises the amino acid sequence set forth in SEQ ID NO: 75.

4. The composition of any one of claims 1 to 3, wherein about 14 contiguous amino acids of the C-terminus of human midkine protein are absent from the truncated human midkine protein.

5. The composition of claim 4, wherein the truncated human midkine protein comprises or consists of the sequence set forth in SEQ ID NO: 87.

6. The composition of any one of claims 1 to 3, wherein about 58 contiguous amino acids of the C-terminus of human midkine protein are absent from the truncated human midkine protein.

7. The composition of claim 4, wherein the truncated human midkine protein comprises or consists of the sequence set forth in SEQ ID NO: 95.

8. The composition of any one of claims 1 to 7, wherein the polynucleotide is an mRNA.

9. The composition of claim 8 when appended to any one of claims 1 to 5, wherein the polynucleotide is an mRNA encoding a truncated human midkine protein in which about 14 contiguous amino acids of the C-terminus of human midkine protein are absent, wherein the mRNA comprises or consists of a polynucleotide sequence having at least 80% identity to the sequence set forth in SEQ ID NO: 86.

10. The composition of claim 9, wherein the polynucleotide comprises the sequence set forth in SEQ ID NO: 86.

11. The composition of claim 9 or 10, wherein the polynucleotide sequence encodes an amino acid sequence set forth in SEQ ID NO: 87.

12. The composition of claim 8 when appended to any one of claims 1 to 3 and 6 or 7, wherein the polynucleotide is an mRNA encoding a truncated human midkine protein in which about 58 contiguous amino acids of the C-terminus of human midkine protein are absent, wherein the mRNA comprises or consists of a polynucleotide sequence having at least 80% identity to the sequence set forth in SEQ ID NO: 94.

13. The composition of claim 12, wherein the polynucleotide comprises the sequence set forth in SEQ ID NO: 94.

14. The composition of claim 12 or 13, wherein the polynucleotide sequence encodes an amino acid sequence set forth in SEQ ID NO: 95.

15. The composition of any one of claims 1 to 14, wherein the polynucleotide is codon optimised.

16. The composition of any one of claims 1 to 7, wherein the polynucleotide is a DNA sequence encoding the truncated midkine protein and the polynucleotide is operably-linked to a promoter and/or comprised within an expression vector.

17. The composition of any one of claims 1 to 15, wherein the polynucleotide is a mRNA which is formulated with a lipid nanoparticle (LNP).

18. The composition of any one of claims 1 to 17, further comprising a pharmaceutically acceptable carrier or diluent.

19. A method for producing a truncated human midkine protein, comprising culturing a cell comprising a polynucleotide as defined in claim 16 for a time and under conditions sufficient for the cell to produce the truncated human midkine protein, optionally comprising recovering the truncated human midkine protein from the culture.

19. A method for producing a truncated human midkine protein, comprising transfecting a polynucleotide as defined in any one of claim 1 to 15 into a cell under conditions sufficient for the cell to produce the truncated human midkine protein, optionally comprising recovering the truncated human midkine protein from the culture.

20. A method for inhibiting an interaction between human midkine and a ligand thereof on the surface of, or in a cell, said method comprising exposing the cell to the composition according to any one of claims 1 to 18.

21. The method of claim 20, wherein inhibition of the interaction between the human midkine and the ligand thereof reduces cancer cell viability and/or cell migration.

22. A method for inhibiting human midkine activity in a cell, said method comprising exposing the cell to the composition according to any one of claims 1 to 18.

23. A method for treating or preventing a midkine-related disease or disorder in a subject in need thereof, said method comprising administering to the subject the composition according to any one of claims 1 to 18.

24. The method of claim 23, wherein the midkine-related disease or disorder is an autoimmune disease, cancer, an inflammatory disease or multiple sclerosis.

25. The method of claim 24, wherein the midkine-related disease or disorder is cancer.

26. Use of the composition according to any one of claims 1 to 18 in the preparation of a medicament for treatment or prevention of a midkine-related disease or disorder selected from an autoimmune disease, cancer, or an inflammatory disease in a subject in need thereof.

27. The use of claim 26, wherein the medicament is for treatment of cancer.

28. Use of the composition according to any one of claims 1 to 18 to treat or prevent a midkine-related disease or disorder selected from an autoimmune disease, cancer, or an inflammatory disease in a subject in need thereof.

29. The use of claim 28, wherein the midkine-related disease or disorder is cancer.

30. The method of claim 25 or the use of claim 27 or 29, wherein the cancer is selected from the group consisting of esophageal cancer, thyroid cancer, urinary bladder cancer, colorectal cancer, cutaneous and uveal melanoma, squamous cell carcinoma, osteosarcoma B-cell malignancies, leukemia, head and neck cancer, gall bladder cancer, stomach cancer, pancreatic cancer, thoracic cancer, liver cancer, lung cancer, breast cancer, neuroblastoma, glioblastoma, uterine cancer, ovarian cancer, prostatic cancer, and Wilms tumor.

31. The method of claim 25 or the use of claim 27 or 29, wherein the cancer is breast cancer.

32. The method of claim 25 or the use of claim 27 or 29, wherein the cancer is liver cancer.

33. The method of any one of claims 23 to 24 and 30 to 32 use of any one of claims 26 to 32, wherein the subject is being treated with or will be treated with a chemotherapeutic agent.
